(19) United States
(12) Patent Application Publication
(10) Pub. No.: US 2011/0201608 A1
(43) Pub. Date: Aug. 18, 2011

(54) SUBSTITUTED NAPHTHYRIDINES AND USE THEREOF AS MEDICINES

(75) Inventors: Matthias Hoffmann, Mittelbiberach (DE); Thierry Bouyssou, Warthausen (DE); Georg Dahamnn, Attenweiler (DE); Harald Engelhardt, Ebreichsdorf (AT); Dennis Fiegen, Biberach (DE); Sandra Handschum, Biberach (DE); Silke Hobbie, Biberach (DE); Takeshi Kono, Osaka (JP); Ulrich Reiser, Wein (AT); Yayoi Sato, Hyogo (JP); Andreas Schnapp, Biberach (DE); Annette Schuler-Metz, Ulm (DE)

(73) Assignee: BOEHRINGER INGELHEIM INTERNATIONAL GMBH, Ingelheim am Rhein (DE)

(21) Appl. No.: 13/057,288

(22) PCT Filed: Jul. 23, 2009
(86) PCT No.: PCT/EP2009/059500

§ 371 Date: Apr. 6, 2011

(30) Foreign Application Priority Data
Aug. 5, 2008 (EP) 08161832.4

(51) Int. Cl. A61K 31/5377; C07D 471/04; A61K 31/496; A61P 11/06; A61P 19/02; A61P 1/04; A61P 17/06; A61P 35/00; A61P 17/00; A61P 11/00

(52) U.S. Cl. 514/234.5; 544/127; 544/362; 514/253.04

(57) ABSTRACT

The invention relates to new substituted naphthyridines of formula 1, as well as pharmacologically acceptable salts, diastereomers, enantiomers, racemates, hydrates or solvates thereof,

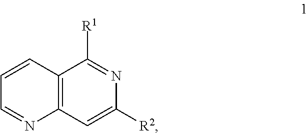

wherein
R¹ denotes a group A selected from among —O—R³, —NR³R⁴, —CR³R⁴R⁵, -(ethyne)-R³, —S—R³, —SO—R³ and SO₂—R³
or
R¹ denotes a group B selected from among
C₆₋₁₀-aryl,
five- to ten-membered, mono- or bicyclic heteroaryl with 1-3 heteroatoms selected independently of one another from among N, O and S; while this heteroaryl is linked to the structure according to formula 1 via either a C atom or an N atom,
three- to ten-membered, mono- or bicyclic, saturated or partially saturated heterocyclic group with 1-3 heteroatoms selected independently of one another from among N, O and S, while this heterocyclic group is linked to the structure according to formula 1 via either a C atom or an N atom,
   and
5- to 11-membered spiro group which may optionally contain 1, 2 or 3 heteroatoms selected independently of one another from among N, O and S, while this spiro group is linked to the structure according to formula 1 via either a C atom or an N atom,
   while this group B may optionally be substituted as described in claim 1
   and wherein R² is

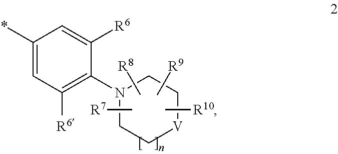

and R³, R⁴, R⁵, R⁶, R^{6′}, R⁷, R⁸, R⁹, R¹⁰, V, n and m may have the meanings given in claim 1, as well as pharmaceutical compositions containing these compounds.

The invention relates to new substituted naphthyridines of formula 1, as well as pharmacologically acceptable salts, diastereomers, enantiomers, racemates, hydrates or solvates thereof,

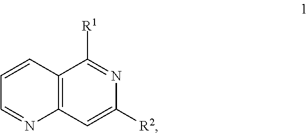

wherein
R¹ denotes a group A selected from among —O—R³, —NR³R⁴, —CR³R⁴R⁵, -(ethyne)-R³, —S—R³, —SO—R³ and SO₂—R³
or
R¹ denotes a group B selected from among
C₆₋₁₀-aryl,
five- to ten-membered, mono- or bicyclic heteroaryl with 1-3 heteroatoms selected independently of one another from among N, O and S; while this heteroaryl is linked to the structure according to formula 1 via either a C atom or an N atom,
three- to ten-membered, mono- or bicyclic, saturated or partially saturated heterocyclic group with 1-3 heteroatoms selected independently of one another from among N, O and S, while this heterocyclic group is linked to the structure according to formula 1 via either a C atom or an N atom,
   and
5- to 11-membered spiro group which may contain optionally 1, 2 or 3 heteroatoms selected independently of one another from among N, O and S, while this spiro group is linked to the structure according to formula 1 via either a C atom or an N atom,
   while this group B may optionally be substituted as described in claim 1
   wherein R² is

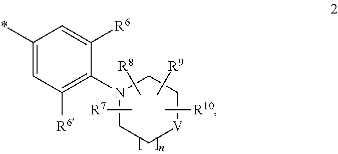

and R³, R⁴, R⁵, R⁶, R^{6′}, R⁷, R⁸, R⁹, R¹⁰, V, n and m may have the meanings given in claim 1, as well as pharmaceutical compositions containing these compounds.

### 1. BACKGROUND TO THE INVENTION

### 1.1 SYK-Inhibitors

The present invention describes new substituted naphthyridines that inhibit the protein kinase Syk (spleen tyrosine kinase), the preparation and formulation thereof and itheir use for preparing a medicament.

Syk is an intracellular tyrosine kinase that has an important mediator function in the signal transduction of different receptors in B-cells, mast cells, monocytes, macrophages, neutrophils, T-cells, dendritic cells and epithelial cells. The receptors in which Syk performs an important function in signal transduction include for example the receptors for IgE (Fc\acute{ε}RI) and IgG (FcγR1) on mast cells and B cells, the B-cell receptor (BCR) and the T-cell receptor (TCR) on B- and T-cells, the ICAM1 receptor (ICAM1R) on epithelial cells of the respiratory tract, the DAP12-receptor on natural killer cells, dendritic cells and osteoclasts, the dectin 1-receptor on a subpopulation of T-helper cells (Th-17 cells), as well as the integrin receptors for β1-, β2- and β3-integrins on neutrophils, monocytes and macrophages (Wong et al.; Expert Opin. Investig. Drugs (2004) 13(7), 743-762; Ulanova et al.; Expert Opion. Ther. Target (2005) 9(5); 901-921; Wang et al.; J. Immunol. (2006) 177, 6859-6870; LeibundGut-Landmann et al.; Nature Immunology (2007) 8, 630-638; Slack et al., European J. Immunol. (2007) 37, 1600-1612). The best description is of the molecular processes during the signal transduction of the Fc\acute{ε}RI. In mast cells the binding of IgE to Fc\acute{ε}RI causes the cross-linking of IgE-receptors and the recruiting and activation of Lyn (a tyrosine kinase from the Src family). Active Lyn phoshorylates so-called ITAM motifs, which are present in may of the receptors listed above, and thereby generates binding sites for the SH2-domain of Syk. As a result of the binding to the ITAM motif Syk is activated and then phosphorylates various substrates which are needed for the release of allergic and inflammatory mediators such as e.g. histamine and β-hexosamidase (βHA), as well as for the synthesis of lipid mediators, such as e.g. prostaglandins and leukotrienes.

In view of its central function in different signal transduction pathways Syk has been discussed as a therapeutic target for different diseases such as e.g. Allergic rhinitis, asthma, autoimmune diseases, rheumatoid arthritis, osteopenia, osteoporosis, COPD and various leukaemias and lymphomas (Wong et al.; Expert Opin. Investig. Drugs (2004) 13(7), 743-762; Ulanova et al.; Expert Opion. Ther. Target (2005) 9(5); 901-921; Sigh and Masuda. Annual Reports in Medicinal Chemistry (2007) Vol 42; 379-391; Bajpai et al.; Expert Opin. Investig. Drugs (2008) Vol 15 (5); 641-659; Masuda and Schmitz; PPT (2008) Vol 21; 461-467).

Allergic rhinitis and asthma are diseases associated with allergic reactions and inflammatory processes and involving different cell types such as e.g. Mast cells, eosinophils, T-cells and dendritic cells. After exposure to allergens has occurred, the high affinity immunoglobulin receptors for IgE (Fc\acute{ε}RI) and IgG (FcγR1) are activated and induce the release of pro-inflammatory mediators and bronchoconstrictors. An inhibitor of the Syk kinase activity should thus be able to inhibit these steps.

Rheumatoid arthritis (RA) is an autoimmune disease in which the bones and ligaments structures surrounding the joints are progressively destroyed. In the pathophysiology of RA, B-cells play a significant role, as has been demonstrated for example by the therapeutic use of rituximab, a B cell-depleting antibody. In addition to the function of Syk in the signal transduction of the BCR (which after being stimulated also induces the release of pro-inflammatory mediators), Syk also plays an important part in the maturation and proliferation of B cells (Cheng et al. Nature (1995) 378, 303-306, Cornall et al., PNAS (2000) 97(4), 1713-1718). An inhibitor of the Syk kinase activity may thus offer a therapeutic option for the treatment of autoimmune diseases such as RA and diseases with an increased proliferation of B cells, such as e.g. B-cell lymphomas.

Chronic obstructive pulmonary disease (COPD) is characterised by a successive deterioration in lung function and chronic inflammation of the airways, which is initiated and produced by noxious substances of all kinds and contributes to the maintenance of the course of the disease. At a cellular level, in COPD there is in particular a multiplication of T-lymphocytes, neutrophils, granulocytes and macrophages. In particular, there is an increase in the number of CD8-positive lymphocytes, that is directly connected with the impairment of lung function. Another characteristic of COPD are acute deteriorations in lung function (exacerbations), characterised by viral (e.g. Rhinovirus), or bacterial (e.g. Streptococcus pneumoniae, Haemophilus influenzae and Moraxella catarrhalis) infections.

In view of the pro-inflammatory function of Syk in macrophages, T-cells and neutrophils as described above (see: Wong et al.; Expert Opin. Investig. Drugs (2004) 13(7), 743-762; and references cited therein) an inhibitor of the Syk kinase activity could be a new therapeutic approach to the treatment of the inflammatory processes that underlie COPD. It has also been shown that Syk in epithelial cells of the respiratory tract is involved in the ICAM1R-mediated uptake and subsequent replication of the Rhinovirus and that a si-RNA against Syk blocks these steps (Wang et al.; J. Immunol. (2006) 177, 6859-6870; Lau et al.; J. Immunol. (2008) 180, 870-880). Thus, an inhibitor of the Syk kinase activity could also be used therapeutically in exacerbations caused by Rhinoviruses.

Various studies suggest that Syk is involved in the malignant transformation of lymphocytes (summarised in Sigh and Masuda. Annual Reports in Medicinal Chemistry (2007) Vol 42; 379-391). A TEL-Syk fusion protein with a constitutive Syk activity transformed B cells of a patient with myelodysplastic syndrome, a constitutively active ITK-Syk fusion protein was isolated from patients with T-cell lymphomas. Moreover, constitutively active Syk was found in B-cell lymphoma cells of patients. On the basis of these data it seems that Syk is a proto-oncogene in haematopoietic cells and represents a potential target for the treatment of certain leukaemias and lymphomas.

### 1.2 Prior Art

BE 835770 describes 5-amino-1,6-naphthyridine with an antimicrobial activity. U.S. Pat. No. 3,928,367, U.S. Pat. No. 4,017,500, U.S. Pat. No. 4,115,395 and U.S. Pat. No. 4,260,759 describe 5-amino-1,6-naphthyridines with an antifungal and antibacterial activity. WO 9918077 describes 5-piperazinyl-1,6-naphthyridines as serotonin antagonists. U.S. Pat. No. 7,321,041 describes substituted [1,6]-naphthyridines as SYK-inhibitors, although they have a completely different substitution pattern from the compounds according to the invention.

### 2. DESCRIPTION OF THE INVENTION

Surprisingly it has now been found that naphthyridines of formula 1 are particularly suitable for the treatment of respiratory complaints, allergic diseases, osteoporosis, gastrointestinal diseases, autoimmune diseases, inflammatory diseases and diseases of the peripheral or central nervous system, particularly for the treatment of asthma, allergic rhinitis, rheumatoid arthritis, allergic dermatitis and COPD.

The present invention therefore relates to compounds of formula 1,

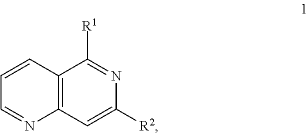

wherein
R¹ is a group A selected from among —O—R³, —NR³R⁴, —CR³R⁴R⁵, -(ethyne)-R³, —S—R³, —SO—R³ and SO₂—R³
or
R¹ is a group B selected from among
C₆₋₁₀-aryl,
five- to ten-membered, mono- or bicyclic heteroaryl with 1-3 heteroatoms selected independently of one another from among N, O and S; while this heteroaryl is linked to the structure according to formula 1 via either a C atom or an N atom,
three- to ten-membered, mono- or bicyclic, saturated or partially saturated heterocyclic group with 1-3 heteroatoms selected independently of one another from among N, O and S, while this heterocyclic group is linked to the structure according to formula 1 via either a C atom or an N atom,
   and
5- to 11-membered spiro group which may optionally contain 1, 2 or 3 heteroatoms selected independently of one another from among N, O and S, while this spiro group is linked to the structure according to formula 1 via either a C atom or an N atom,
   wherein this group B may optionally be substituted by one or more groups selected independently of one another from among H, halogen, —C₁₋₃-alkyl, —NH(C₁₋₄-alkyl), —N(C₁₋₄-alkyl)₂, —NH₂, —C₁₋₃-alkyl-OH, —OH, oxo, —CO—NH₂, —C₁₋₃-alkylene-CO—NH₂, —CO—NH—(C₁₋₃-alkyl), —C₁₋₃-alkylene-CO—NH(C₁₋₃-alkyl), —CO—NH(C₃₋₆-cycloalkyl), —C₁₋₃-alkylene-CO—NH(C₃₋₆-cycloalkyl), —NH—CO—NH₂, —NH—CO—NH(C₁₋₃-alkyl), —NH—CO—N(C₁₋₃-alkyl)₂, O—C₁₋₃-alkyl, —(C₁₋₃-alkylene)-NH₂, -phenyl and —CO—(C₁₋₅-alkyl),
   wherein
   R² denotes

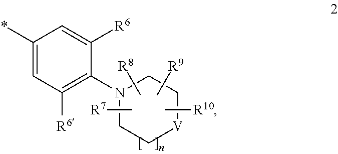

wherein
V denotes CH₂, O, NH, S, SO, SO₂, N—(C₁₋₃-alkyl), N—(C₁₋₃-alkylene)-(C₃₋₇-cycloalkyl), N—(C₃₋₇-cycloalkyl), N—CO—C₁₋₆-alkyl, N—CO—(C₃₋₇-cycloalkyl), N—(C₁₋₃-alkylene)-phenyl
n=0-2
R⁶ and R^{6′} are selected independently of one another from among
H, halogen, methyl, —O-methyl, ethyl, —O-ethyl, propyl, —O-propyl, OH, ═O,
—CO—NH₂, —CO—NH—C₁₋₃-alkyl, —COON, —COO—C₁₋₃-alkyl
   R⁷; R⁸, R⁹ and R¹⁹ denote H, C₁₋₃-alkyl, —O—(C₁₋₃-alkyl), F, ═O or OH,
   R³ denotes H
   or a group selected from among —C₁₋₆-alkyl, —C₁₋₆-fluoroalkyl,
   —(C₁₋₅-alkyl)—OH, —C₆₋₁₀-aryl, —C₁₋₄-alkylene-C₆₋₁₀-aryl, -ethenyl, —C₁₋₄-alkylene-(ethene), -ethynyl, —C₁₋₄-alkylene-(ethyne), —C₁₋₄-alkylene-(ethyne)-NH₂, —C₁₋₄-alkylene-(ethyne)-(C₁₋₄-alkylene)-NH₂, —CHOH—(C₁₋₄-alkylene)-NH₂, —(C₁₋₄-alkylene)-CHOH—(C₁₋₄-alkylene)-NH₂, —CHOH—NH₂, —(C₁₋₄-alkylene)-CHOH—NH₂, —NH(C₁₋₃-alkylene), —(C₁₋₄-alkylene)-NH(C₁₋₃-alkyl), mono- or bicyclic, saturated or partially saturated —C₃₋₁₀-cycloalkyl, mono- or bicyclic, saturated or partially saturated —(C₁₋₄-alkylene)-C₃₋₁₀-cycloalkyl, -(het), —(C₁₋₄-alkylene)-(het), -(hetaryl), and —(C₁₋₄-alkylene)-(hetaryl),
   while this group may optionally be substituted by one or more groups selected independently of one another from among H, —OH, -oxo, —COON, -halogen, —C₁₋₃-alkyl, —C₁₋₃-haloalkyl, —C₁₋₃-alkyl-OH, —C₃₋₇-cycloalkyl, —O—(C₁₋₄-alkyl), —NH(C₁₋₄-alkyl), —(C₁₋₄-alkylene)-NH(C₁₋₄-alkyl), —N(C₁₋₄-alkyl)₂, —(C₁₋₄-alkylene)-N(C₁₋₄-alkyl)₂, —NH—CO—NH₂, —(C₁₋₄-alkylene)-NH—CO—NH₂, —CO—NH₂, —(C₁₋₄-alkylene)-CO—NH₂, —CO—NH(C₁₋₃-alkyl), —(C₁₋₄-alkylene)-CO—NH(C₁₋₃-alkyl), —CO—N(C₁₋₃-alkyl)₂, —(C₁₋₄-alkylene)-CO—N(C₁₋₃-alkyl)₂, —NH—(CO)ₘ—NH₂, —NH—(C₁₋₄-alkylene)-(CO)ₘ—NH₂, —NH—(CO)ₘ—NH(C₁₋₃-alkyl), —NH—(C₁₋₄-alkylene)-(CO)ₘ—NH(C₁₋₃-alkyl), —NH—(CO)ₘ—N(C₁₋₃-alkyl)₂, —NH—(C₁₋₄-alkylene)-(CO)ₘ—N(C₁₋₃-alkyl)₂, —O—(C₂₋₄-alkylene)-NH₂, —O—(C₂₋₄-alkylene)-NH(C₁₋₃-alkyl), —O—(C₂₋₄-alkylene)-N(C₁₋₃-alkyl)₂, —NH—CO—(C₁₋₃-alkyl), —(C₁₋₄-alkylene)-NH—CO—(C₁₋₃-alkyl), —C₃₋₅-cycloalkyl, —SO₂—(C₁₋₄-alkyl), —SO₂—(C₃₋₅-cycloalkyl), —SO₂—NH₂, —SO₂—NH—C₁₋₃-alkyl, —SO₂—N(C₁₋₃-alkyl)₂, —SO₂-(het), —O-(het), —O—(C₁₋₄-alkylene)-(het), —NH-(het), —NH—(C₁₋₄-alkylene)-(het), —NH-(hetaryl), —NH—(C₁₋₄-alkylene)-(hetaryl), -(het) and —(C₁₋₄-alkylene)-(het),
   wherein (het) denotes a three- to ten-membered, saturated or partially saturated, mono- or bicyclic, heterocyclic group optionally substituted by 1-3 groups selected from C₁₋₃-alkyl, halogen, CH₂—NH₂, NH₂, OH; CO—NH₂ and oxo, which contains 1-3 heteroatoms selected independently of one another from among N, O and S,
   and
   wherein (hetaryl) denotes a five- to ten-membered, mono- or bicyclic, heteroaryl optionally substituted by with 1-3 groups selected from C₁₋₃-alkyl, halogen, CH₂—NH₂, NH₂, OH, CO—NH₂ and oxo, which contains 1-3 heteroatoms selected independently of one another from among N, O and S,
   wherein
   m=0 or 1
   and
   R⁴ and R⁵ denote H, methyl or ethyl,
   and pharmaceutically acceptable salts, diastereomers, enantiomers, racemates, hydrates and solvates thereof.

A preferred object of the present invention relates to compounds of the above formula 1 with the above-mentioned definitions of the individual variables, wherein n=1, and pharmaceutically acceptable salts, diastereomers, enantiomers, racemates, hydrates and solvates thereof.

Also preferred are compounds of formula 1 with the above-mentioned definitions of the individual variables, wherein R⁶ and R^{6′} independently of one another are selected from among H, methyl and —OCH₃, and pharmaceutically acceptable salts, diastereomers, enantiomers, racemates, hydrates and solvates thereof.

The present invention preferable further relates to compounds of formula 1 with the above-mentioned definitions of the individual variables, wherein R⁷; R⁸, R⁹ and R¹⁹ are each independently of one another selected from among H or —OCH₃, and pharmaceutically acceptable salts, diastereomers, enantiomers, racemates, hydrates and solvates thereof.

In another preferred aspect the present invention relates to compounds of formula 1 with the above-mentioned definitions of the individual variables, wherein V denotes either N—CH₃, O or N—(C₁₋₃-alkylene)-phenyl, and pharmaceutically acceptable salts, diastereomers, enantiomers, racemates, hydrates and solvates thereof.

Also preferred are compounds of formula 1 with the above-mentioned definitions of the individual variables, wherein R¹ is selected from among —O—R³, —NR³R⁴—CR³R⁴R⁵ and -(ethyne)-R³, and pharmaceutically acceptable salts, diastereomers, enantiomers, racemates, hydrates and solvates thereof.

In another preferred aspect the present invention relates to compounds of formula 1 with the above-mentioned definitions of the individual variables, wherein

R¹ denotes —NR³R⁴,
R⁴ denotes H
and R³ is selected from among —C₆₋₁₀-aryl, —C₁₋₄-alkylene-C₆₋₁₀-aryl, -(het), —(C₁₋₄-alkylene)-(het), -(hetaryl), and —(C₁₋₄-alkylene)-(hetaryl),
wherein this group R³ may optionally be substituted by one or more groups selected independently of one another from among H, —OH, -oxo, —COON, —C₁₋₃-alkyl, —C₁₋₃-haloalkyl, —C₁₋₃-alkyl-OH, —CO—NH₂, —(C₁₋₄-alkylene)-CO—NH₂, —CO—NH(C₁₋₃-alkyl), —(C₁₋₄-alkylene)-CO—NH(C₁₋₃-alkyl), —CO—N(C₁₋₃-alkyl)₂, —(C₁₋₄-alkylene)-CO—N(C₁₋₃-alkyl)₂, —NH—(CO)ₘ—NH₂, —NH—(C₁₋₄-alkylene)-(CO)ₘ—NH₂, —NH—(CO)ₘ—NH(C₁₋₃-alkyl), —NH—(C₁₋₄-alkylene)-(CO)ₘ—NH(C₁₋₃-alkyl), —NH—(CO)ₘ—N(C₁₋₃-alkyl)₂ and —NH—(C₁₋₄-alkylene)-(CO)ₘ—N(C₁₋₃-alkyl)₂, and pharmaceutically acceptable salts, diastereomers, enantiomers, racemates, hydrates and solvates thereof.

Particularly preferred within the scope of the present invention are the compounds of formula 1 with the above-mentioned definitions of the individual variables, wherein

R¹ denotes —NR³R⁴ and
R⁴ denotes H
and R³ is selected from among —C₆₋₁₀-aryl, —C₁₋₄-alkylene-C₆₋₁₀-aryl, -(het), —(C₁₋₄-alkylene)-(het), -(hetaryl), and —(C₁₋₄-alkylene)-(hetaryl),
wherein this group R³ may optionally be substituted by one or more groups selected independently of one another from among H, —OH, -oxo, —COON, —C₁₋₃-alkyl, —CO—NH₂, —(C₁₋₄-alkylene)-CO—NH₂, —CO—NH(C₁₋₃-alkyl), —(C₁₋₄-alkylene)-CO—NH(C₁₋₃-alkyl), —CO—N(C₁₋₃-alkyl)₂, —(C₁₋₄-alkylene)-CO—N(C₁₋₃-alkyl)₂, and pharmaceutically acceptable salts, diastereomers, enantiomers, racemates, hydrates and solvates thereof.

In another preferred aspect the present invention relates to compounds of formula 1 with the above-mentioned definitions of the individual variables, wherein

R¹ denotes —OR³ and
R⁴ denotes H,
and wherein R³ is selected from among —C₆₋₁₀-aryl, —C₁₋₄-alkylene-C₆₋₁₀-aryl, -(het), -(C₁₋₄-alkylene)-(het), -(hetaryl), and —(C₁₋₄-alkylene)-(hetaryl),
wherein this group R³ may optionally be substituted by one or more groups selected independently of one another from among H, —OH, -oxo, —COON, —C₁₋₃-alkyl, —C₁₋₃-haloalkyl, —C₁₋₃-alkyl-OH, —CO—NH₂, —(C₁₋₄-alkylene)-CO—NH₂, —CO—NH(C₁₋₃-alkyl), —(C₁₋₄-alkylene)-CO—NH(C₁₋₃-alkyl), —CO—N(C₁₋₃-alkyl)₂, —(C₁₋₄-alkylene)-CO—N(C₁₋₃-alkyl)₂, —NH—(CO)ₘ—NH₂, —NH—(C₁₋₄-alkylene)-(CO)ₘ—NH₂, —NH—(CO)ₘ—NH(C₁₋₃-alkyl), —NH—(C₁₋₄-alkylene)-(CO)ₘ—NH(C₁₋₃-alkyl), —NH—(CO)ₘ—N(C₁₋₃-alkyl)₂ and —NH—(C₁₋₄-alkylene)-(CO)ₘ—N(C₁₋₃-alkyl)₂, and pharmaceutically acceptable salts, diastereomers, enantiomers, racemates, hydrates and solvates thereof.

In another preferred aspect the present invention relates to compounds of formula 1 with the above-mentioned definitions of the individual variables, wherein

R¹ denotes —CR³R⁴R⁵,
R⁴ denotes H, methyl
and
R⁵ denotes H, methyl,
and wherein R³ is selected from among —C₆₋₁₀-aryl, —C₁₋₄-alkylene-C₆₋₁₀-aryl, -(het), —(C₁₋₄-alkylene)-(het), -(hetaryl), and —(C₁₋₄-alkylene)-(hetaryl), wherein this group R³ may optionally be substituted by one or more groups selected independently of one another from among H, —OH, -oxo, —COON, —C₁₋₃-alkyl, —C₁₋₃-haloalkyl, —C₁₋₃-alkyl-OH, —CO—NH₂, —(C₁₋₄-alkylene)-CO—NH₂, —CO—NH(C₁₋₃-alkyl), —(C₁₋₄-alkylene)-CO—NH(C₁₋₃-alkyl), —CO—N(C₁₋₃-alkyl)₂, —(C₁₋₄-alkylene)-CO—N(C₁₋₃-alkyl)₂, —NH—(CO)ₘ—NH₂, —NH—(C₁₋₄-alkylene)-(CO)ₘ—NH₂, —NH—(CO)ₘ—NH(C₁₋₃-alkyl), —NH—(C₁₋₄-alkylene)-(CO)ₘ—NH(C₁₋₃-alkyl), —NH—(CO)ₘ—N(C₁₋₃-alkyl)₂ and —NH—(C₁₋₄-alkylene)-(CO)ₘ—N(C₁₋₃-alkyl)₂, and pharmaceutically acceptable salts, diastereomers, enantiomers, racemates, hydrates and solvates thereof.

The invention also relates particularly preferably to compounds of formula 1 with the above-mentioned definitions of the individual variables, wherein R¹ is selected from among
five- to ten-membered, mono- or bicyclic heteroaryl with 1-3 heteroatoms selected independently of one another from among N, O and S; wherein at least of one of the 1-3 heteroatoms is an N atom and
three- to ten-membered, mono- or bicyclic, saturated or partially saturated heterocyclic group with 1-3 heteroatoms selected independently of one another from among N, O and S, wherein at least of one of the 1-3 heteroatoms is an N atom,
wherein the above-mentioned heteroaryls and heterocycles are each linked via the at least one N atom to the structure according to formula 1,
or wherein R¹ is a
5- to 11-membered spiro group which contains 1, 2 or 3 heteroatoms selected independently of one another from among N, O and S, wherein at least one of the 1-3 heteroatoms of this spiro group is an N atom and wherein the spiro group is linked to the structure according to formula 1 via this N atom, and pharmaceutically acceptable salts, diastereomers, enantiomers, racemates, hydrates and solvates thereof.

In particular the present invention relates to compounds of formula 1 with the above-mentioned definitions of the individual variables,

wherein R¹ is selected from among

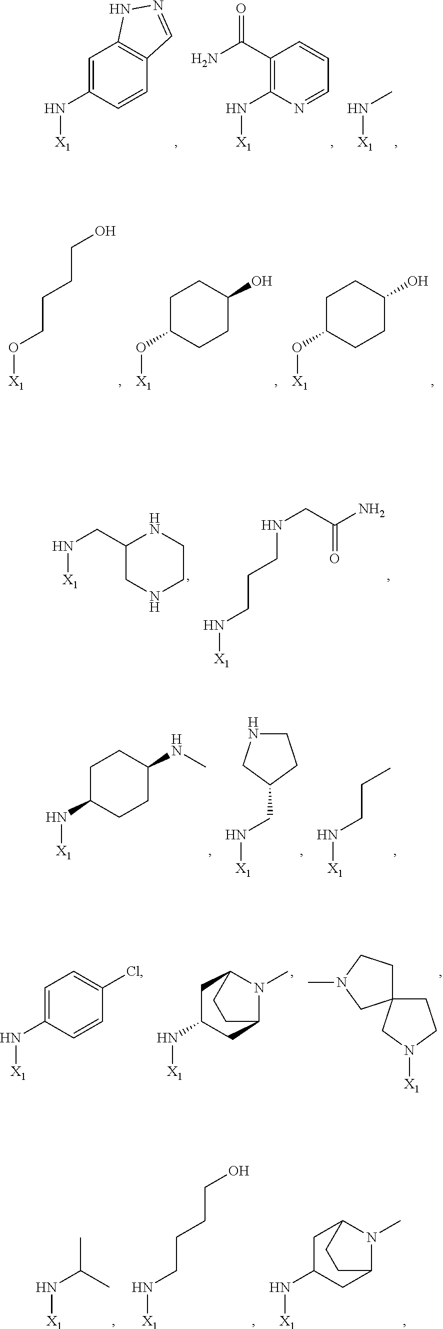

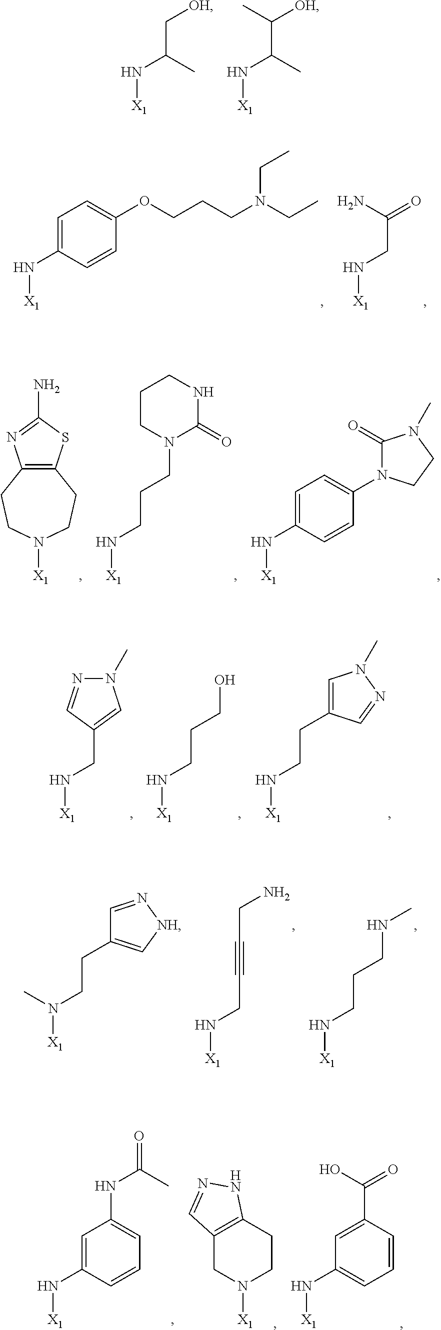

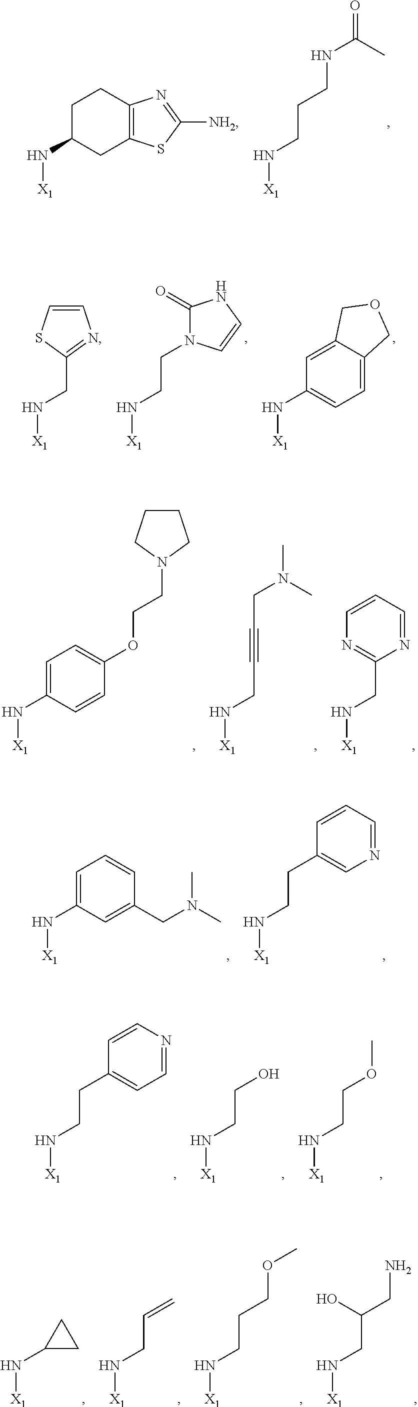

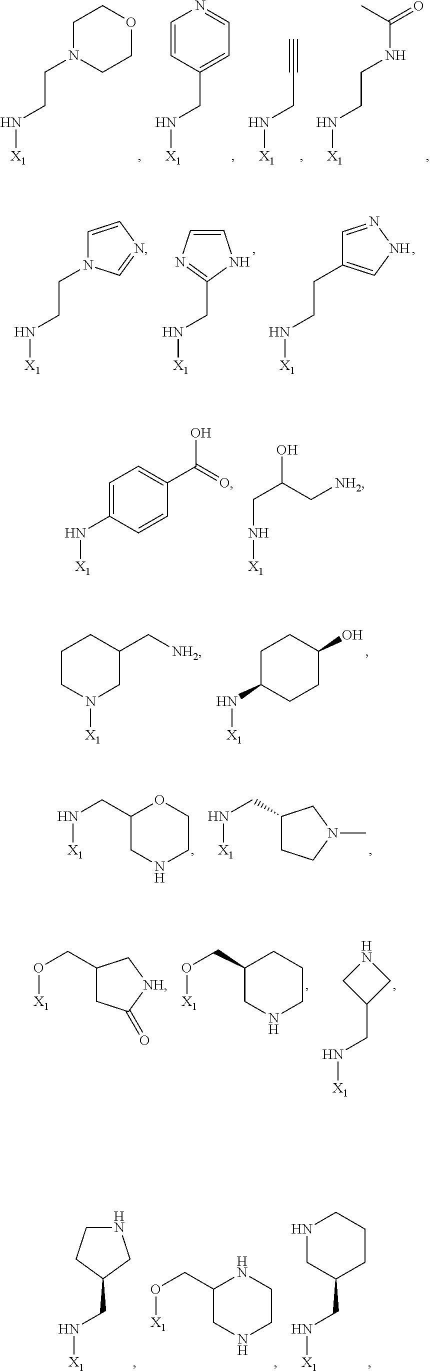

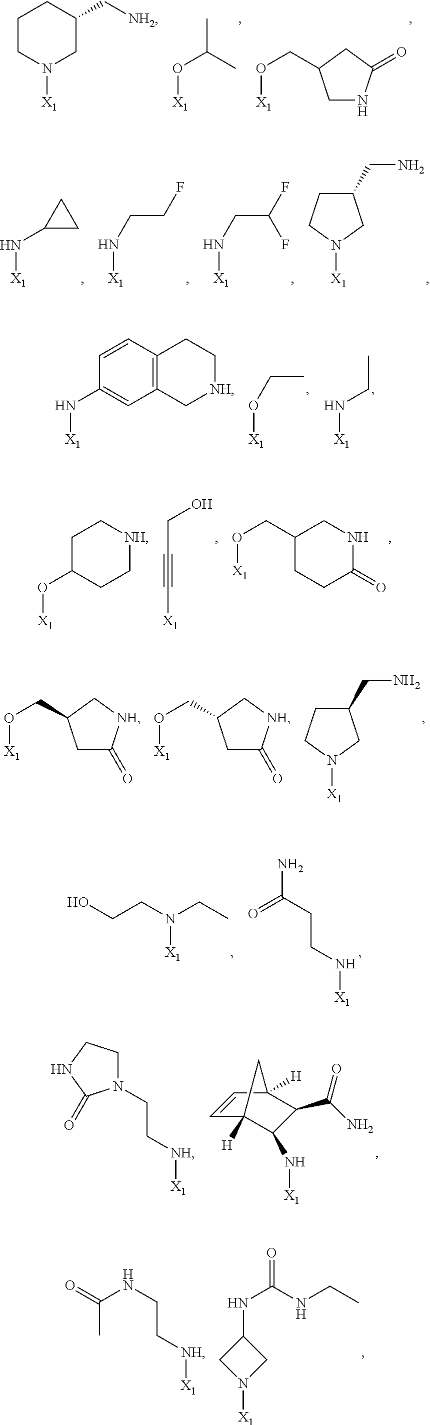

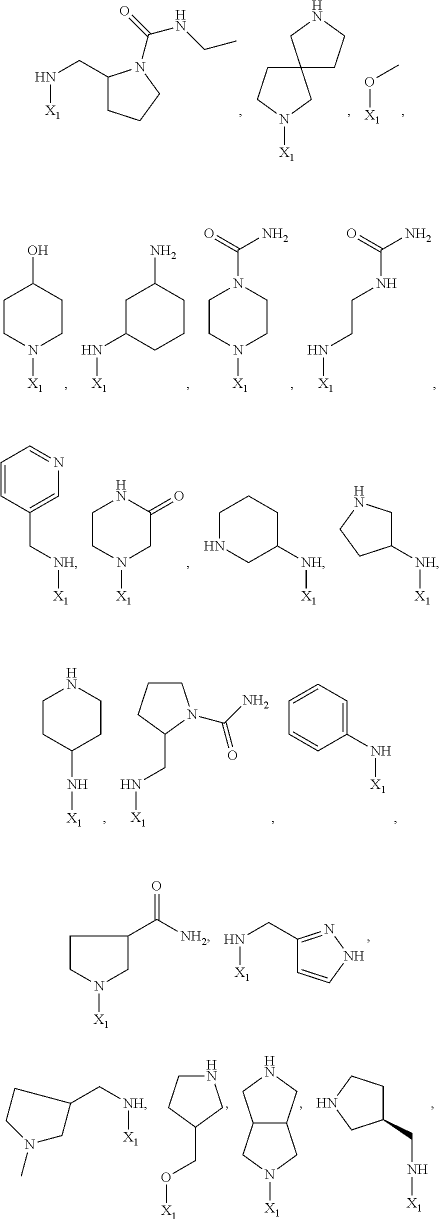

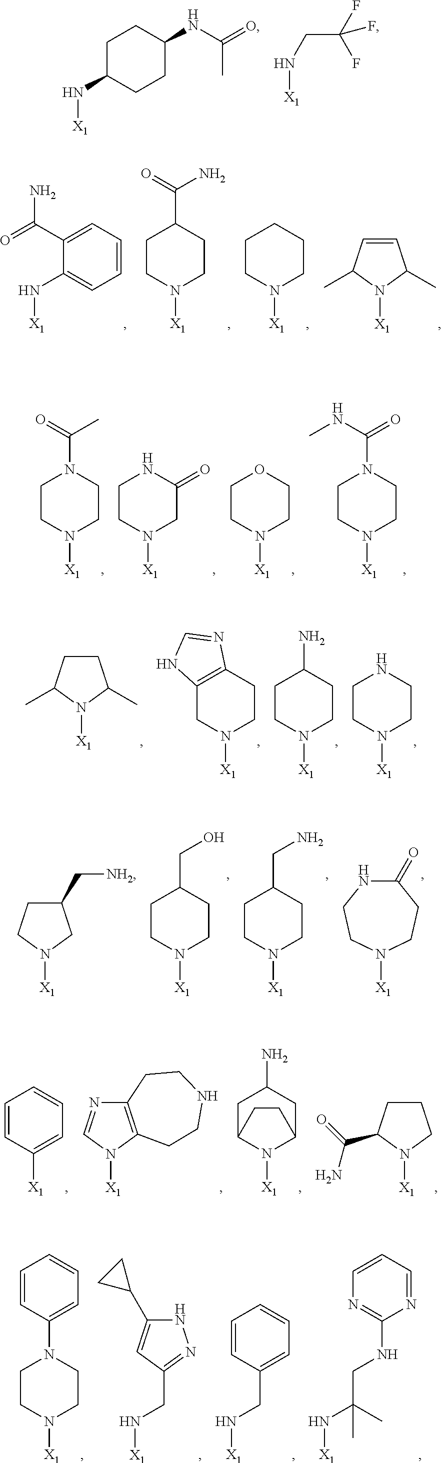

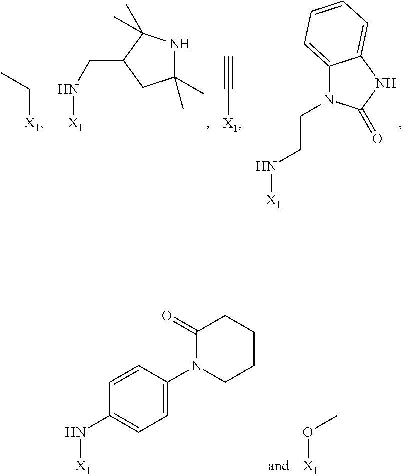

wherein R² is selected from among

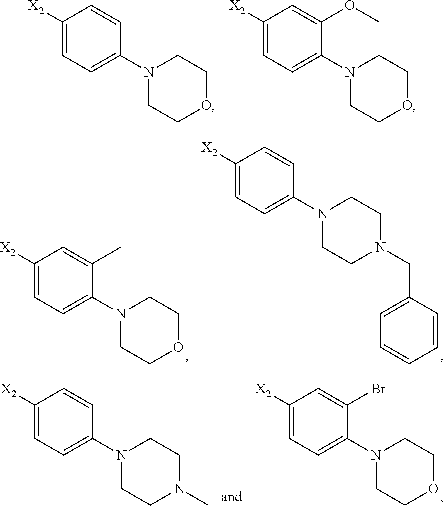

wherein X₁ denotes the point of attachment of R¹ to the structure of formula 1 and X₂ denotes the point of attachment of R² to the structure of formula 1, and pharmaceutically acceptable salts, diastereomers, enantiomers, racemates, hydrates and solvates thereof.

The invention further relates to the above compounds of formula 1 with the above-mentioned definitions of the individual variables as pharmaceutical compositions.

The invention further relates to the use of the above compounds of formula 1 with the above-mentioned definitions of the individual variables for preparing a medicament for the treatment of diseases treated by inhibiting the SYK enzyme.

In another preferred aspect the invention relates to the use of the above compounds of formula 1 with the above-mentioned definitions of the individual variables for preparing a medicament for the treatment of diseases selected from among allergic rhinitis, asthma, COPD, adult respiratory distress syndrome, bronchitis, dermatitis and contact dermatitis, allergic dermatitis, allergic rhinoconjunctivitis, rheumatoid arthritis, anti-phospholipid syndrome, Berger's disease, Evans's syndrome, ulcerative colitis, allergic antibody-based glomerulonephritis, granulocytopenia, Goodpasture's syndrome, hepatitis, Henoch-Schönlein purpura, hypersensitivity vasculitis, immunohaemolytic anaemia, idiopathic thrombocytopenic purpura, Kawasaki syndrome, allergic conjunctivitis, lupus erythematodes, neutropenia, non-familial lateral sclerosis, Crohn's disease, multiple sclerosis, myasthenia gravis, osteoporosis, osteolytic diseases, osteopenia, psoriasis, Sjögren's syndrome, sclerodermy, urticaria/angiooedema, Wegener's granulomatosis and coeliac disease.

In a particularly preferred aspect the present invention relates to the use of the above compounds of formula 1 with the above-mentioned definitions of the individual variables for preparing a medicament for the treatment of diseases selected from among asthma, COPD, allergic rhinitis, adult respiratory distress syndrome, bronchitis, allergic dermatitis, contact dermatitis, idiopathic thrombocytopenic purpura, rheumatoid arthritis and allergic rhinoconjunctivitis.

The present invention relates in particular to the use of the above compounds of formula 1 with the above-mentioned definitions of the individual variables for preparing a medicament for the treatment of diseases selected from among asthma, COPD, allergic rhinitis, allergic dermatitis and rheumatoid arthritis.

Moreover the present invention preferably relates to pharmaceutical formulations which contain one or more compounds of formula 1 with the above-mentioned definitions of the individual variables.

The invention further relates to pharmaceutical formulations which contain one or more compounds of formula 1 with the above-mentioned definitions of the individual variables, in combination with an active substance selected from among the betamimetics, corticosteroids, PDE4-inhibitors, EGFR-inhibitors and LTD4-antagonists, CCR3-inhibitors, iNOS-inhibitors and other SYK-inhibitors.

In another preferred aspect the invention relates to the following intermediate products in the preparation of the above compounds according to formula 1 selected from among

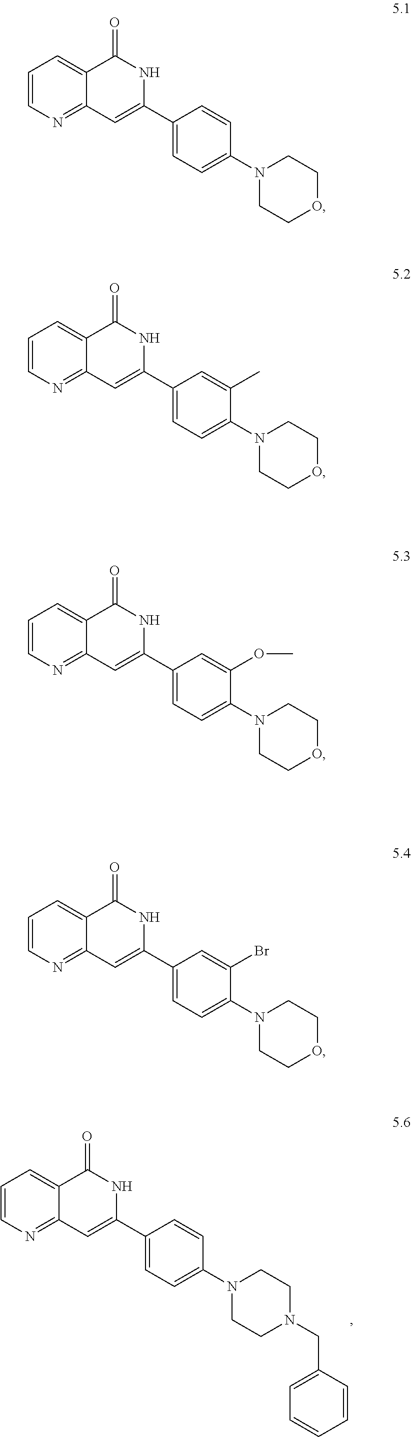

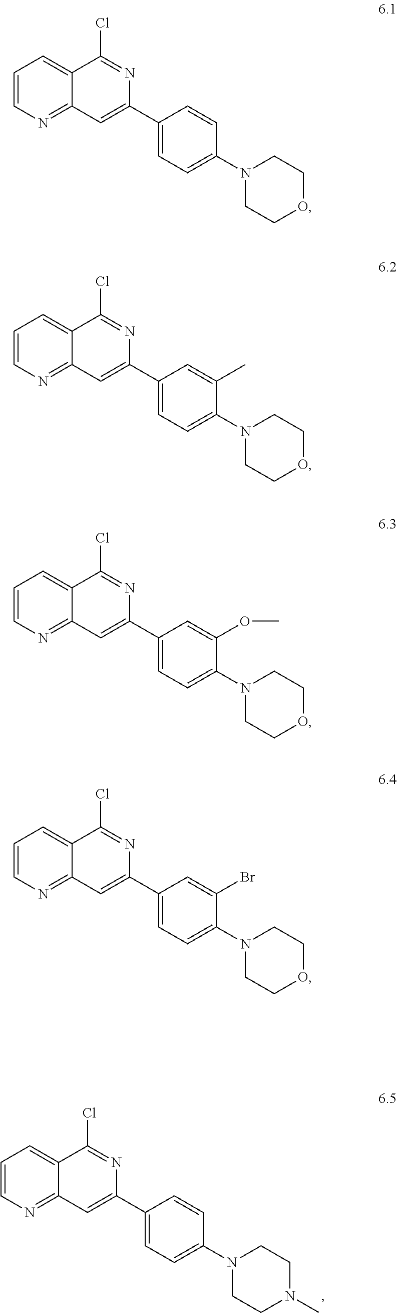

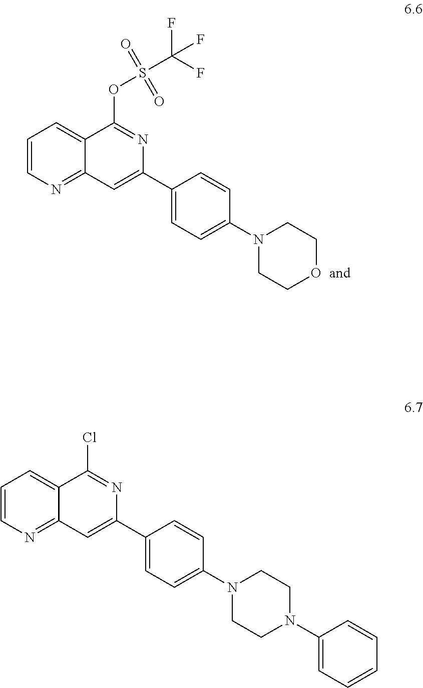

and pharmaceutically acceptable salts, diastereomers, enantiomers, racemates, hydrates and solvates thereof.

### 3. TERMS AND DEFINITIONS USED

Unless stated otherwise, all the substituents are independent of one another. If for example a number of C₁₋₆-alkyl groups are possible substituents at a group, in the case of three substituents, for example, C₁₋₆-alkyl could represent, independently of one another, a methyl, an n-propyl and a tert-butyl.

Within the scope of this application, in the definition of possible substituents, these may also be presented in the form of a structural formula. An asterisk (*) in the structural formula of the substituent is to be understood as being the linking point to the rest of the molecule. Mor3eover, the atom of the substituent following the linking point is understood as being the atom in position number 1. Thus for example the groups N-piperidinyl (I), 4-piperidinyl (II), 2-tolyl (III), 3-tolyl (IV) and 4-tolyl (V) are represented as follows:

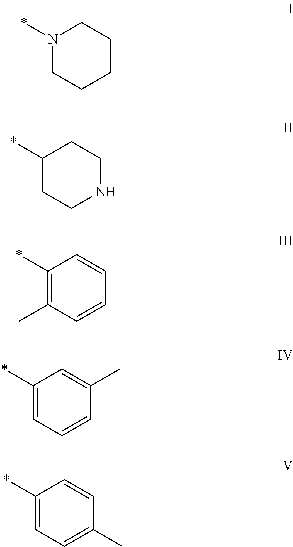

If there is no asterisk (*) in the structural formula of the substituent, each hydrogen atom may be removed at the substituent and the valency thus freed may serve as a binding site to the rest of a molecule. Thus, for example, VI

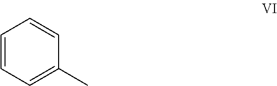

may represent 2-tolyl, 3-tolyl, 4-tolyl and benzyl.

Alternatively to the * within the scope of this application X₁ is also understood as being the linking point of the group R¹ to the structure of formula 1 and X₂ as being the linking point of the group R² to the structure of formula 1.

By the term “C₁₋₆-alkyl” (including those which are part of other groups) are meant branched and unbranched alkyl groups with 1 to 6 carbon atoms and by the term “C₁₋₃-alkyl” are meant branched and unbranched alkyl groups with 1 to 3 carbon atoms. “C₁₋₄-alkyl” accordingly denotes branched and unbranched alkyl groups with 1 to 4 carbon atoms. Alkyl groups with 1 to 4 carbon atoms are preferred.

Examples of these include: methyl, ethyl, n-propyl, iso-propyl, n-butyl, iso-butyl, sec-butyl, tert-butyl, n-pentyl, iso-pentyl, neo-pentyl or hexyl. The abbreviations Me, Et, n-Pr, i-Pr, n-Bu, i-Bu, t-Bu, etc., may also optionally be used for the above-mentioned groups. Unless stated otherwise, the definitions propyl, butyl, pentyl and hexyl include all the possible isomeric forms of the groups in question. Thus, for example, propyl includes n-propyl and iso-propyl, butyl includes iso-butyl, sec-butyl and tert-butyl etc.

By the term “C₁₋₆-alkylene” (including those which are part of other groups) are meant branched and unbranched alkylene groups with 1 to 6 carbon atoms and by the term “C₁₋₄-alkylene” are meant branched and unbranched alkylene groups with 1 to 4 carbon atoms. Alkylene groups with 1 to 4 carbon atoms are preferred. Examples of these include: methylene, ethylene, propylene, 1-methylethylene, butylene, 1-methylpropylene, 1,1-dimethylethylene, 1,2-dimethylethylene, pentylene, 1,1-dimethylpropylene, 2,2-dimethylpropylene, 1,2-dimethylpropylene, 1,3-dimethylpropylene or hexylene. Unless stated otherwise, the definitions propylene, butylene, pentylene and hexylene include all the possible isomeric forms of the groups in question with the same number of carbons. Thus, for example, propyl includes also 1-methylethylene and butylene includes 1-methylpropylene, 1,1-dimethylethylene, 1,2-dimethylethylene.

If the carbon chain is substituted by a group which together with one or two carbon atoms of the alkylene chain forms a carbocyclic ring with 3, 5 or 6 carbon atoms, this includes, inter alia, the following examples of the rings:

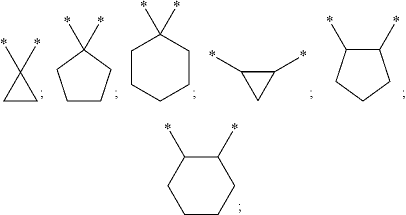

By the term “C₂₋₆-alkenyl” (including those which are part of other groups) are meant branched and unbranched alkenyl groups with 2 to 6 carbon atoms and by the term “C₂₋₄-alkenyl” are meant branched and unbranched alkenyl groups with 2 to 4 carbon atoms, provided that they have at least one double bond. Alkenyl groups with 2 to 4 carbon atoms are preferred. Examples include: ethenyl or vinyl, propenyl, butenyl, pentenyl or hexenyl. Unless stated otherwise, the definitions propenyl, butenyl, pentenyl and hexenyl include all the possible isomeric forms of the groups in question. Thus, for example, propenyl includes 1-propenyl and 2-propenyl, butenyl includes 1-, 2- and 3-butenyl, 1-methyl-1-propenyl, 1-methyl-2-propenyl etc.

By the term “C₂₋₆-alkenylene” (including those which are part of other groups) are meant branched and unbranched alkenylene groups with 2 to 6 carbon atoms and by the term “C₂₋₄-alkenylene” are meant branched and unbranched alkylene groups with 2 to 4 carbon atoms. Alkenylene groups with 2 to 4 carbon atoms are preferred. Examples of these include: ethenylene, propenylene, 1-methylethenylene, butenylene, 1-methylpropenylene, 1,1-dimethylethenylene, 1,2-dimethylethenylene, pentenylene, 1,1-dimethylpropenylene, 2,2-dimethylpropenylene, 1,2-dimethylpropenylene, 1,3-dimethylpropenylene or hexenylene. Unless stated otherwise, the definitions propenylene, butenylene, pentenylene and hexenylene include all the possible isomeric forms of the groups in question with the same number of carbons. Thus, for example, propenyl also includes 1-methylethenylene and butenylene includes 1-methylpropenylene, 1,1-dimethylethenylene, 1,2-dimethylethenylene.

By the term “C₂₋₆-alkynyl” (including those which are part of other groups) are meant branched and unbranched alkynyl groups with 2 to 6 carbon atoms and by the term “C₂₋₄-alkynyl” are meant branched and unbranched alkynyl groups with 2 to 4 carbon atoms, provided that they have at least one triple bond. Alkynyl groups with 2 to 4 carbon atoms are preferred. Examples include: ethynyl, propynyl, butynyl, pentynyl, or hexynyl. Unless stated otherwise, the definitions propynyl, butynyl, pentynyl and hexynyl include all the possible isomeric forms of the groups in question. Thus for example propynyl includes 1-propynyl and 2-propynyl, butynyl includes 1,2- and 3-butynyl, 1-methyl-1-propynyl, 1-methyl-2-propynyl etc.

By the term “C₂₋₆-alkynylene” (including those which are part of other groups) are meant branched and unbranched alkynylene groups with 2 to 6 carbon atoms and by the term “C₂₋₄-alkynylene” are meant branched and unbranched alkylene groups with 2 to 4 carbon atoms. Preferred are alkynylene groups with 2 to 4 carbon atoms. Examples include: ethynylene, propynylene, 1-methylethynylene, butynylene, 1-methylpropynylene, 1,1-dimethylethynylene, 1,2-dimethylethynylene, pentynylene, 1,1-dimethylpropynylene, 2,2-dimethylpropynylene, 1,2-dimethylpropynylene, 1,3-dimethylpropynylene or hexynylene. Unless stated otherwise, the definitions propynylene, butynylene, pentynylene and hexynylene include all the possible isomeric forms of the groups in question with the same number of carbons. Thus for example propynyl also includes 1-methylethynylene butynylene includes 1-methylpropynylene, 1,1-dimethylethynylene, 1,2-dimethylethynylene.

By the term “aryl” (including those which are part of other groups) are meant aromatic ring systems with 6 or 10 carbon atoms. Examples include: phenyl or naphthyl, the preferred aryl group being phenyl. Unless otherwise stated, the aromatic groups may be substituted by one or more groups selected from among methyl, ethyl, iso-propyl, tert-butyl, hydroxy, fluorine, chlorine, bromine and iodine.

By the term “aryl-C₁₋₆-alkylene” (including those which are part of other groups) are meant branched and unbranched alkylene groups with 1 to 6 carbon atoms, which are substituted by an aromatic ring system with 6 or 10 carbon atoms. Examples include: benzyl, 1- or 2-phenylethyl or 1- or 2-naphthylethyl. Unless otherwise stated, the aromatic groups may be substituted by one or more groups selected from among methyl, ethyl, iso-propyl, tert-butyl, hydroxy, fluorine, chlorine, bromine and iodine.

By the term “heteroaryl-C₁₋₆-alkylene” (including those which are part of other groups) are meant—even though they are already included under “aryl-C₁₋₆-alkylene”—branched and unbranched alkylene groups with 1 to 6 carbon atoms, which are substituted by a heteroaryl.

A heteroaryl of this kind includes five- or six-membered heterocyclic aromatic groups or 5-10-membered, bicyclic heteroaryl rings which may contain one, two, three or four heteroatoms selected from among oxygen, sulphur and nitrogen, and contain so many conjugated double bonds that an aromatic system is formed. The following are examples of five- or six-membered heterocyclic aromatic groups or bicyclic heteroaryl rings:

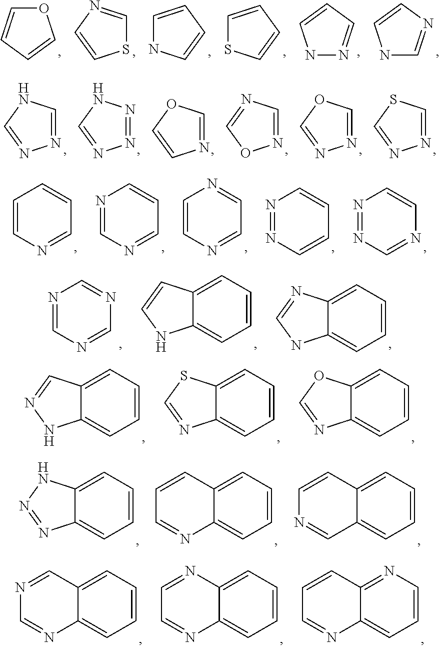

Unless otherwise stated, these heteroaryls may be substituted by one or more groups selected from among methyl, ethyl, iso-propyl, tert-butyl, hydroxy, fluorine, chlorine, bromine and iodine.

The following are examples of heteroaryl-C₁₋₆-alkylenes:

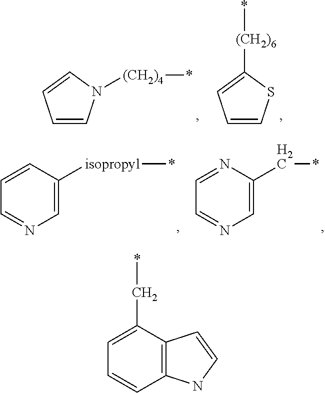

By the term “C₁₋₆-haloalkyl” (including those which are part of other groups) are meant branched and unbranched alkyl groups with 1 to 6 carbon atoms, which are substituted by one or more halogen atoms. By the term “C₁₋₄-alkyl” are meant branched and unbranched alkyl groups with 1 to 4 carbon atoms, which are substituted by one or more halogen atoms. Alkyl groups with 1 to 4 carbon atoms are preferred. Examples include: CF₃, CHF₂, CH₂F, CH₂CF₃.

By the term “C₃₋₇-cycloalkyl” (including those which are part of other groups) are meant cyclic alkyl groups with 3 to 7 carbon atoms. Examples include: cyclopropyl, cyclobutyl, cyclopentyl, cyclohexyl or cycloheptyl. Unless otherwise stated, the cyclic alkyl groups may be substituted by one or more groups selected from among methyl, ethyl, iso-propyl, tert-butyl, hydroxy, fluorine, chlorine, bromine and iodine.

By the term “C₃₋₁₀-cycloalkyl” are also meant monocyclic alkyl groups with 3 to 7 carbon atoms and also bicyclic alkyl groups with 7 to 10 carbon atoms, or monocyclic alkyl groups which are bridged by at least one C₁₋₃-carbon bridge.

By the term “heterocyclic rings” or “heterocycle” are meant, unless stated otherwise, five-, six- or seven-membered, saturated, partially saturated or unsaturated heterocyclic rings which may contain one, two or three heteroatoms, selected from among oxygen, sulphur and nitrogen, while the ring may be linked to the molecule through a carbon atom or through a nitrogen atom, if there is one. Although included by the term “heterocyclic rings” or “heterocycles”, the term “saturated heterocyclic ring” refers to five-, six- or seven-membered saturated rings. Examples include:

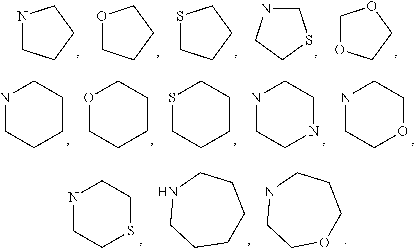

Although included by the term “heterocyclic rings” or “heterocyclic group”, the term “partially saturated heterocyclic group” refers to five-, six- or seven-membered partially saturated rings which contain one or two double bonds, without so many double bonds being produced that an aromatic system is formed. Examples include:

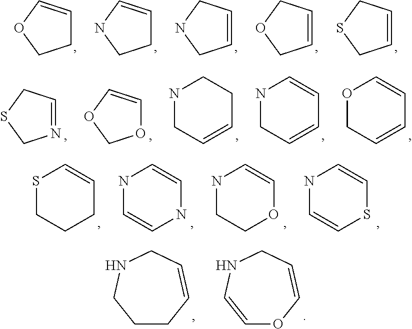

Although included by the term “heterocyclic rings” or “heterocycles”, the term “heterocyclic aromatic rings”, “unsaturated heterocyclic group” or “heteroaryl” refers to five- or six-membered heterocyclic aromatic groups or 5-10-membered, bicyclic heteroaryl rings which may contain one, two, three or four heteroatoms, selected from among oxygen, sulphur and nitrogen, and contain so many conjugated double bonds that an aromatic system is formed. Examples of five- or six-membered heterocyclic aromatic groups include:

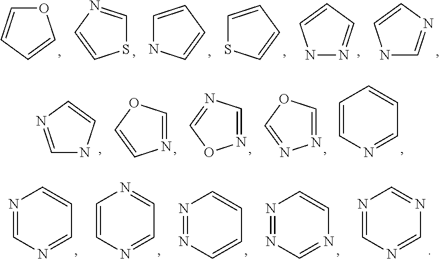

Unless otherwise mentioned, a heterocyclic ring (or heterocycle) may be provided with a keto group. Examples include:

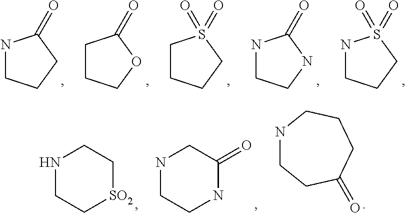

Although covered by the term “cycloalkyl”, the term “bicyclic cycloalkyls” generally denotes eight-, nine- or ten-membered bicyclic carbon rings. Examples include

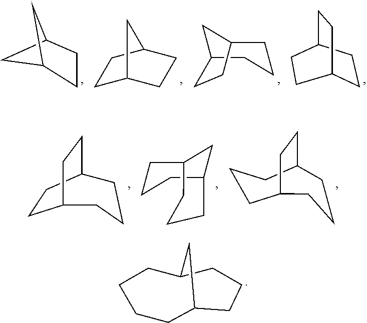

Although already included by the term “heterocycle”, the term “bicyclic heterocycles” generally denotes eight-, nine- or ten-membered bicyclic rings which may contain one or more heteroatoms, preferably 1-4, more preferably 1-3, even more preferably 1-2, particularly one heteroatom, selected from among oxygen, sulphur and nitrogen. The ring may be linked to the molecule through a carbon atom of the ring or through a nitrogen atom of the ring, if there is one. Examples include:

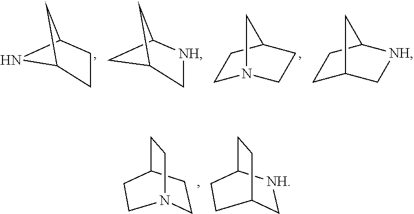

Although already included by the term “aryl”, the term “bicyclic aryl” denotes a 5-10 membered, bicyclic aryl ring which contains sufficient conjugated double bonds to form an aromatic system. One example of a bicyclic aryl is naphthyl.

Although already included under “heteroaryl”, the term “bicyclic heteroaryl” denotes a 5-10 membered, bicyclic heteroaryl ring which may contain one, two, three or four heteroatoms, selected from among oxygen, sulphur and nitrogen, and contains sufficient conjugated double bonds to form an aromatic system.

Although included by the term “bicyclic cycloalkyls” or “bicyclic aryl”, the term “fused cycloalkyl” or “fused aryl” denotes bicyclic rings wherein the bridge separating the rings denotes a direct single bond. The following are examples of a fused, bicyclic cycloalkyl:

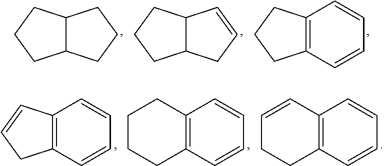

Although included by the term “bicyclic heterocycles” or “bicyclic heteroaryls”, the term “fused bicyclic heterocycles” of “fused bicyclic heteroaryls” denotes bicyclic 5-10 membered heterorings which contain one, two, three or four heteroatoms, selected from among oxygen, sulphur and nitrogen and wherein the bridge separating the rings denotes a direct single bond. The “fused bicyclic heteroaryls” moreover contain sufficient conjugated double bonds to form an aromatic system. Examples include pyrrolizine, indole, indolizine, isoindole, indazole, purine, quinoline, isoquinoline, benzimidazole, benzofuran, benzopyran, benzothiazole, benzothiazole, benzoisothiazole, pyridopyrimidine, pteridine, pyrimidopyrimidine,

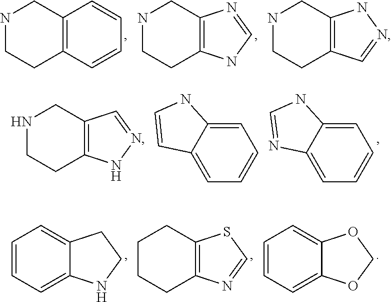

By the term “spiro group” (spiro) are meant 5-10 membered, spirocyclic rings which may optionally contain one, two or three heteroatoms, selected from among oxygen, sulphur and nitrogen, while the ring may be linked to the molecule through a carbon atom or if available through a nitrogen atom. Unless otherwise mentioned, a spirocyclic ring may be provided with an oxo, methyl or ethyl group. Examples of this include:

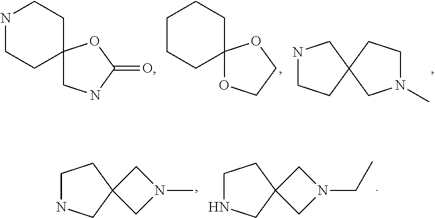

“Halogen” within the scope of the present invention denotes fluorine, chlorine, bromine or iodine. Unless stated to the contrary, fluorine, chlorine and bromine are regarded as preferred halogens.

Compounds of general formula 1 may have acid groups, mainly carboxyl groups, and/or basic groups such as e.g. Amino functions. Compounds of general formula 1 may therefore be present as internal salts, as salts with pharmaceutically usable inorganic acids such as hydrochloric acid, sulphuric acid, phosphoric acid, sulphonic acid or organic acids (such as for example maleic acid, fumaric acid, citric acid, tartaric acid or acetic acid) or as salts with pharmaceutically usable bases such as alkali metal or alkaline earth metal hydroxides or carbonates, zinc or ammonium hydroxides or organic amines such as e.g. diethylamine, triethylamine, triethanolamine, inter alia.

As mentioned previously, the compounds of formula 1 may be converted into the salts thereof, particularly for pharmaceutical use into the physiologically and pharmacologically acceptable salts thereof. These salts may be present on the one hand as physiologically and pharmacologically acceptable acid addition salts of the compounds of formula 1 with inorganic or organic acids. On the other hand, the compound of formula 1 when R is hydrogen may be converted by reaction with inorganic bases into physiologically and pharmacologically acceptable salts with alkali or alkaline earth metal cations as counter-ion. The acid addition salts may be prepared for example using hydrochloric acid, hydrobromic acid, sulphuric acid, phosphoric acid, methanesulphonic acid, acetic acid, fumaric acid, succinic acid, lactic acid, citric acid, tartaric acid or maleic acid. It is also possible to use mixtures of the above-mentioned acids. To prepare the alkali and alkaline earth metal salts of the compound of formula 1 wherein R denotes hydrogen, it is preferable to use the alkali and alkaline earth metal hydroxides and hydrides, of which the hydroxides and hydrides of the alkali metals, particularly sodium and potassium, are preferred, while sodium and potassium hydroxide are particularly preferred.

The compounds of general formula 1 may optionally be converted into the salts thereof, particularly for pharmaceutical use into the pharmacologically acceptable acid addition salts with an inorganic or organic acid. Examples of suitable acids for this purpose include succinic acid, hydrobromic acid, acetic acid, fumaric acid, maleic acid, methanesulphonic acid, lactic acid, phosphoric acid, hydrochloric acid, sulphuric acid, tartaric acid or citric acid. It is also possible to use mixtures of the above-mentioned acids.

The invention relates to the compounds in question, optionally in the form of the individual optical isomers, mixtures of the individual enantiomers or racemates, in the form of the tautomers as well as in the form of the free bases or the corresponding acid addition salts with pharmacologically acceptable acids—such as for example acid addition salts with hydrohalic acids—for example hydrochloric or hydrobromic acid—or organic acids—such as for example oxalic, fumaric, diglycolic or methanesulphonic acid.

The compounds according to the invention may optionally be present as racemates, but may also be obtained as pure enantiomers, i.e. In the (R) or (S) form.

The invention relates to the compounds in question, optionally in the form of the individual optical isomers, diastereomers, mixtures of diastereomers, mixtures of the individual enantiomers or racemates, in the form of the tautomers as well as in the form of the free bases or the corresponding acid addition salts with pharmacologically acceptable acids—such as for example acid addition salts with hydrohalic acids—for example hydrochloric or hydrobromic acid—or organic acids—such as for example oxalic, fumaric, diglycolic or methanesulphonic acid.

The invention relates to the respective compounds of formula 1 in the form of the pharmacologically acceptable salts thereof. These pharmacologically acceptable salts of the compounds of formula 1 may also be present in the form of their respective hydrates (e.g. monohydrates, dihydrates, etc.) as well as in the form of their respective solvates.

By a hydrate of the compound according to the formula 1 is meant, for the purposes of the invention, a crystalline salt of the compound according to formula 1, containing water of crystallisation.

By a solvate of the compound according to formula 1 is meant, for the purposes of the invention, a crystalline salt of the compound according to formula 1, which contains solvent molecules (e.g. ethanol, methanol etc) in the crystal lattice.

The skilled man will be familiar with the standard methods of obtaining hydrates and solvates (e.g. recrystallisation from the corresponding solvent or from water).

### 4. METHODS OF PREPARATION

The compounds 1 claimed may be prepared by known methods (e.g. WO 03/057695). The Examples according to the invention were prepared according to Scheme 1.

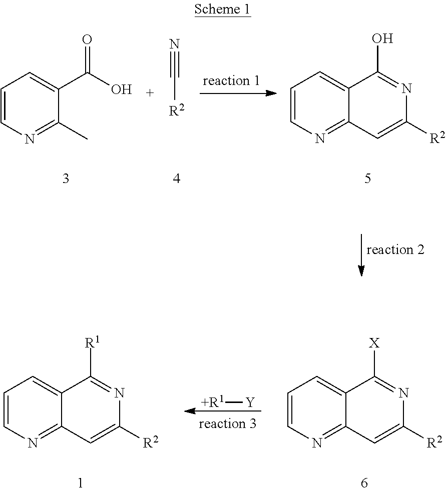

wherein X is a leaving group such as e.g. Cl or triflate,

### Y is —H, —MgBr, —B(OH)₂ and

R¹ and R² are as hereinbefore defined.

Optionally the groups R¹ or R² may subsequently be changed e.g. By reductive amination or amide linking.

### 4.1. Intermediate Products

4.1.1. Synthesis of the Compounds 4 from Scheme 1 (Benzonitrile Derivatives)

### Synthesis of 4-morpholino-3-methoxy-benzonitrile (4.3) (for Examples 10, 70)

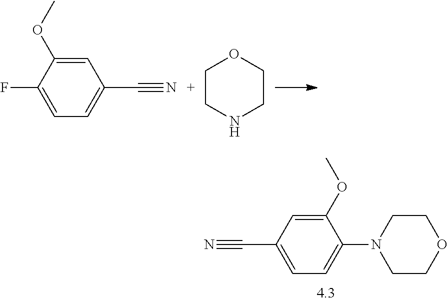

6.7 mL (75 mmol) morpholine was stirred into 50 ml dimethylsulphoxide together with 20 g (141 mmol) potassium carbonate and 10.0 g (66 mmol) 4-fluoro-3-methoxy-benzonitrile for 8 h at 100° C. 500 ml ice water was added to the reaction mixture and the precipitate formed was filtered off and dried.

Yield: 11.2 g (51 mmol=78% of theory)

Analysis: HPLC-MS (method D): Rₜ: 1.36 min, (M+H)⁺: 219

The following were prepared analogously:
3-methyl-4-morpholinobenzonitrile (4.2) see Example 60, 66, 73, 74, 80
3-bromo-4-morpholinobenzonitrile (4.4) see Example 145
4-morpholinobenzonitrile (4.1) is commercially obtainable.

### 4.1.2. Synthesis of R¹ Derivatives (Amine Derivatives)

### Synthesis of N-(4-aminocyclohexyl)-2,2,2-trifluoro-N-methyl-acetamide (for Example 9)

### Step 1

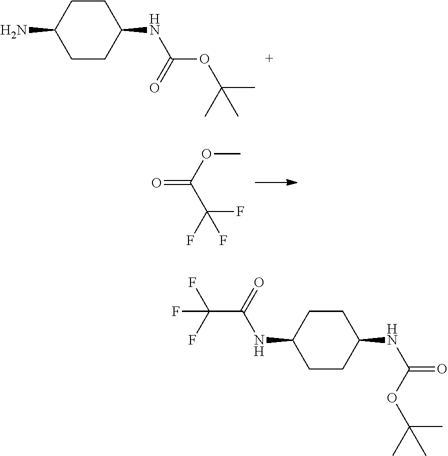

22.1 g (103 mmol) tert-butyl cis-(4-aminocyclohexyl)-carbamate and 11 ml (110 mmol) methyl trifluoroacetate were stirred into 110 ml of methanol 4 h at ambient temperature, the reaction mixture was cooled in the ice bath, the precipitate formed was suction filtered and washed with diethyl ether.

Yield: 17.6 g (57 mmol=55% of theory)

### Step 2

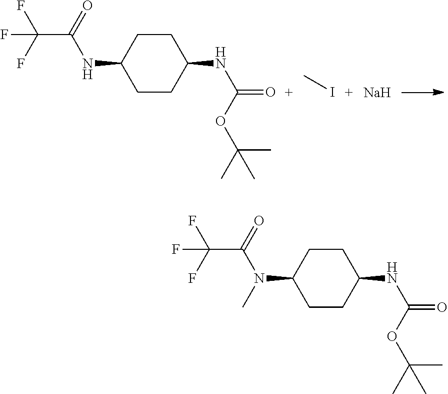

The reaction was carried out under a nitrogen atmosphere.

8.30 g (26.8 mmol) tert-butyl cis-[4-(2,2,2-trifluoroacetylamino)-cyclohexyl]-carbamate were placed in 100 ml N,N-dimethylacetamide and 1.28 g (32 mmol) sodium hydride (60%) were added. After 20 min stirring at ambient temperature 4.54 g (32 mmol) methyl iodide was added, the reaction mixture was stirred further overnight at ambient temperature. The mixture was poured onto 800 ml ice water, the precipitate was suction filtered and washed with water and petroleum ether. Then it was recrystallised from 200 ml diisopropylether and 10 ml acetonitrile.

Yield: 11.0 g (34 mmol)

### Step 3

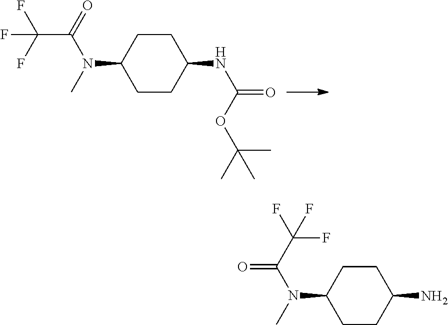

4.20 g (13 mmol) tert-butyl cis-{-[4-methyl-(2,2,2-trifluoracetyl)-amino]-cyclohexyl}-carbamate was stirred overnight at ambient temperature with 30 ml trifluoroacetic acid in 60 ml dichloromethane. It was evaporated down and the residue was triturated with diethyl ether, and the precipitate was filtered off.

Yield: 5.30 g (16 mmol=121% of theory)

### Synthesis of 4-(3-diethylaminopropoxy)-phenylamine (for Example 20)

### Step 1:

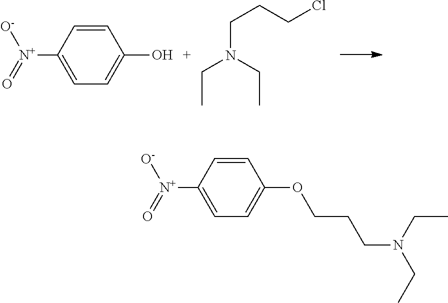

25.0 g (0.18 mol) p-nitrophenol, 32.3 g (0.22 mol) diethylaminopropyl chloride and 29.9 g (0.22 mol) potassium carbonate were refluxed in 300 ml dimethylformamide overnight. The solvent was eliminated from the reaction mixture, the residue was taken up in ethyl acetate, the organic phase was washed with water and sodium hydroxide solution (2 mol/l), dried, filtered and the solvent was removed from the filtrate.

Yield: 28.9 g (15.7 mmol=64% of theory)

### Step 2:

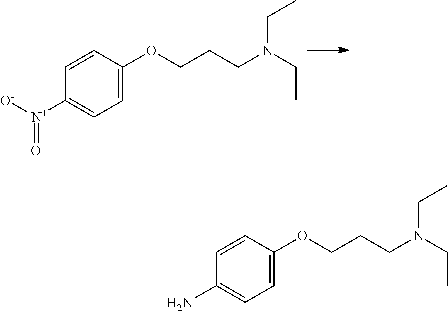

29.0 g (0.12 mol) diethyl-[3-(4-nitrophenoxy)-propyl]-amine and 2.9 g Pd/C were hydrogenated in 300 ml of ethanol at ambient temperature. The catalyst was filtered off and the solvent removed.

Yield: 39.0 g (0.18 mol)

### Synthesis of R-3-(aminomethyl)-1-methyl-pyrrolidine (for Example 64)

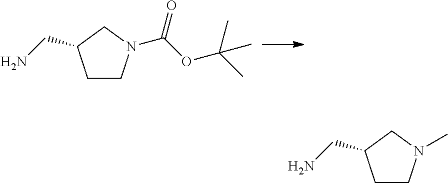

6.00 ml (6 mmol) lithium aluminium hydride was placed in tetrahydrofuran, then 0.30 g (1.49 mmol) R-3-(aminomethyl)-1-N-tert-butyloxycarbonyl-pyrrolidine dissolved in 6 ml of tetrahydrofuran was added dropwise at ambient temperature. The reaction mixture was stirred overnight at ambient temperature, then cooled and 1.5 ml of water, 10 ml THF and 1.5 ml 4N sodium hydroxide solution were added with stirring and the mixture was stirred for 10 min. The suspension was filtered through kieselguhr, washed with tetrahydrofuran and the solvent was eliminated from the filtrate.

Yield: 130 mg (1.14 mmol=76% of theory)

Analysis: ESI-MS, (M+H)⁺: 115

### 1-(3-aminopropyl)tetrahydropyrimidin-2-one (for Example 23)

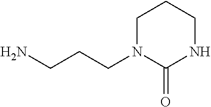

1-(3-aminopropyl)tetrahydropyrimidin-2-one may be synthesised according to the following literature: Tang, Peng Cho; Miller, Todd; Li, Xiaoyuan; Sun, Li; Wei, Chung Chen; Shirazian, Shahrzad; Liang, Congxin; Vojkovsky, Tomas; Nematalla, Asaad S. WO2001060814

### N-methyl-N′-piperazine urea (for Example 123)

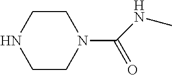

N-methyl-W-piperazine urea may be synthesised according to the following literature
Zhao, Matthew; Yin, Jingjun; Huffman, Mark A.; McNamara, James M. Tetrahedron (2006), 62(6), 1110-1115.

### 4,5,7,8-tetrahydro-1H-imidazo[4,5-d]azepine (for Example 133)

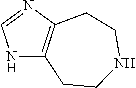

4,5,7,8-tetrahydro-1H-imidazo[4,5-d]azepine may be synthesised according to the following literature: Dorwald, Florencio Zaragoza; Andersen, Knud Erik; Jorgensen, Tine Krogh; Peschke, Bernd; Wulff, Birgitte Schjellerup; Pettersson, Ingrid; Rudolf, Klaus; Stenkamp, Dirk; Hurnaus, Rudolf; Muller, Stephan Georg; Krist, Bernd WO2000063208

### 2-methyl-N-1-2-pyrimidinyl-1,2-propanediamine (for Example 139)

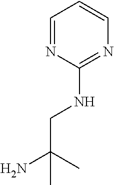

2-methyl-N-1-2-pyrimidinyl-1,2-propanediamine may be synthesised according to the following literature: Matsuno, Kenji; Ueno, Kimihisa; Iwata, Yasuhiro; Matsumoto, Yuichi; Nakanishi, Satoshi; Takasaki, Kotaro; Kusaka, Hideaki; Nomoto, Yuji; Ogawa, Akira WO2002051836

### 4.1.3. Synthesis of R¹ Derivatives (Alcohol Derivatives)

### Synthesis of (S)-4-(hydroxymethyl)-1-((S)-1-phenylethyl)pyrrolidin-2-one (for Example 85)

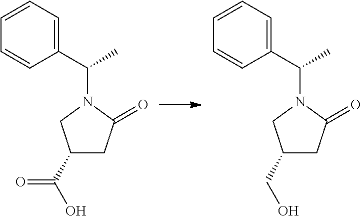

500 mg (2.14 mmol) (1′S,3S)-1-(1″phenylethyl)-5-oxo-3-pyrrolidine carboxylic acid was dissolved in 5 mL THF, then the solution was cooled to 5° C. 1.83 mL (3.6 mmol) BH₃*SMe₂ (2 mol/l in THF) was slowly added dropwise and the reaction solution was slowly heated to 25° C. and stirred for another 5 h at 25° C. The reaction mixture was combined with 2.5 mL saturated NaHCO₃ solution and after the foaming had stopped the mixture was extracted with 2× dichloromethane, the organic phase was washed with saturated NaCl solution, dried on MgSO₄ and evaporated down.

Yield: 500 mg (2.05 mmol=96% d. Th)

Analysis: HPLC-MS (method D): Rₜ=1.21 min (M+H)⁺=220

(R)-4-(hydroxymethyl)-1-((R)-1-phenylethyl)pyrrolidin-2-one was prepared analogously (Example 84).

### 4.2. Reaction 1 of Scheme 1: Synthesis of Compounds of Formula 5

### Synthesis of 7-(4-morpholin-4-yl-phenyl)-[1,6]naphthyridin-5-ol (5.1)

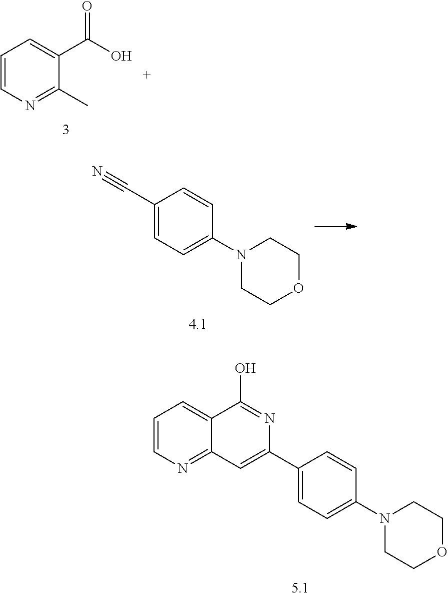

The reaction was carried out under an argon atmosphere.

4.05 g (29.5 mmol) 2-methyl-nicotinic acid was suspended in 130 ml of tetrahydrofuran, and cooled to −65° C. with a bath of ethanol/dry ice. 43.5 ml (65 mmol) lithium diisopropylamide (1.5 mol/l in tetrahydrofuran) was added dropwise within 30 minutes and the mixture was stirred for 2.5 h in the ice bath (0° C.). Then it was cooled again to −65° C. and a solution of 6.12 g (32.5 mmol) 4-morpholine-benzonitrile in 70 ml of tetrahydrofuran was added dropwise within 30 minutes. Then the reaction mixture was stirred overnight at ambient temperature. The suspension was combined with 200 ml of water and the solvent was distilled off.

The aqueous residue was combined with 200 ml ethyl acetate and stirred for 2 h, then the precipitate was suction filtered and dried.

Yield: 3.75 g (12 mmol=41% of theoretical)

Analysis: ESI-MS: (M+H)⁺: 308

The following compounds were prepared analogously to the methods described (see Table 1).

TABLE 1
Further [1,6]-naphthyridin-5-ol derivatives 5.2-5.6

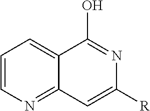

| product | | HPLC-MS, | | HPLC-MS |
|---|---|---|---|---|
| number | R | Rₜ(min) | (M + H)⁺ | method |
| 5.2 see Examples 60, 66, 73, 74, 80 | 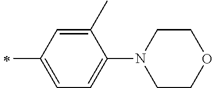 | 1.17 | 322 | method D |
| 5.3 see Examples 10, 70 | 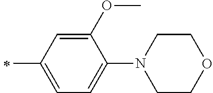 | 1.07 | 338 | method D |
| 5.4 see Example 145 | 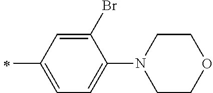 | 1.23 | 387 | method D |
| 5.5 see Examples 87-93, 116- 124, 128 | 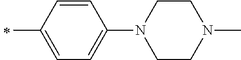 | 0.97 | 321 | method D |
| 5.6 see Example 68 | 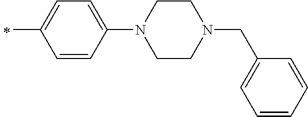 | 1.10 | 397 | method D |

### 4.3. Reaction 2 of Scheme 1: Synthesis of Compounds of Formula 6

4.3.1. Synthesis of Compounds of Formula 6 (5-chloro-[1,6]naphthyridine Derivatives)

### Synthesis of 5-chloro-7-(4-morpholinophenyl)-[1,6]naphthyridine (6.1)

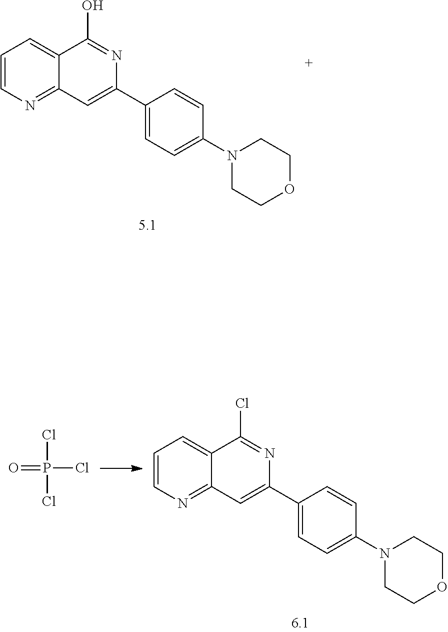

5.0 g (16 mmol) 7-(4-morpholino-phenyl)[1,6]-naphthyridin-5-ol (5.1) and 0.50 ml (2.3 mmol) N,N-diethylaniline were stirred into 100 ml (1090 mmol) phosphorus oxychloride overnight at 120° C.

The reaction mixture was evaporated down, the residue was combined with approx. 100 ml of water, made neutral with Na₂CO₃ solution, and extracted with methylene chloride. The organic phase was dried and evaporated down.

Yield: 5.3 g (13 mmol=80% of theoretical)

Analysis (method D): Rₜ: 1.57 min, (M+H)⁺: 326/328 (Cl)

The following compounds were prepared analogously to the method described above (see Table 2).

TABLE 2
Further 5-chloro-[1,6]naphthyridine derivatives 6.2-6.5

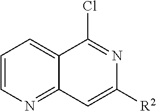

| product | | HPLC-MS, | | HPLC-MS |
|---|---|---|---|---|
| number | R² | Rₜ(min) | (M + H)⁺ | method |
| 6.2 see Examples 60, 66, 73, 74, 80 | 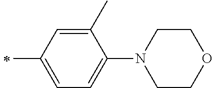 | 1.71 | 340/342 | method D |
| 6.3 see Examples 10, 70 | 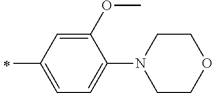 | 1.38 | 356/358 | method D |
| 6.4 see Example 145 | 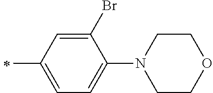 | 1.86 | 406 | method D |
| 6.5 see Examples 87-93, 116- 124, 128 | 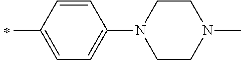 | 1.26 | 339/341 | method D |

4.3.2. Synthesis of Compounds of Formula 6 ([1,6]naphthyridin-5-yl-trifluoromethanesulphonic Acid Ester Derivatives)

### Synthesis of 7-(4-morpholin-4-yl-phenyl)-[1,6]naphthyridin-5-yl-trifluoromethanesulphonic acid ester (6.6)

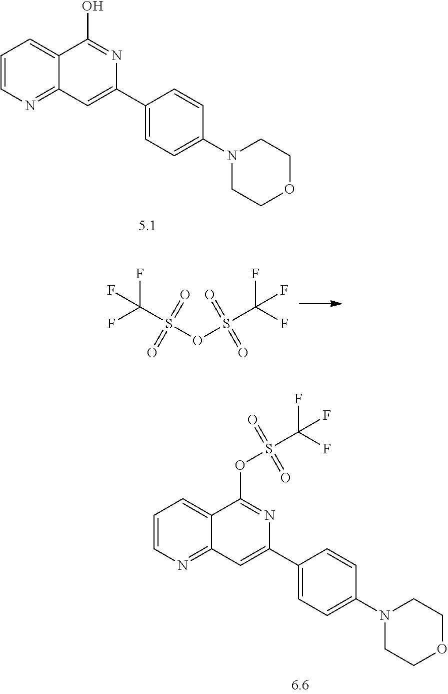

12.3 g (40 mmol) 5.1 were placed in 800 ml dichloromethane, then 3.16 ml (40 mmol) pyridine were added. At 0° C. a solution of 7.26 ml (44 mmol) trifluoromethanesulphonic acid anhydride in dichloromethane was added dropwise and after the addition the reaction mixture was heated to ambient temperature. Then a further 7.26 ml (44 mmol) trifluoromethanesulphonic acid anhydride were added at ambient temperature and the mixture was stirred for another 1 h. The reaction mixture was mixed with water and extracted with dichloromethane. The organic phase was dried with MgSO₄, filtered and the solvent was removed from the filtrate.

The residue was purified by chromatography (silica gel, cyclohexane/ethyl acetate: 70/30 to 50/50) and corresponding fractions were evaporated down.

Yield: 9.70 g (22.1 mmol=55% of theory)

The following compound was prepared analogously to the method described above (see Table 3).

TABLE 3
A further trifluoromethanesulphonic acid ester

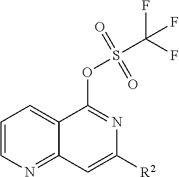

| Product | | HPLC-MS, | | HPLC-MS |
|---|---|---|---|---|
| No. | R² | Rₜ(min), | (M + H)⁺ | method |
| 6.7 see Example 68 | 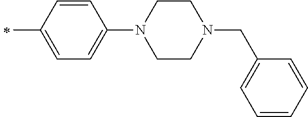 | 1.56 | 529 | method D |

### 4.4. Reaction 3 of Scheme 1 (Synthesis of the Patent Examples of Formula 1)

### EXAMPLE 1

### 5-[(1H-indazol-6-yl)amino]-7-(4-morpholinophenyl)-[1,6]naphthyridine

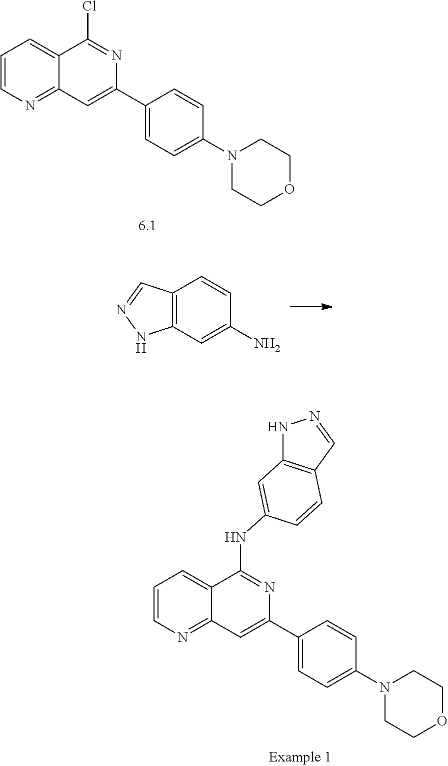

150 mg (0.41 mmol) 6.1 and 300 mg (2.25 mmol) 6-aminoindazole were stirred for 2 h at 100° C. Then 0.5 ml N-methylpyrrolidone and 0.10 ml (0.41 mmol) dioxanic hydrochloric acid (4 mol/l) were added and the mixture was stirred for 4 h at 100° C. The reaction mixture was combined with dichloromethane and a little methanol, the precipitate formed was filtered off, then stirred with methanol, suction filtered and dried.

Yield: 130 mg (0.31 mmol=74% of theory)

Analysis: HPLC-MS (method A): Rₜ: 2.51 min

Example 106 was obtained analogously.

### EXAMPLE 2

### 2-[7-(4-morpholin-4-yl-phenyl)-[1,6]naphthyridin-5-ylamino]-nicotinamide

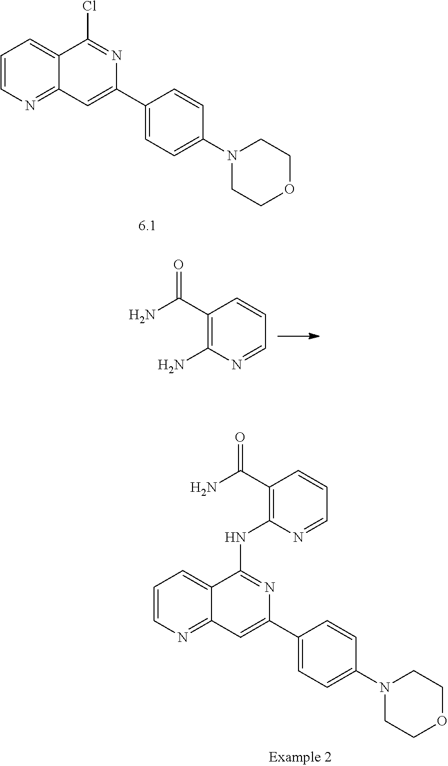

100 mg (0.31 mmol) of 6.1, 50 mg (0.37 mmol) 2-aminonicotinamide, 37 mg (0.06 mmol) 2,2′-bis-(diphenylphosphino)-1,1′-binaphthyl, 24.2 mg (0.03 mmol) of tris(dibenzylideneacetone)-dipalladium(0) and 0.41 g (1.25 mmol) of caesium carbonate were refluxed in 2 ml of toluene for 5 h with stirring. The reaction mixture was dissolved in dichloromethane/methanol and filtered through kieselguhr. The filtrate was evaporated down, the residue was dissolved in dichloromethane/water, the phases were separated. The organic phase was washed 2× with water, dried on MgSO₄, filtered and evaporated down. The residue was purified by chromatography (silica gel, dichloromethane 100% to dichloromethane:methanol 99:1). The corresponding fractions were evaporated down. The mixture was again purified by chromatography (RP-HPLC), the acetonitrile was distilled off from the corresponding fractions, the aqueous solution was made basic with K₂CO₃ and the precipitate was suction filtered.

Yield: 15 mg (0.04 mmol=11% of theory)

Analysis: HPLC-MS (method A): Rₜ: 2.40 min

### EXAMPLE 3

### N-methyl-(4-morpholinophenyl)-[1,6]naphthyridin-5-yl-amine

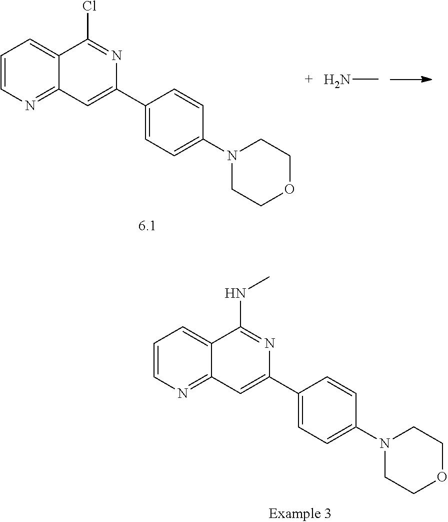

200 mg (0.61 mmol) 6.1, 1.75 mL (3.5 mmol) methylamine solution (2 mol/l in THF) were stirred into 0.8 mL N-methylpyrrolidone for 5 h at 120° C. in a pressurised test tube. The mixture was evaporated down and combined with acetonitrile/water and trifluoroacetic acid and purified by chromatography (RP-HPLC). The corresponding fractions were freeze-dried.

Yield: 210 mg (0.48 mmol=78% of theory)

Analysis: HPLC-MS (method C): Rₜ: 1.08 min, (M+H)⁺: 321

Examples 7, 60, 74, 80, 87-91, 94, 96, 97, 100, 101, 108, 109, 117-127, and 129-131 were obtained analogously to Example 3.

### EXAMPLE 4

### 4-[7-(4-morpholinophenyl)-[1,6]naphthyridin-5-yloxy]-butan-1-ol

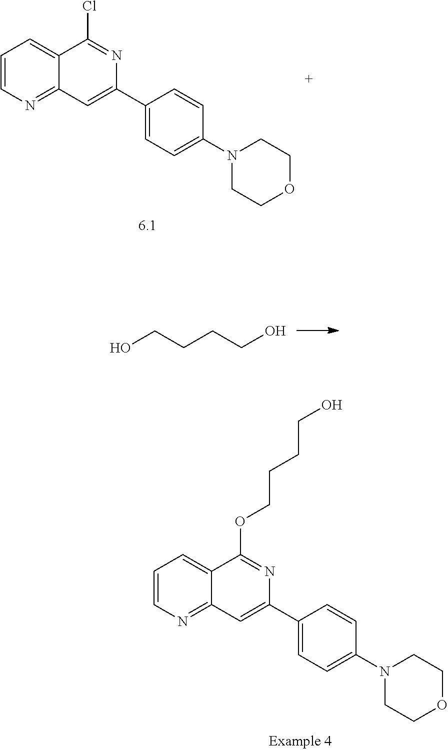

136.4 μl (1.54 mmol) 1,4-butanediol was placed in 1.5 ml dimethylacetamide and 43 mg (1.08 mmol) sodium hydride (60%) were added and the mixture was stirred for 15 min at ambient temperature. Then 100 mg (0.31 mmol) 6.1 was added and the mixture was stirred for 2 h at 70° C. The reaction mixture was added to water and the precipitate was suction filtered. The residue was purified by chromatography (RP-HPLC-MS). The corresponding fractions were freeze-dried.

Yield: 100 mg (0.20 mmol=66% of theory)

Analysis: HPLC-MS (method C): Rₜ: 1.13 min, (M+H)⁺: 380

The following Examples were prepared analogously to Example 4: Examples 5, 6, 72, 73, 83, 116, 133.

### EXAMPLE 8

### 2-{3-[7-(4-morpholin-4-yl-phenyl)-[1,6]naphthyridin-5-ylamino]-propylamino}-acetamide

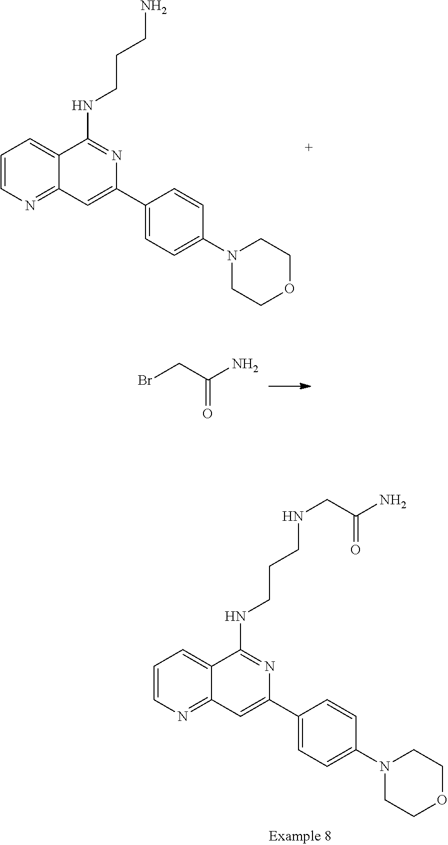

N*1*-[7-(4-morpholin-4-yl-phenyl)-[1,6]naphthyridin-5-yl]propane-1,3-diamine was prepared analogously to Example 3 using educt 6.1.

50 mg (0.11 mmol) N*1*-[7-(4-morpholin-4-yl-phenyl)-[1,6]naphthyridin-5-yl]-propane-1,3-diamine was dissolved in 1 ml dimethylformamide and 40 mg potassium carbonate were added. Then the reaction mixture was cooled in the ice bath, then 16 mg (0.12 mmol) 2-bromoacetamide dissolved in 1 ml dimethylfomamide was added dropwise within 5 min. The reaction mixture was heated to ambient temperature and filtered. The filtrate was purified by chromatography (RP-HPLC-MS), the corresponding fractions were freeze-dried.

Yield: 8 mg (0.02 mmol=18% of theory)

Analysis: HPLC-MS (method D): Rₜ: 1.10 min, (M+H)⁺: 421

### EXAMPLE 9

### N-methyl-N′-[7-(4-morpholin-4-yl-phenyl)-[1,6]naphthyridin-5-yl]-cyclohexane-1,4-diamine

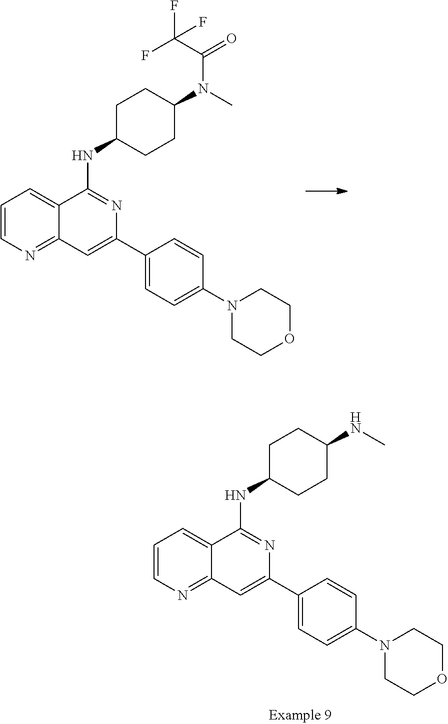

2,2,2-trifluoro-N-methyl-N-{4-[7-(4-morpholin-4-yl-phenyl)-[1,6]naphthyridin-5-ylamino]-cyclohexyl}-acetamide was prepared analogously to Example 3 using educt 6.1.

120 mg (0.19 mmol) 2,2,2-trifluoro-N-methyl-N-{4-[7-(4-morpholin-4-yl-phenyl)-[1,6]naphthyridin-5-ylamino]-cyclohexyl}-acetamide was suspended in 2 ml of methanol and combined with 200 μl sodium hydroxide solution (2 mol/l). The reaction mixture was stirred overnight at ambient temperature. Another 400 μl sodium hydroxide solution (2 mol/l) were added. The mixture was evaporated down, the residue was purified by chromatography (RP-HPLC-MS), the corresponding fractions were freeze-dried.

Yield: 85 mg (0.16 mmol=84% of theory)

Analysis: HPLC-MS (method D): Rₜ: 1.16 min, (M+H)⁺: 418

### EXAMPLE 10

### 5-[(pyrrolidin-3-yl-methylamino]-7-(4-morpholino-3-methoxyphenyl)-[1,6]naphthyridine

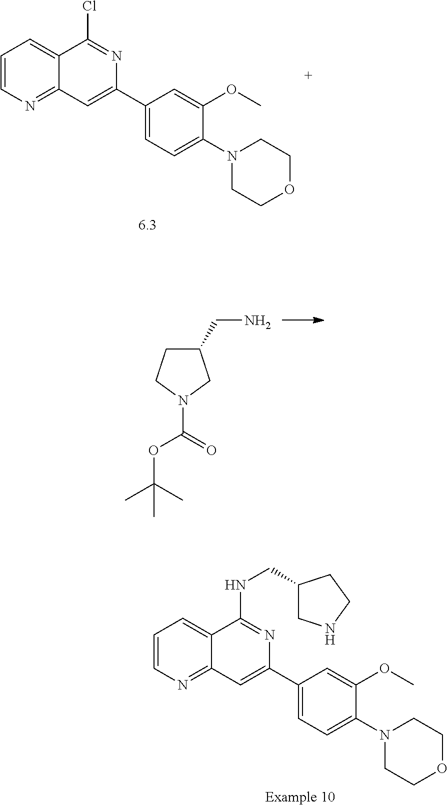

100 mg (0.37 mmol) 6.3 was stirred with 170 mg (1.04 mmol) (R)-3-aminomethyl-1-N-tert-butyloxycarbonyl-pyrrolidine for 25 min at 225° C. in 0.5 mL N-methylpyrrolidine. The mixture was diluted with acetonitrile/water and purified by chromatography (RP-HPLC-MS). The corresponding fractions were freeze-dried.

Yield: 40 mg (0.086 mmol=31% of theory)

Analysis: HPLC-MS (method D): Rₜ: 1.07 min, (M+H)⁺: 420

The following compounds were obtained analogously to Example 10: Examples 61, 70, 71, 102-104, 111, 112, 128.

### EXAMPLE 62

### 4-[7-(4-morpholin-4-yl-phenyl)-[1,6]naphthyridin-5-ylamino]-cyclohexanol

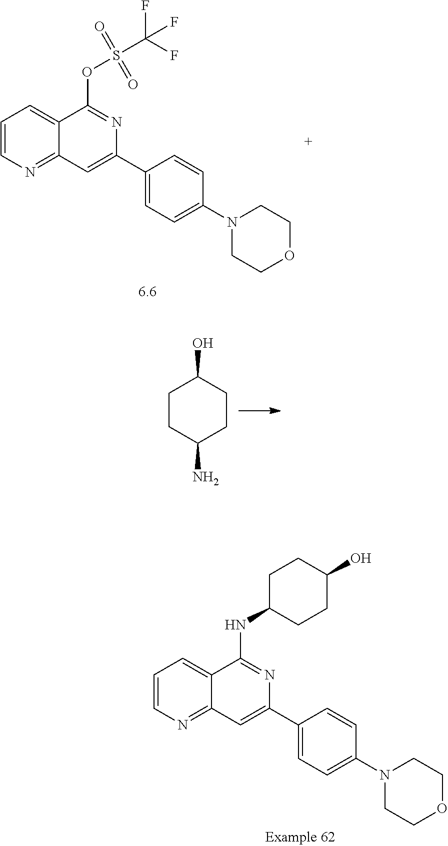

50 mg (0.11 mmol) 6.6, 38 mg (0.25 mmol) cis-4-aminocyclohexanol hydrochloride and 50 μl (0.29 mmol) diisopropylethylamine were stirred into 0.5 ml N-methylpyrrolidone for 6 h at 80° C.

The mixture was purified by chromatography (RP-HPLC-MS). The corresponding fractions were freeze-dried.

Yield: 35 mg (0.07 mmol=59% of theory)

Analysis: HPLC-MS (method D): Rₜ: 1.24 min, (M+H)⁺: 405

Examples 11-59, 64, 75, 76, 114, 115, 134-139, 141, 143 and 144 were obtained analogously to Example 62.

### EXAMPLE 63

### Morpholin-2-yl-methyl-[7-(4-morpholin-4-yl-phenyl)-[1,6]naphthyridin-5-yl]-amine

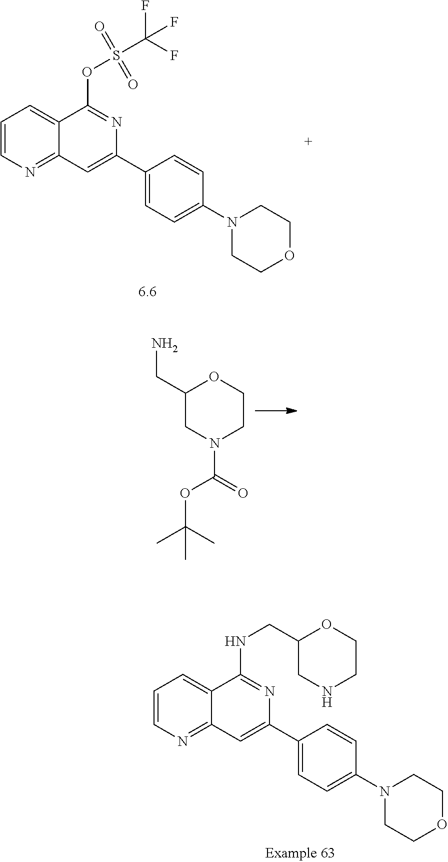

70 mg (0.16 mmol) 6.6 and 76 mg (0.35 mmol) 2-aminomethyl-4-tert-butyloxycarbonyl-morpholine were stirred into 0.5 ml N-methylpyrrolidone for 2 h at 80° C. The reaction mixture was combined with 1 ml trifluoroacetic acid and stirred overnight at ambient temperature. The mixture was purified by chromatography (RP-HPLC-MS). The corresponding fractions were freeze-dried.

Yield: 65 mg (0.13 mmol=79% of theory)

Analysis: HPLC-MS (method D): Rₜ: 1.11 min, (M+H)⁺: 406

The following Examples were prepared analogously to Example 63: Examples 67, 68, 77, 78, 86.

### EXAMPLE 65

### 4-[7-(4-morpholin-4-yl-phenyl)-[1,6]naphthyridin-5-yloxymethyl]-pyrrolidin-2-one

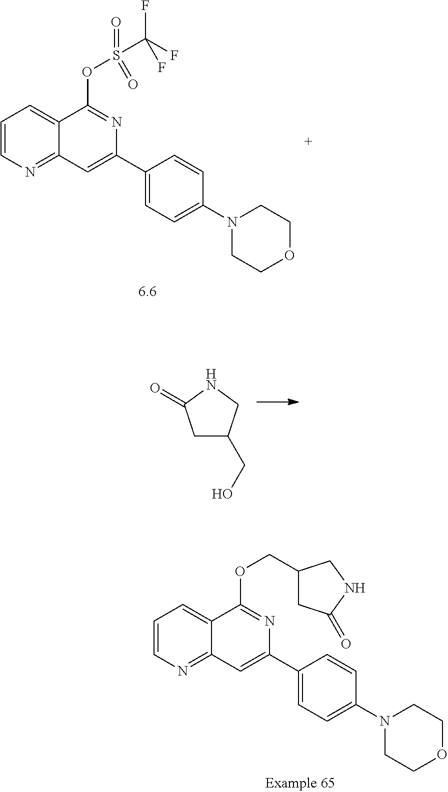

32.8 mg (0.28 mmol) 4-hydroxymethylpyrrolidin-2-one was placed in 1 ml dimethylacetamide and 11.5 mg (0.29 mmol) sodium hydride (60%) were added and the mixture was stirred for 15 min at ambient temperature. Then 50 mg (0.11 mmol) 6.6 was added and the mixture was stirred for 2 h at 70° C. The reaction mixture was purified by chromatography (RP-HPLC-MS). The corresponding fractions were freeze-dried.

Yield: 15 mg (0.03 mmol=25% of theory)

Analysis: HPLC-MS (method D): Rₜ: 1.18 min, (M+H)⁺: 405

### EXAMPLE 66

### (S)-4-(2-methyl-4-(5-(piperidine-3-ylmethoxy)-[1,6]naphthyridin-7-yl)phenyl)morpholine

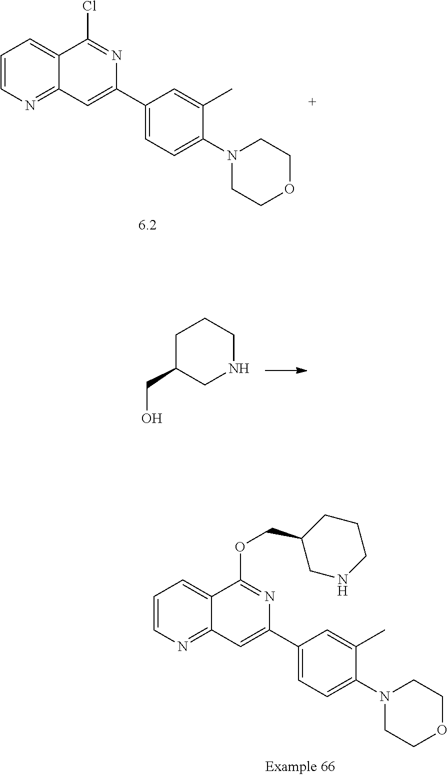

111 mg (0.52 mmol) (S)-1-tert-butyloxycarbonyl-3-(hydroxymethyl)-piperidine and 21 mg (0.52 mmol) sodium hydride (60%) were placed in 0.5 ml dimethylacetamide and stirred for 15 min at ambient temperature. Then 70 mg (0.22 mmol) 6.2 was added and the mixture was stirred for 2 h at 70° C. 0.5 mL trifluoroacetic acid were added and the mixture was stirred for 4 h at 40° C. and overnight at 25° C. The reaction mixture was purified by chromatography (RP-HPLC-MS). The corresponding fractions were freeze-dried.

Yield: 70 mg (0.13 mmol=64% of theory)

Analysis: HPLC-MS (method D): Rₜ: 1.64 min, (M+H)⁺: 519

The following compounds were obtained analogously to Example 66: Example 69, 81, 110, 145.

### EXAMPLE 79

### 5-Ethoxy-7-(4-morpholin-4-yl-phenyl)-[1,6]naphthyridine

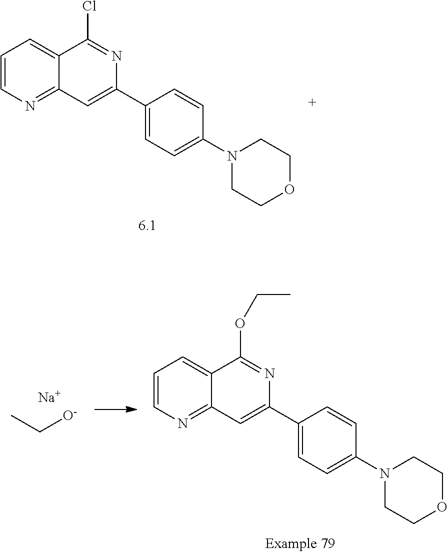

100 mg (0.31 mmol) 6.1 was placed in 0.5 ml N-methyl-pyrrolidone and then 100 mg (1.44 mmol) sodium methoxide were added. The reaction mixture was stirred for 1 h at 50° C.

The mixture was purified by chromatography (RP-HPLC-MS), the corresponding fractions were freeze-dried.

Yield: 72 mg (0.22 mmol=70% of theory)

Analysis: HPLC-MS (method D): Rₜ: 1.40 min, (M+H)⁺: 336

Example 95 was prepared analogously.

### EXAMPLE 82

### 3-[7-(4-morpholin-4-yl-phenyl)-[1,6]naphthyridin-5-yl]-prop-2-yn-1-ol

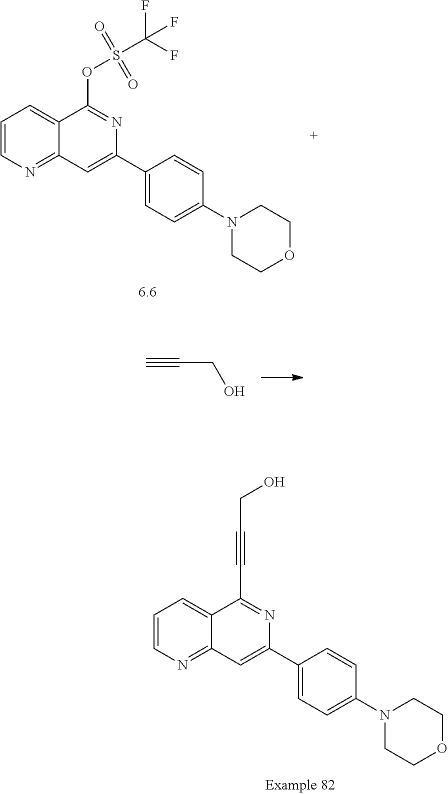

The reaction was carried out under an argon atmosphere.

250 mg (0.57 mmol) 6.6, 95.7 mg (1.71 mmol) propargylalcohol, 300 μl (1.75 mmol) diisopropylethylamine, 41 mg (0.06 mmol) triphenylphosphine palladium(II)chloride and 5.5 mg (0.03 mmol) copper(I) iodide were stirred into 2 ml of dry acetonitrile for 2 h at 80° C. The reaction mixture was diluted with dichloromethane/methanol and filtered through kieselguhr, the filtrate was evaporated down. The residue was dissolved in dichloromethane and extracted with aqueous ammonia and saturated sodium chloride solution, the org. Phase was evaporated down and purified by chromatography. (Silica gel, dichloromethane 100 to dichloromethane/methanol: 95/5). The corresponding fractions were evaporated down.

Yield: 150 mg (0.43 mmol=76% of theory)

Analysis: HPLC-MS (method D): Rₜ: 1.26 min, (M+H)⁺: 346

### EXAMPLE 84

### (R)-4-[7-(4-morpholin-4-yl-phenyl)-[1,6]naphthyridin-5-yloxymethyl]-pyrrolidin-2-one

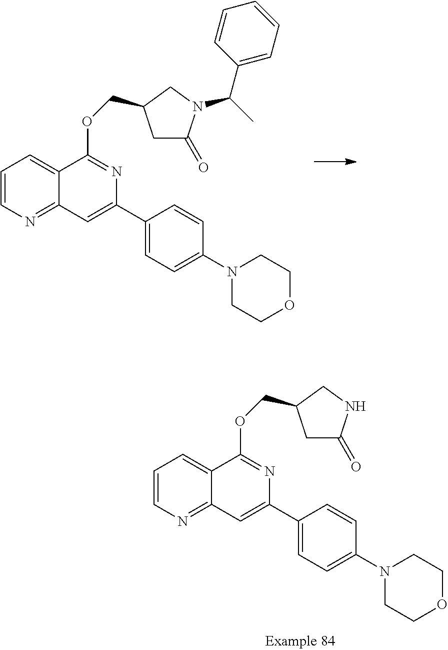

(R,R)-4-[7-(4-morpholin-4-yl-phenyl)-[1,6]naphthyridin-5-yloxymethyl]-1-(1-phenyl-ethyl)-pyrrolidin-2-one was prepared analogously to Example 10 using educt 6.1. 30 mg (0.06 mmol) (R,R)-4-[7-(4-morpholin-4-yl-phenyl)-[1,6]naphthyridin-5-yloxymethyl]-1-(1-phenyl-ethyl)-pyrrolidin-2-one was dissolved in 1 ml trifluoroacetic acid and heated for 45 min at 150° C. in the microwave with stirring. The mixture was purified by chromatography (RP-HPLC-MS), the corresponding fractions were freeze-dried.

Yield: 20 mg (0.05 mmol=84% of theory)

Analysis: HPLC-MS (method D): Rₜ: 1.20 min, (M+H)⁺: 405

Example 85 was prepared analogously.

### EXAMPLE 92

### 1-ethyl-3-(1-{7-[4-(4-methyl-piperazin-1-yl)-phenyl]-[1,6]naphthyridin-5-yl}-azetidin-3-yl)-urea

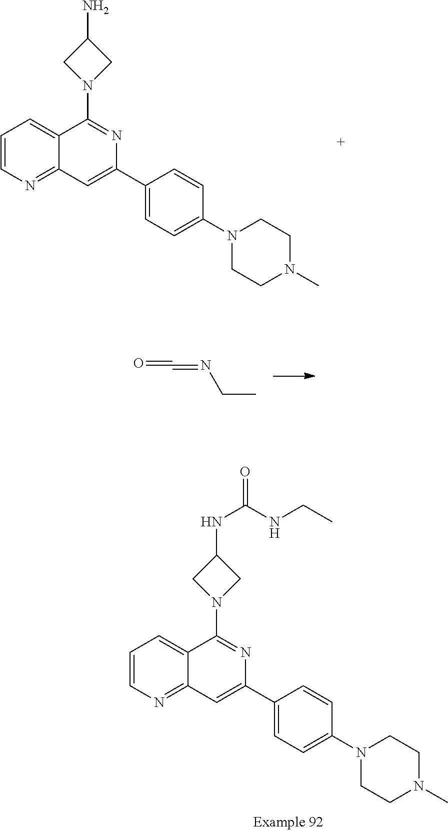

1-{7-[4-(4-methyl-piperazin-1-yl)-phenyl]-[1,6]naphthyridin-5-yl}-azetidin-3-yl-amine was prepared according to Example 63 using educt 6.5 at a reaction temperature of 110° C. and 24 h reaction time.

77.1 mg (0.21 mmol) 1-{7-[4-(4-methyl-piperazin-1-yl)-phenyl]-[1,6]naphthyridin-5-yl}-azetidin-3-yl-amine was placed in 2 ml dry dichloromethane and 0.5 ml dry dimethylformamide, then 211 μl (1.24 mmol) diisopropylethylamine was added. The mixture was cooled to 0° C. and 14.6 mg (0.21 mmol) ethyl isocyanate, dissolved in dichloromethane, were slowly added dropwise. The reaction mixture was stirred for 30 min at ambient temperature, then evaporated down. The residue was purified by chromatography (RP-HPLC).

Yield: 55 mg (0.12 mmol=60% of theory)

Analysis: HPLC-MS (method L): Rₜ: 1.51 min, (M+H)⁺: 446

### EXAMPLE 93

### 2-({7-[4-(4-methyl-piperazin-1-yl)-phenyl]-[1,6]naphthyridin-5-ylamino}-methyl)-pyrrolidin-1-carboxylic acid ethylamide

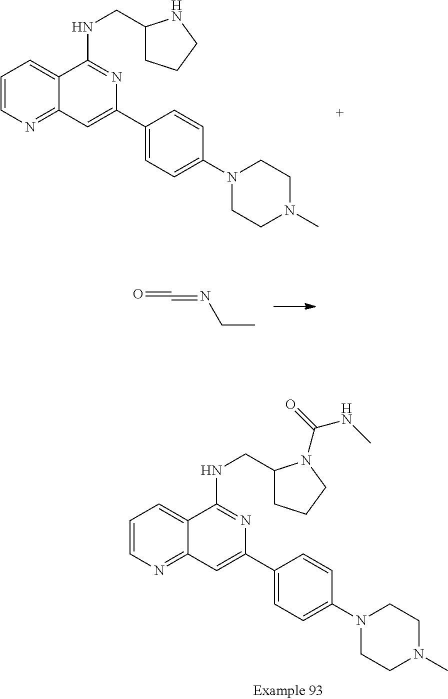

{7-[4-(4-methyl-piperazin-1-yl)-phenyl]-[1,6]naphthyridin-5-yl}-pyrrolidin-2-yl-methylamine was prepared analogously to Example 62 using educt 6.5 at a reaction temperature of 110° C. and 24 h reaction time.

39 mg (0.09 mmol) {7-[4-(4-methyl-piperazin-1-yl)-phenyl]-[1,6]naphthyridin-5-yl}-pyrrolidin-2-yl-methylamin was placed in 2 ml dry dichloromethane, then 45 μl (0.28 mmol) diisopropylethylamine was added. The mixture was cooled to 0° C. and 8.2 mg (0.12 mmol) ethyl isocyanate, dissolved in dichloromethane, was slowly added dropwise. The reaction mixture was stirred for 30 min at ambient temperature, then evaporated down. The residue was purified by chromatography (silica gel, dichloromethane:methanol:ammonia=9:1:0.1).

Yield: 23 mg (0.05 mmol=50% of theory)

Analysis: HPLC-MS (method F): Rₜ: 1.59 min, (M+H)⁺: 474

### EXAMPLE 98

### 4-[7-(4-morpholin-4-yl-phenyl)-[1,6]naphthyridin-5-yl]-piperazine-1-carboxylic acid amide

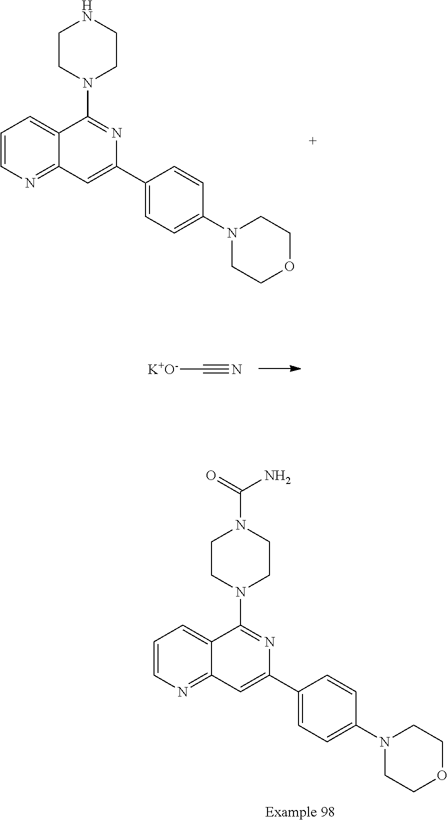

7-(4-morpholin-4-yl-phenyl)-5-piperazin-1-yl-[1,6]naphthyridine was prepared analogously to Example 3 using educt 6.1.

63 mg (0.17 mmol) 7-(4-morpholin-4-yl-phenyl)-5-piperazin-1-yl-[1,6]naphthyridine was placed in 6 ml of ethanol and 50 μl (0.84 mmol) glacial acetic acid were added, then 14.3 mg (0.18 mmol) potassium cyanate were added. The reaction mixture was left overnight at ambient temperature with stirring, then diluted with methanol. It was purified by chromatography (RP-HPLC, basic, % ACN 15->60 in 10 min), and the corresponding fractions were freeze-dried.

Yield: 45 mg (0.11 mmol=64% of theory)

Analysis: HPLC-MS (method I): Rₜ: 1.53 min, (M+H)⁺: 419

Examples 99 and 105 were obtained analogously.

### EXAMPLE 107

### 1-[7-(4-morpholin-4-yl-phenyl)-[1,6]naphthyridin-5-yl]-pyrrolidine-3-carboxylic acid amide

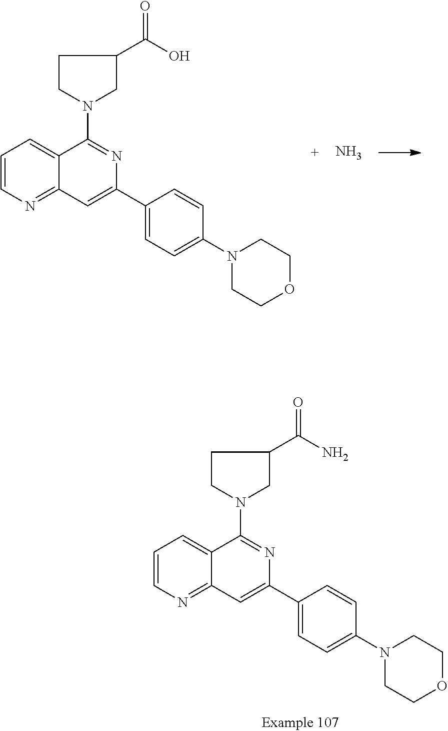

1-[7-(4-morpholin-4-yl-phenyl)-[1,6]naphthyridin-5-yl]-pyrrolidine-3-carboxylic acid was obtained analogously to Example 3 using educt 6.1.

40 mg (0.10 mmol) 1-[7-(4-morpholin-4-yl-phenyl)-[1,6]naphthyridin-5-yl]-pyrrolidine-3-carboxylic acid, 38 mg (0.12 mmol) 2-(1H-benzotriazol-1-yl)-1,1,3,3-tetramethyluronium-tetrafluoroborate, 41 μl (0.30 mmol) triethylamine and 100 μl conc. Ammonia were stirred into 200 μl dimethylformamide at ambient temperature overnight. The solution was purified by chromatography (RP-HPLC), the corresponding fractions were freeze-dried.

Yield: 3 mg (0.007 mmol=8% of theory)

Analysis: HPLC-MS (method I): Rₜ: 1.53 min, (M+H)⁺: 404

### EXAMPLE 113

### N-{4-[7-(4-morpholin-4-yl-phenyl)-[1,6]naphthyridin-5-ylamino]-cyclohexyl}-acetamide

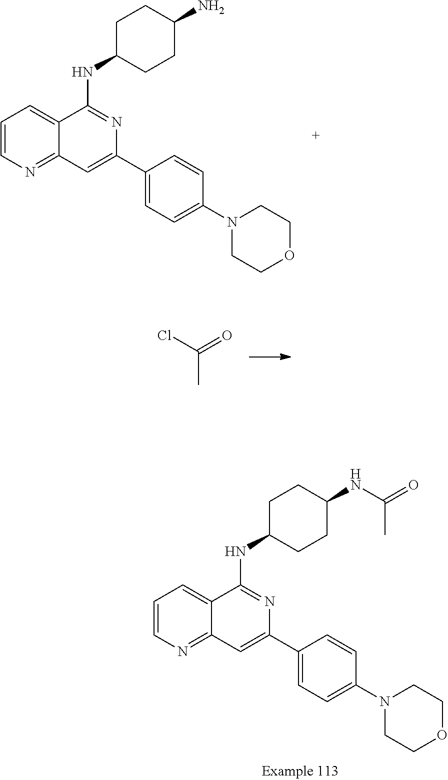

N-[7-(4-morpholin-4-yl-phenyl)-[1,6]naphthyridin-5-yl]-cyclohexane-1,4-diamine was obtained analogously to Example 3 using educt 6.1.

50 mg (0.12 mmol) N-[7-(4-morpholin-4-yl-phenyl)-[1,6]naphthyridin-5-yl]-cyclohexane-1,4-diamine was placed in 0.5 ml dichloromethane and then 63 μl (0.37 mmol) diisopropylethylamine and 7 μl (0.09 mmol) acetyl chloride were added. The reaction mixture was stirred overnight at ambient temperature. Another 5 μl (0.07 mmol) acetyl chloride was added and the mixture was stirred overnight.

It was purified by chromatography (RP-HPLC), the corresponding fractions were freeze-dried.

Yield: 40 mg (0.09 mmol=73% of theory)

Analysis: HPLC-MS (method G): Rₜ: 1.54 min, (M+H)⁺: 446

### EXAMPLE 132

### (4-(5-phenyl-1,6-naphthyridin-7-yl)phenyl)morpholine

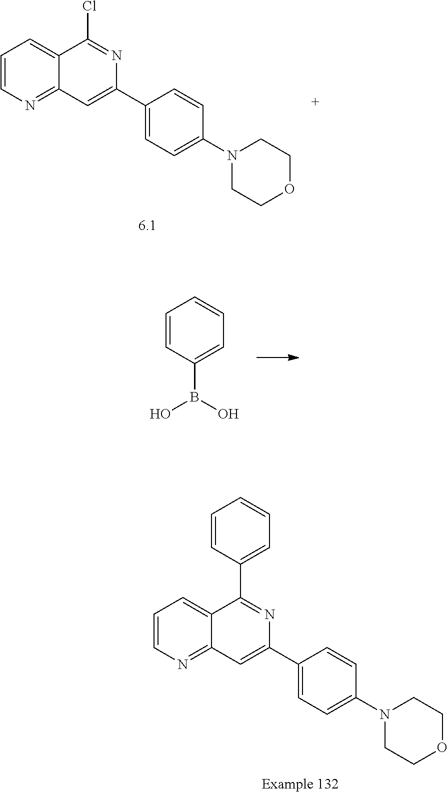

50 mg (0.15 mmol) 6.1 was suspended in 0.5 mL THF, 5.6 mg (0.08 mmol) [1,1′-bis(diphenylphosphino)ferrocene]dichloropalladium(I) and 31.4 mg (0.26 mmol) phenylboric acid, lastly 66.9 mg (0.21 mmol) Cs₂CO₃ dissolved in 0.1 mL water was added dropwise and the mixture was stirred for 20 min at 100° C. under argon. Then it was diluted with methanol and the product was purified by HPLC.

Yield: 38 mg (0.10 mmol=67% of theory)

Analysis: HPLC-MS (method K): Rₜ: 1.97 min, (M+H)⁺: 368

### EXAMPLE 140

### (4-(5-ethyl-[1,6]naphthyridin-7-yl)phenyl)morpholine

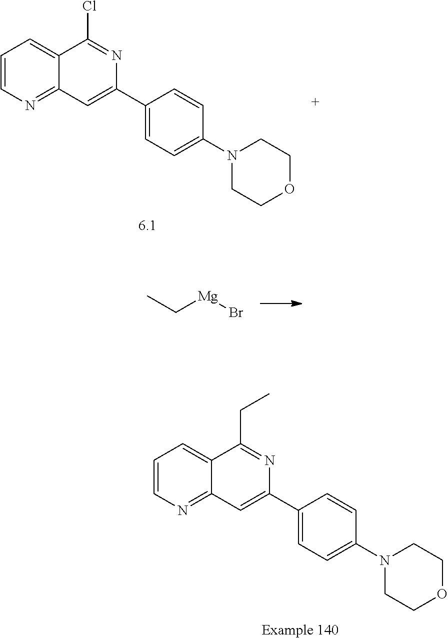

50 mg (0.1 mmol) 6.1, 0.1 mg 1,3-bis(diphenylphosphino)propane nickel(II)chloride were dissolved in 0.5 mL THF and cooled to 0° C. Then 0.465 mL ethylmagnesium bromide (0.4 mmol) solution (1N in THF) was added and the mixture was stirred for 1 h at 0° C. and for 2 h at 25° C. Then a further 0.1 mg 1,3-bis(diphenylphosphino)propane nickel(II)chloride and 0.465 mL ethylmagnesium bromide (0.4 mmol) solution (1N in THF) were added and the mixture was refluxed for 5 h with stirring. Then water was added, the mixture was evaporated down, dissolved again in water and acetonitrile and trifluoroacetic acid, the mixture was filtered and the product was purified by HPLC.

Yield: 5 mg (10% of theory) red solid

Analysis: HPLC-MS (method D): Rₜ: 1.26 min, (M+H)⁺: 320

### EXAMPLE 142

### (4-(5-ethynyl-[1,6]naphthyridin-7-yl)phenyl)morpholine

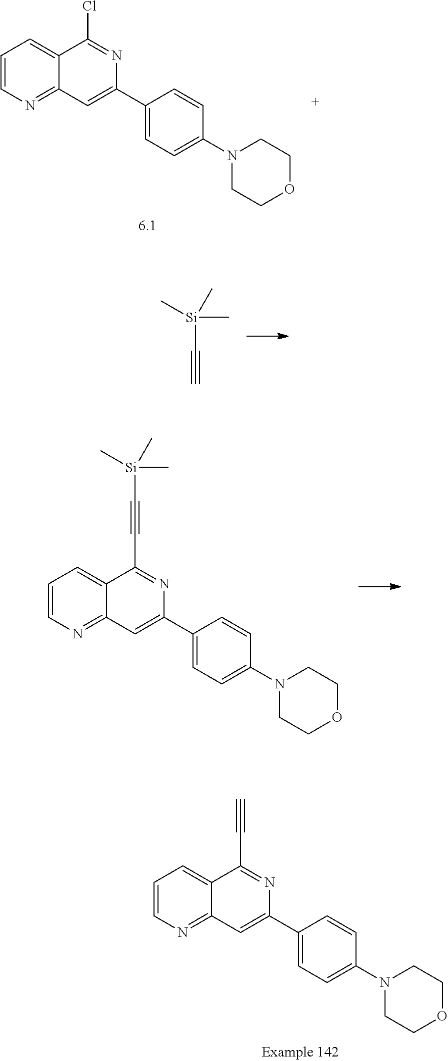

70 mg (0.22 mmol) 6.1, 91 μL (1 mmol) trimethylsiliylacetylene, 0.11 mL (0.645 mmol), 15.5 mg (0.022 mmol) triphenylphosphine palladium(II)chloride and 2.1 mg (0.01 mmol) copper(I)iodide were dissolved in 2 mL acetonitrile and the mixture was stirred for 1.5 h at 80° C. The reaction mixture was diluted with MeOH and dichloromethane, filtered through kieselguhr and evaporated down. The residue was dissolved in dichloromethane and washed successively with 33% ammonia and saturated NaCl solution and the organic phase was evaporated down.

Yield: 100 mg brown solid.

Analysis: HPLC-MS (method D): Rₜ: 1.77 min, (M+H)⁺: 388

100 mg of the brown solid obtained previously was dissolved in 2 mL THF and combined with 0.234 mL (0.23 mmol) tetrabutylammonium fluoride solution (1N in THF) and stirred for 1 h at 25° C. The reaction mixture was diluted with dichloromethane, the organic phase was washed with water, evaporated down and the product was purified by HPLC.

Yield: 10 mg yellow solid (0.032=15% of theoretical)

Analysis: HPLC-MS (method D): Rₜ: 1.39 min, (M+H)⁺: 316

### 4.5 Chromatographic Methods (HPLC-MS Methods)

The Example compounds prepared according to the foregoing synthesis scheme were characterised by the following chromatographic methods, which—if they were carried out—are specified individually in Table 5.

### Method A

Waters ZMD, Alliance 2690/2695 HPLC, Waters 2700 Autosampler, Waters 996/2996 diode array detector

The mobile phase used was:

A: water with 0.10% TFA
B: acetonitrile with 0.10% TFA

| time in min | % A | % B | flow rate in ml/min |
|---|---|---|---|
| 0.0 | 95 | 5 | 1.00 |
| 0.1 | 95 | 5 | 1.00 |
| 3.1 | 2 | 98 | 1.00 |
| 4.5 | 2 | 98 | 1.00 |
| 5.0 | 95 | 5 | 1.00 |

The stationary phase used was an XTerra® column, MS C₁₈ 2.5 μm, 4.6 mm×30 mm (column temperature: constant at 25° C.).

Diode array detection took place in the wavelength range 210-400 nm.

### Method B

Waters ZQ2000, Alliance 2795+2996 HPLC, Waters 2700 Autosampler,

The mobile phase used was:

A: water with 0.10% TFA
B: acetonitrile with 0.10% TFA

| time in min | % A | % B | flow rate in ml/min |
|---|---|---|---|
| 0.0 | 95 | 5 | 1.5 |
| 2.0 | 0 | 100 | 1.5 |
| 3.0 | 0 | 100 | 1.5 |
| 3.4 | 95 | 5 | 1.5 |

The stationary phase used was a X-Terra column MS C18 4.6×50 mm, 3.5 μm (column temperature: constant at 40° C.).

Diode array detection took place in the wavelength range 210-500 nm.

### Method C

Waters ZMD, Alliance 2690/2695 HPLC, Waters 2700 Autosampler, Waters 996/2996 diode array detector

The mobile phase used was:

A: water with 0.10% TFA
B: acetonitrile with 0.10% TFA

| time in min | % A | % B | flow rate in ml/min |
|---|---|---|---|
| 0.00 | 95 | 5 | 2.50 |
| 0.20 | 95 | 5 | 2.50 |
| 1.50 | 2 | 98 | 2.50 |
| 1.70 | 2 | 98 | 2.50 |
| 1.90 | 95 | 5 | 2.50 |
| 2.20 | 95 | 5 | 2.50 |

The stationary phase used was a Merck Chromolith™ Flash RP-18e column, 4.6 mm×25 mm (column temperature: constant at 25° C.).

Diode array detection took place in the wavelength range 210-400 nm.

### Method D

Waters ZMD, Alliance 2690/2695 HPLC, Waters 996/2996 diode array detector

The mobile phase used was:

A: water with 0.10% TFA
B: acetonitrile with 0.10% TFA

| time in min | % A | % B | flow rate in ml/min |
|---|---|---|---|
| 0.00 | 95 | 5 | 2.80 |
| 0.30 | 95 | 5 | 2.80 |
| 1.60 | 2 | 98 | 2.80 |
| 1.90 | 2 | 98 | 2.80 |
| 2.00 | 95 | 5 | 2.50 |

The stationary phase used was a Merck Chromolith™ Flash RP-18e column, 3 mm×100 mm (column temperature: constant at 25° C.).

Diode array detection took place in the wavelength range 210-400 nm.

### Method E

Waters ZQ2000, HP1100 HPLC, Gilson 215 Autosampler,

The mobile phase used was:

A: water with 0.10% TFA
B: acetonitrile with 0.10% TFA

| time in min | % A | % B | flow rate in ml/min |
|---|---|---|---|
| 0.0 | 95 | 5 | 1.5 |
| 2.0 | 0 | 100 | 1.5 |
| 2.5 | 0 | 100 | 1.5 |
| 2.6 | 95 | 5 | 1.5 |

The stationary phase used was a Sunfire column C18 4.6×50 mm, 3.5 μm (column temperature: constant at 40° C.).

Diode array detection took place in the wavelength range 210-500 nm.

### Method F

Agilent 1100 Series LC/MSD SL, DAD: G1315B; MS: G1946D

The mobile phase used was:

A: water with 0.2% formic acid
B: acetonitrile with 0.2% formic acid

| time in min | % A | % B | flow rate in ml/min |
|---|---|---|---|
| 0.0 | 95 | 5 | 1.5 |
| 0.5 | 95 | 5 | 1.5 |
| 4.0 | 5 | 95 | 1.5 |
| 6.0 | 5 | 95 | 1.5 |

The stationary phase used was an Agilent Zorbax column SB-C8, 2.1×50 mm, 3.5 μm

(column temperature: constant at 35° C.).

Diode array detection took place in the wavelength range 190-450 nm.

### Method G

Agilent 1100 Series LC/MSD SL, DAD: G1315B; MS: G1946D

The mobile phase used was:

A: water with 0.2% formic acid
B: acetonitrile with 0.2% formic acid

| time in min | % A | % B | flow rate in ml/min |
|---|---|---|---|
| 0.01 | 95 | 5 | 1.2 |
| 1.50 | 5 | 95 | 1.2 |
| 1.51 | 0 | 100 | 1.2 |
| 2.0 | 0 | 100 | 1.2 |
| 2.01 | 95 | 5 | 1.2 |

stop time: 3.01 min

The stationary phase used was an Agilent Zorbax column SB-C8, 2.1×50 mm, 3.5 μm

(column temperature: constant at 35° C.).

Diode array detection took place in the wavelength range 190-450 nm.

### Method H

Agilent 1100 Series LC/MSD SL, DAD: G1315B; MS: G1946D

The mobile phase used was:

A: 5 mM aqu. NH₄HCO₃— buffer with 20 mM NH₃
B: acetonitrile

| time in min | % A | % B | flow rate in ml/min |
|---|---|---|---|
| 0.01 | 95 | 5 | 1.2 |
| 1.25 | 5 | 95 | 1.2 |
| 2.0 | 5 | 95 | 1.2 |
| 2.01 | 95 | 5 | 1.2 |

stop time: 3.01 min

The stationary phase used was a Waters X-Bridge column C18, 2.1×50 mm, 3.5 μm (column temperature: constant at 35° C.).

Diode array detection took place in the wavelength range 190-450 nm.

### Method I

Agilent 1100 Series LC/MSD SL, DAD: G1315B; MS: G1946D

The mobile phase used was:

A: 5 mM aqueous NH₄HCO₃— buffer with 20 mM NH₃
B: acetonitrile

| time in min | % A | % B | flow rate in ml/min |
|---|---|---|---|
| 0.01 | 95 | 5 | 1.2 |
| 1.25 | 5 | 95 | 1.2 |
| 2.0 | 5 | 95 | 1.2 |
| 2.01 | 95 | 5 | 1.2 |

stop time: 3.01 min

The stationary phase used was a Waters X-Bridge column C18, 2.1×50 mm, 3.5 μm (column temperature: constant at 35° C.).

Diode array detection took place in the wavelength range 190-450 nm.

### Method K

Agilent 1100 Series LC/MSD SL, DAD: G1315B; MS: G1946D

The mobile phase used was:

A: water with 0.2% formic acid
B: acetonitrile with 0.2% formic acid

| time in min | % A | % B | flow rate in ml/min |
|---|---|---|---|
| 0.01 | 95 | 5 | 1.2 |
| 1.50 | 5 | 95 | 1.2 |
| 1.51 | 0 | 100 | 1.2 |
| 2.0 | 0 | 100 | 1.2 |
| 2. 1 | 95 | 5 | 1.2 |

stop time: 3.0 min

The stationary phase used was an Agilent Zorbax column SB-C8, 2.1×50 mm, 3.5 μm

(column temperature: constant at 35° C.).

Diode array detection took place in the wavelength range 190-450 nm.

### Method L

Agilent 1100 Series LC/MSD SL, DAD: G1315B; MS: G1946D

The mobile phase used was:

A: water with 0.2% formic acid
B: acetonitrile with 0.2% formic acid

| time in min | % A | % B | flow rate in ml/min |
|---|---|---|---|
| 0.0 | 95 | 5 | 1.5 |
| 0.25 | 95 | 5 | 1.5 |
| 2.0 | 5 | 95 | 1.5 |
| 3.0 | 5 | 95 | 1.5 |

The stationary phase used was an Agilent Zorbax column SB-C8, 2.1×50 mm, 3.5 μm

(column temperature: constant at 35° C.).

Diode array detection took place in the wavelength range 190-450 nm.

### Method M

Waters ZQ2000, HP1100 HPLC, Gilson 215 Autosampler, Waters 996/2996 diode array detector

The mobile phase used was:

A: water with 0.10% TFA
B: acetonitrile with 0.10% TFA

| time in min | % A | % B | flow rate in ml/min |
|---|---|---|---|
| 0.0 | 95 | 5 | 1.50 |
| 2.0 | 0 | 100 | 1.50 |
| 2.5 | 0 | 100 | 1.50 |
| 2.6 | 95 | 5 | 1.50 |

The stationary phase used was a Sunfire column C₁₈ 3.5 μm, 4.6 mm×50 mm (column temperature: constant at 40° C.).

Diode array detection took place in the wavelength range 210-500 nm.

### 5. EXAMPLES

The following Examples were prepared analogously to the methods of synthesis described above (as indicated in Table 4). These compounds are suitable as SYK inhibitors and have IC₅₀ values of less than or equal to 1 μmol. The inhibitions (in %) at 1 μM of the individual example substances are shown in the following Table of Examples and were determined as follows:

### Syk Kinase Test

Recombinant human Syk was expressed as a fusion protein with an N-terminal GST tag, affinity-purified and deep-frozen at a concentration of approx. 50-100 μM in the test buffer (25 mM HEPES pH7.5; 25 mM MgCl₂; 5 mM MnCl₂; 50 mM KCl; 0.2% BSA; 0.01% CHAPS; 100 μM Na₃VO₄; 0.5 mM DTT) and 10% glycerol at −80° C. until wanted for use.

The catalytic activity of the GST-Syk kinase fusion protein was determined using the Kinase Glo® Luminescence Kinase test of Messrs Promega. In this homogeneous test the amount of ATP remaining after the kinase reaction has been carried out is quantified by a luciferin-luciferase reaction using luminescence. The luminescence signal obtained correlates with the amount of ATP still present and thus correlates inversely with the activity of the protein kinase.

### Method

The test substances were dissolved in 100% DMSO at a concentration of 10 mM and diluted in DMSO to a concentration of 1 mM. All further dilutions of the substances were carried out with 7.5% DMSO in test buffer until a concentration was reached which was 7.5 times above the final test concentration (final concentration of the substances: in normal cases 30 μM to 1 nM). 2 μl aliquots of these dilutions were transferred into a 384-well Optiplate (Perkin Elmer, # 6007290). GST-Syk was diluted to 6.0 nM in the test buffer and 10 μl of this dilution were used in the kinase test (final concentration of Syk=4 nM in a total volume of 15 μl). After 15 minutes' incubation at ambient temperature 3 μl of a mixture of 750 nM ATP and 100 μg/ml poly (L-Glutamic acid L-Tyrosine 4:1), Fluka # 81357) in test buffer were added to each well and then incubation was continued for a further 60 minutes at ambient temperature.

Positive controls are the reaction mixtures that contain no test substance; negative controls are reaction mixtures that contain no kinase.

After 60 minutes, 10 μl Kinase-Glo® solution (Promega, Cat. # V6712) (heated to ambient temperature) are added to each well and incubation is continued for a further 15 minutes at ambient temperature. Then the plates are read in a Microplate Scintillation and Luminescence Counter (PerkinElmer/Wallac: MicroBeta TRILUX 1450 LSC & Luminescence Counter).

### Data Evaluation and Calculation:

The output file of the “MicroBeta TRILUX” is a text file that contains the well number and measurements obtained. For evaluation, the measurement of the negative control was set as 100% inhibition and the measurement of the positive control was set as 0% inhibition. Then from this the % inherent value for the measurement of each substance concentration was calculated using an “MS-Excel-VB macro”. Normally, the % inhibition values calculated are between 100% and 0% inhibition, but in individual cases values may also occur outside these limits. The IC₅₀ values were calculated from the % inhibition values using “GraphPadPrism” software (Version 5) (GraphPad Software Inc.).

TABLE 4
Examples of formula 1
The following Examples of formula 1

1

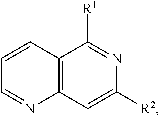

having the following properties were prepared according to the methods of
synthesis described above, wherein X₁ denotes the point where the group R¹ is
linked to the structure of formula 1, and wherein X₂ denotes the point where the
group R² is linked to the structure of formula 1:

| | | | | | % INHB | method of |
|---|---|---|---|---|---|---|
| | | | HPLC-MS | retention | SYK at | prepar- |
| Ex | R¹ | R² | method | time (min) | 1 μM | ation |
| 1 | 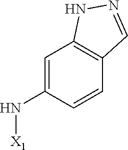 | 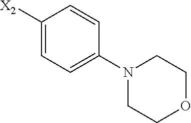 | method A | 2.51 | 82.0 | see de- scription |
| 2 | 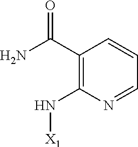 | 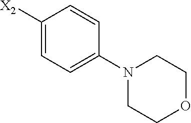 | method A | 2.40 | 72.6 | see de- scription |
| 3 | 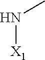 | 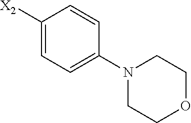 | method C | 1.08 | 101.8 | see de- scription |
| 4 | 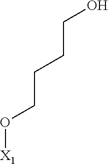 | 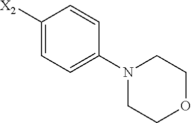 | method C | 1.13 | 94.4 | see de- scription |
| 5 | 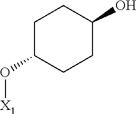 | 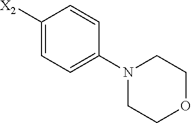 | method C | 1.14 | 103.5 | Analo- gously to Example 4 |
| 6 | 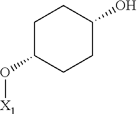 | 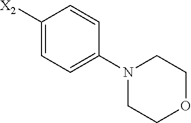 | method C | 1.16 | 98.4 | Analo- gously to Example 4 |
| 7 | 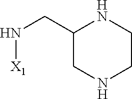 | 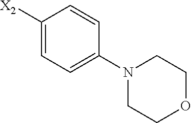 | method D | 1.08 | 81.8 | Analo- gously to Example 3 |
| 8 | 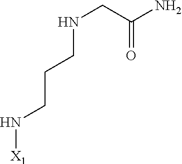 | 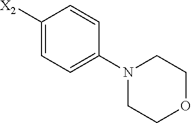 | method D | 1.10 | 78.0 | see de- scription |
| 9 | 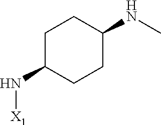 | 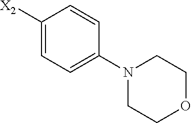 | method D | 1.16 | 86.7 | see de- scription |
| 10 | 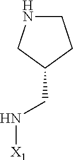 | 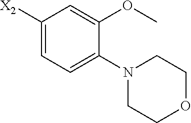 | method D | 1.07 | 98.6 | see de- scription |
| 11 | 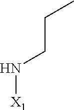 | 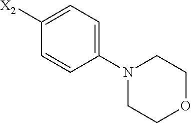 | method E | 1.64 | 62.6 | Analo- gously to Example 62 |
| 12 | 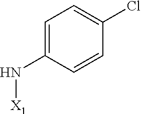 | 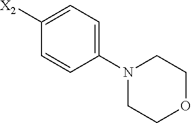 | method E | 1.84 | 82.1 | Analo- gously to Example 62 |
| 13 | 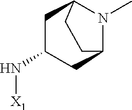 | 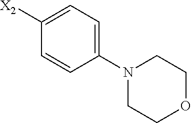 | method E | 1.33 | 74.2 | Analo- gously to Example 62 |
| 14 | 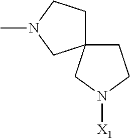 | 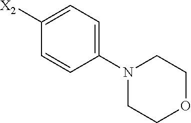 | method E | 1.35 | 88.8 | Analo- gously to Example 62 |
| 15 | 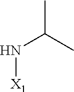 | 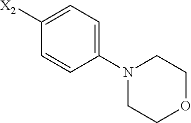 | method E | 1.65 | 91.6 | Analo- gously to Example 62 |
| 16 | 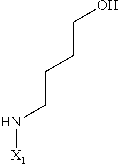 | 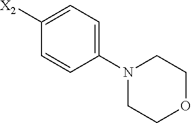 | method E | 1.47 | 93.2 | Analo- gously to Example 62 |
| 17 | 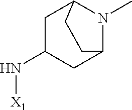 | 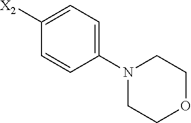 | method E | 1.36 | 76.5 | Analo- gously to Example 62 |
| 18 | 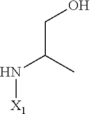 | 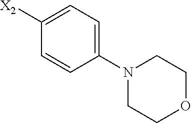 | method E | 1.48 | 99.3 | Analo- gously to Example 62 |
| 19 | 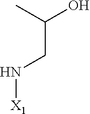 | 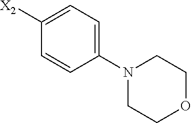 | method E | 1.47 | 93.3 | Analo- gously to Example 62 |
| 20 | 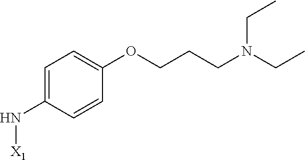 | 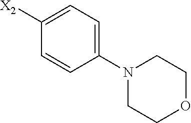 | method E | 1.46 | 77.4 | Analo- gously to Example 62 |
| 21 | 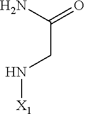 | 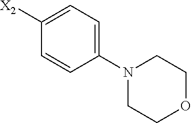 | method E | 1.33 | 87.6 | Analo- gously to Example 62 |
| 22 | 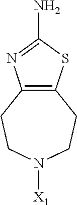 | 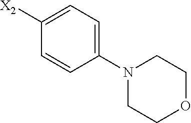 | method E | 1.40 | 72.1 | Analo- gously to Example 62 |
| 23 | 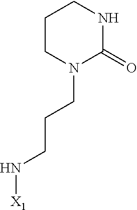 | 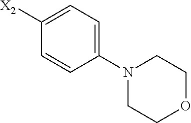 | method E | 1.51 | 83.5 | Analo- gously to Example 62 |
| 24 | 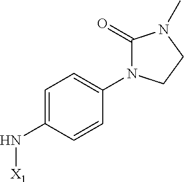 | 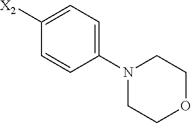 | method E | 1.60 | 81.3 | Analo- gously to Example 62 |
| 25 | 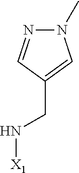 | 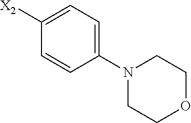 | method E | 1.49 | 76.6 | Analo- gously to Example 62 |
| 26 | 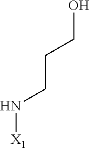 | 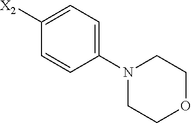 | method E | 1.43 | 87.3 | Analo- gously to Example 62 |
| 27 | 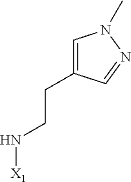 | 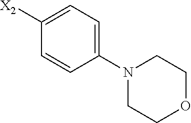 | method E | 1.51 | 85.2 | Analo- gously to Example 62 |
| 28 | 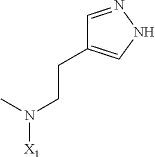 | 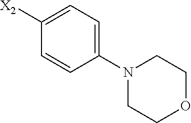 | method E | 1.48 | 85.3 | Analo- gously to Example 62 |
| 29 | 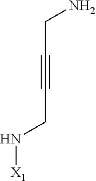 | 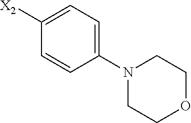 | method E | 1.32 | 93.0 | Analo- gously to Example 62 |
| 30 | 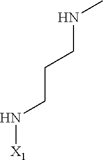 | 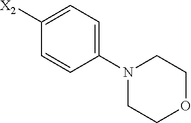 | method E | 1.31 | 82.8 | Analo- gously to Example 62 |
| 31 | 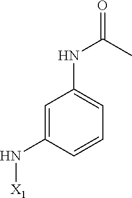 | 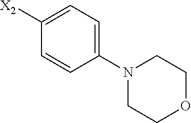 | method E | 1.55 | 99.6 | Analo- gously to Example 62 |
| 32 | 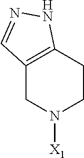 | 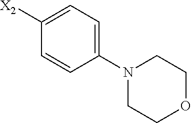 | method E | 1.46 | 88.6 | Analo- gously to Example 62 |
| 33 | 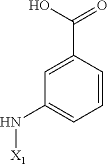 | 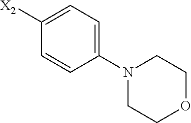 | method E | 1.58 | 79.9 | Analo- gously to Example 62 |
| 34 | 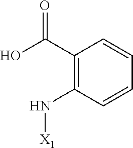 | 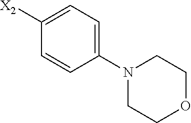 | method E | 1.77 | 100.7 | Analo- gously to Example 62 |
| 35 | 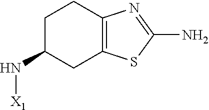 | 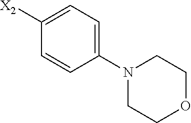 | method E | 1.40 | 97.1 | Analo- gously to Example 62 |
| 36 | 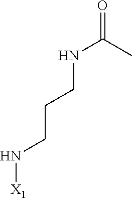 | 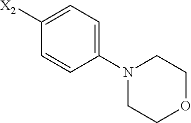 | method E | 1.44 | 86.1 | Analo- gously to Example 62 |
| 37 | 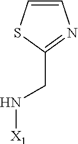 | 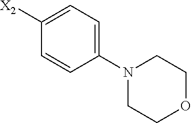 | method E | 1.52 | 72.0 | Analo- gously to Example 62 |
| 38 | 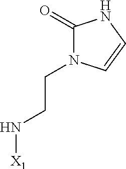 | 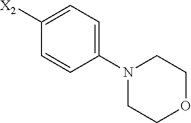 | method E | 1.42 | 81.2 | Analo- gously to Example 62 |
| 39 | 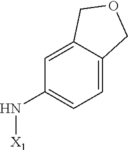 | 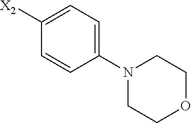 | method E | 1.64 | 99.1 | Analo- gously to Example 62 |
| 40 | 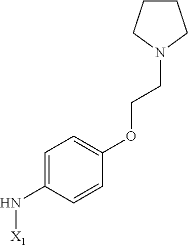 | 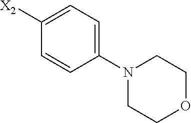 | method E | 1.43 | 80.7 | Analo- gously to Example 62 |
| 41 | 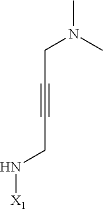 | 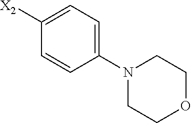 | method E | 1.36 | 76.0 | Analo- gously to Example 62 |
| 42 | 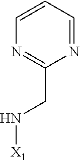 | 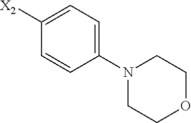 | Method E | 1.43 | 91.8 | Analo- gously to Example 62 |
| 43 | 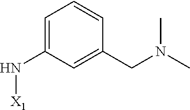 | 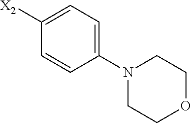 | method E | 1.41 | 87.4 | Analo- gously to Example 62 |
| 44 | 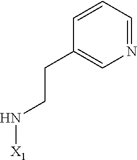 | 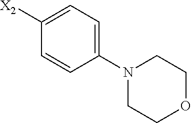 | method E | 1.35 | 90.7 | Analo- gously to Example 62 |
| 45 | 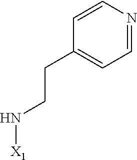 | 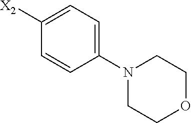 | method E | 1.35 | 92.6 | Analo- gously to Example 62 |
| 46 | 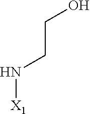 | 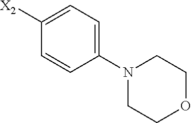 | method E | 1.43 | 92.3 | Analo- gously to Example 62 |
| 47 | 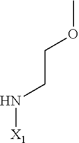 | 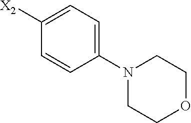 | method E | 1.53 | 109.9 | Analo- gously to Example 62 |
| 48 | 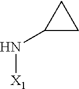 | 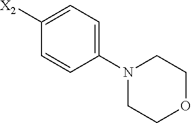 | method E | 1.52 | 95.4 | Analo- gously to Example 62 |
| 49 | 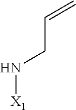 | 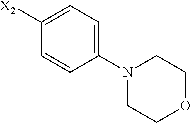 | method E | 1.61 | 85.2 | Analo- gously to Example 62 |
| 50 | 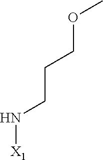 | 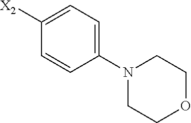 | method E | 1.53 | 86.1 | Analo- gously to Example 62 |
| 51 | 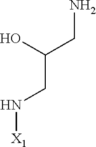 | 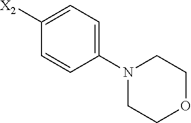 | method E | 1.29 | 89.5 | Analo- gously to Example 62 |
| 52 | 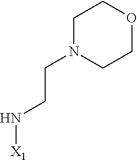 | 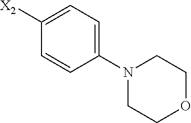 | method E | 1.32 | 86.1 | Analo- gously to Example 62 |
| 53 | 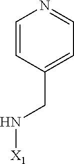 | 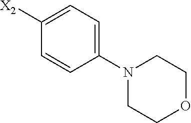 | method E | 1.32 | 82.7 | Analo- gously to Example 62 |
| 54 | 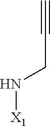 | 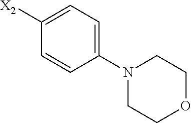 | method E | 1.58 | 91.4 | Analo- gously to Example 62 |
| 55 | 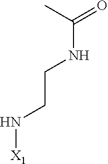 | 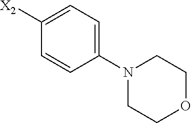 | method E | 1.42 | 80.1 | Analo- gously to Example 62 |
| 56 | 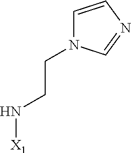 | 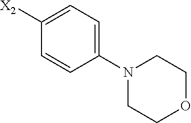 | method E | 1.32 | 82.1 | Analo- gously to Example 62 |
| 57 | 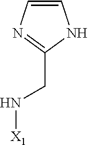 | 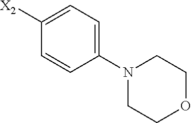 | method E | 1.28 | 88.6 | Analo- gously to Example 62 |
| 58 | 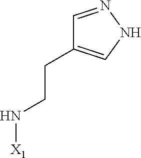 | 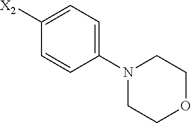 | method E | 1.43 | 100.7 | Analo- gously to Example 62 |
| 59 | 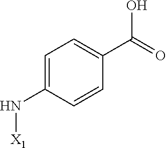 | 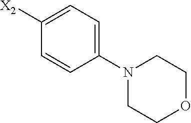 | method E | 1.57 | 103.8 | Analo- gously to Example 62 |
| 60 | 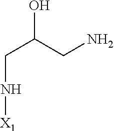 | 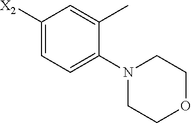 | method D | 1.12 | 92.0 | Analo- gously to Example 3 |
| 61 | 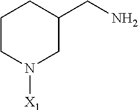 | 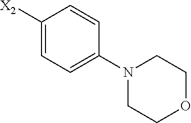 | method D | 1.15 | 73.1 | Analo- gously to Example 10 |
| 62 | 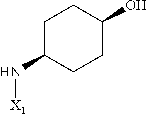 | 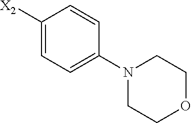 | method D | 1.24 | 76.8 | see de- scription |
| 63 | 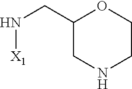 | 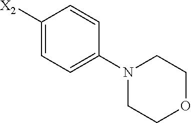 | method D | 1.11 | 85.4 | see de- scription |
| 64 | 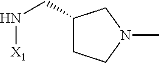 | 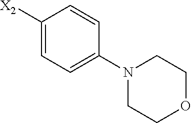 | method D | 1.15 | 67.7 | Analo- gously to Example 62 |
| 65 | 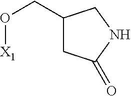 | 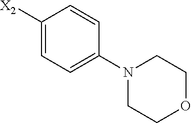 | method D | 1.18 | 91.5 | see de- scription |
| 66 | 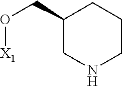 | 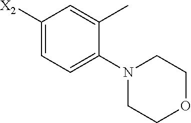 | method D | 1.22 | 102.8 | see de- scription |
| 67 | 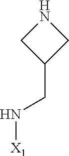 | 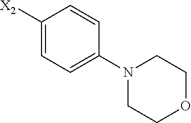 | method D | 1.11 | 81.6 | Analo- gously to Example 63 |
| 68 | 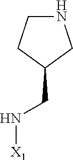 | 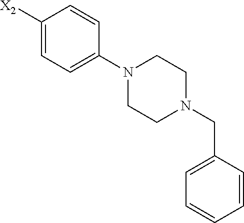 | method D | 1.12 | 91.5 | Analo- gously to Example 63 |
| 69 | 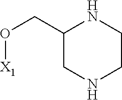 | 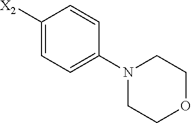 | method D | 1.11 | 92.9 | Analo- gously to Example 66 |
| 70 | 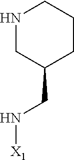 | 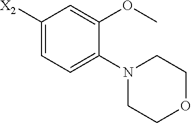 | method D | 1.11 | 87.4 | Analo- gously to Example 10 |
| 71 | 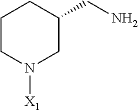 | 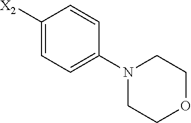 | method D | 1.15 | 78.0 | Analo- gously to Example 10 |
| 72 | 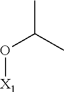 | 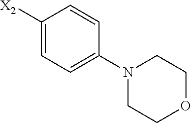 | method D | 1.45 | 84.8 | Analo- gously to Example 4 |
| 73 | 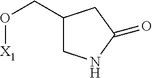 | 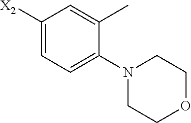 | method D | 1.27 | 102.8 | Analo- gously to Example 4 |
| 74 | 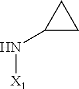 | 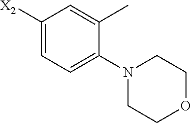 | method D | 1.30 | 86.3 | Analo- gously to Example 3 |
| 75 | 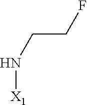 | 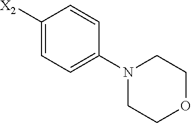 | method D | 1.27 | 86.1 | Analo- gously to Example 62 |
| 76 | 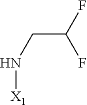 | 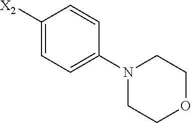 | method D | 1.31 | 86.5 | Analo- gously to Example 62 |
| 77 | 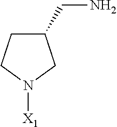 | 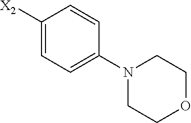 | method D | 1.11 | 104.2 | Analo- gously to Example 63 |
| 78 | 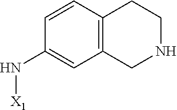 | 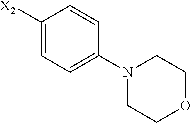 | method D | 1.16 | 77.0 | Analo- gously to Example 63 |
| 79 | 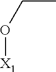 | 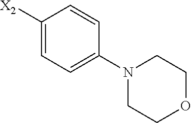 | method D | 1.40 | 82.9 | see de- scription |
| 80 | 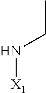 | 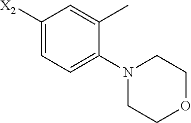 | method D | 1.33 | 89.8 | Analo- gously to Example 3 |
| 81 | 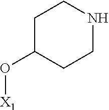 | 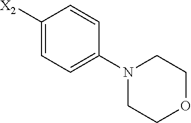 | method D | 1.13 | 89.1 | Analo- gously to Example 66 |
| 82 | 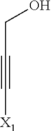 | 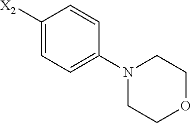 | method D | 1.26 | 87.1 | see de- scription |
| 83 | 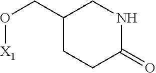 | 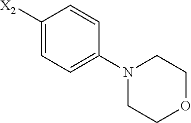 | method D | 1.24 | 82.1 | Analo- gously to Example 4 |
| 84 | 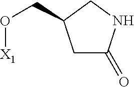 | 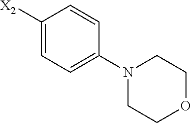 | method D | 1.20 | 90.1 | see de- scription |
| 85 | 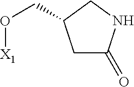 | 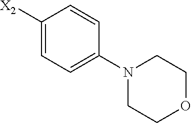 | method D | 1.20 | 97.5 | Analo- gously to Example 84 |
| 86 | 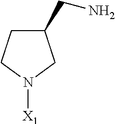 | 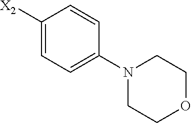 | method D | 1.11 | 96.4 | Analo- gously to Example 63 |
| 87 | 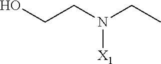 | 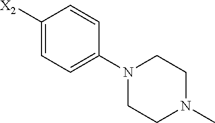 | method F | 1.56 | 75.3 | Analo- gously to Example 3 |
| 88 | 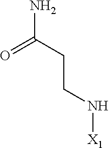 | 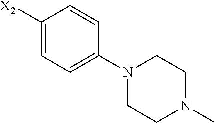 | method F | 1.40 | 90.8 | Analo- gously to Example 3 |
| 89 | 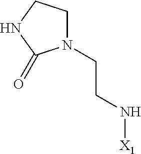 | 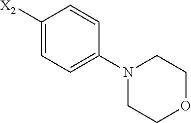 | method D | 1.45 | 84.8 | Analo- gously to Example 4 |
| 90 | 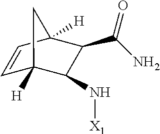 | 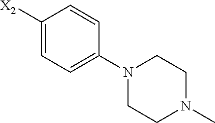 | method L | 1.63 | 110.1 | Analo- gously to Example 3 |
| 91 | 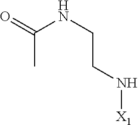 | 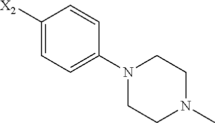 | method L | 1.47 | 56.6 | Analo- gously to Example 3 |
| 92 | 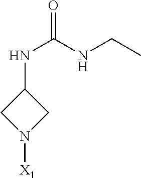 | 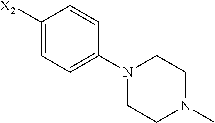 | method L | 1.51 | 77.3 | see de- scription |
| 93 | 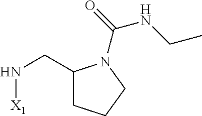 | 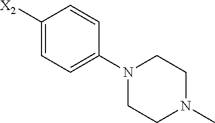 | method F | 1.59 | 91.5 | see de- scription |
| 94 | 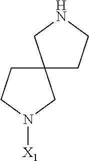 | 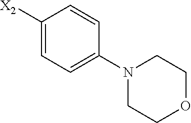 | method G | 1.51 | 102.0 | Analo- gously to Example 3 |
| 95 | 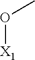 | 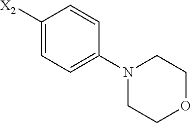 | method D | 1.32 | 89.3 | Analo- gously to Example 79 |
| 96 | 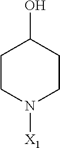 | 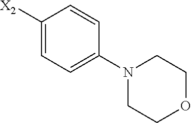 | method H | 1.60 | 72.5 | Analo- gously to Example 3 |
| 97 | 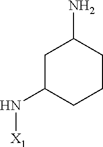 | 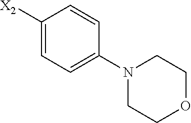 | method H | 1.63 | 93.8 | Analo- gously to Example 3 |
| 98 | 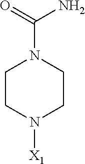 | 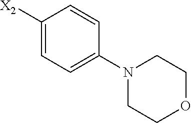 | method I | 1.53 | 86.8 | see de- scription |
| 99 | 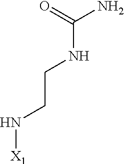 | 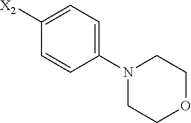 | method H | 1.40 | 91.4 | Analo- gously to Example 98 |
| 100 | 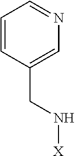 | 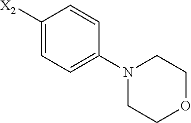 | method G | 1.35 | 105.9 | Analo- gously to Example 3 |
| 101 | 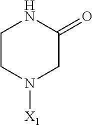 | 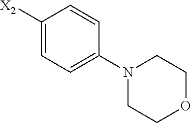 | method G | 1.52 | 75.5 | Analo- gously to Example 3 |
| 102 | 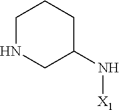 | 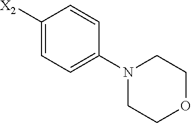 | method I | 1.59 | 71.6 | Analo- gously to Example 10 |
| 103 | 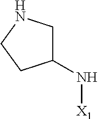 | 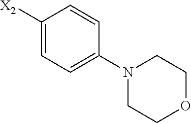 | method G | 1.30 | 83.1 | Analo- gously to Example 10 |
| 104 | 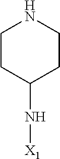 | 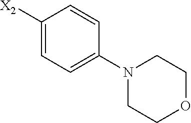 | method G | 1.28 | 107.2 | Analo- gously to Example 10 |
| 105 | 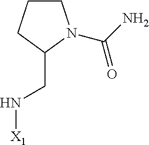 | 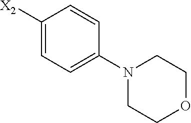 | method I | 1.57 | 82.0 | Analo- gously to Example 98 |
| 106 | 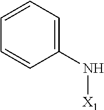 | 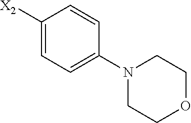 | method K | 1.78 | 85.0 | Analo- gously to Example 1 |
| 107 | 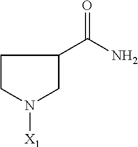 | 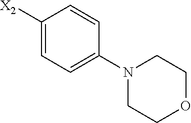 | method I | 1.53 | 95.3 | see de- scription |
| 108 | 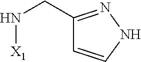 | 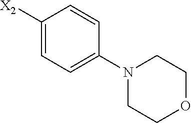 | method I | 1.60 | 105.3 | Analo- gously to Example 3 |
| 109 | 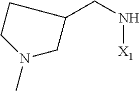 | 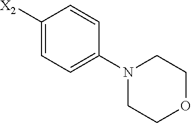 | method I | 1.70 | 86.5 | Analo- gously to Example 3 |
| 110 | 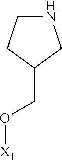 | 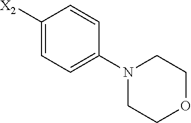 | method I | 1.72 | 99.0 | Analo- gously to Example 66 |
| 111 | 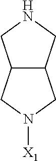 | 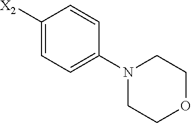 | method I | 1.67 | 79.8 | Analo- gously to Example 10 |
| 112 | 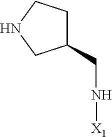 | 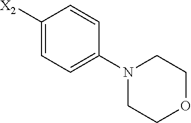 | method K | 1.36 | 123.2 | Analo- gously to Example 10 |
| 113 | 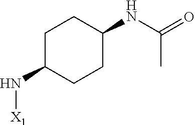 | 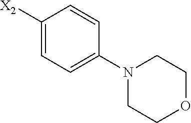 | method G | 1.54 | 94.7 | see de- scription |
| 114 | 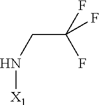 | 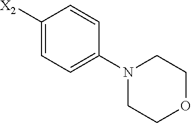 | method D | 1.36 | 84.2 | Analo- gously to Example 62 |
| 115 | 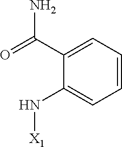 | 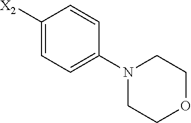 | method E | 1.91 | 93.2 | Analo- gously to Example 62 |
| 116 | 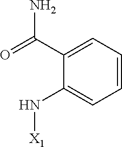 | 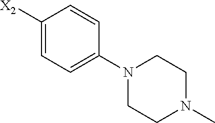 | method D | 1.16 | 86.9 | Analo- gously to Example 4 |
| 117 | 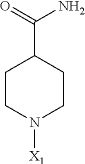 | 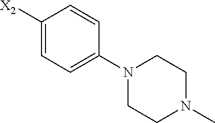 | method F | 1.57 | 77.9 | Analo- gously to Example 3 |
| 118 | 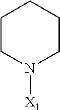 | 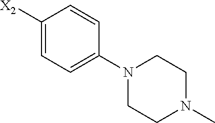 | method F | 1.81 | 36.1 | Analo- gously to Example 3 |
| 119 | 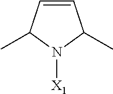 | 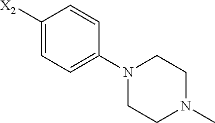 | method F | 1.78 | 71.7 | Analo- gously to Example 3 |
| 120 | 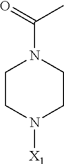 | 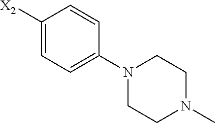 | method F | 1.68 | 73.1 | Analo- gously to Example 3 |
| 121 | 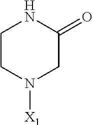 | 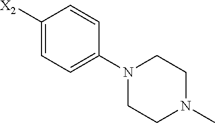 | method F | 1.60 | 103 | Analo- gously to Example 3 |
| 122 | 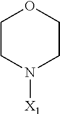 | 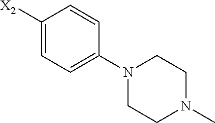 | method F | 1.66 | 80.6 | Analo- gously to Example 3 |
| 123 | 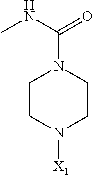 | 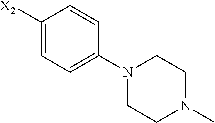 | method F | 1.61 | 66 | Analo- gously to Example 3 |
| 124 | 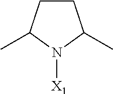 | 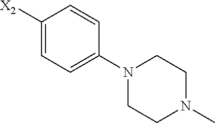 | method F | 1.75 | 75.9 | Analo- gously to Example 3 |
| 125 | 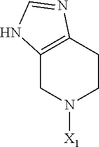 | 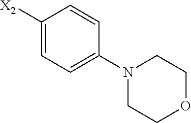 | method I | 1.57 | 96.4 | Analo- gously to Example 3 |
| 126 | 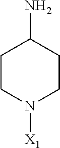 | 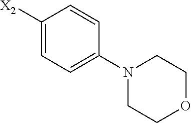 | method H | 1.61 | 80.1 | Analo- gously to Example 3 |
| 127 | 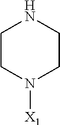 | 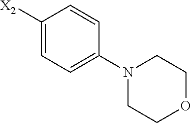 | method I | 1.58 | 45.9 | Analo- gously to Example 3 |
| 128 | 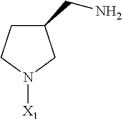 | 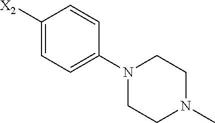 | method D | 1.03 | 95.2 | Analo- gously to Example 10 |
| 129 | 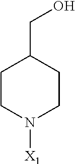 | 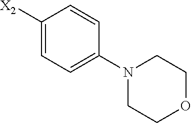 | method H | 1.62 | 74.5 | Analo- gously to Example 3 |
| 130 | 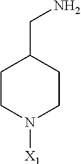 | 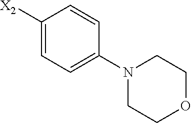 | method H | 1.66 | 91.1 | Analo- gously to Example 3 |
| 131 | 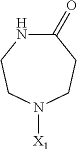 | 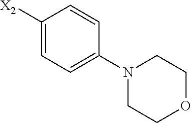 | method K | 1.59 | 63.4 | Analo- gously to Example 3 |
| 132 | 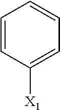 | 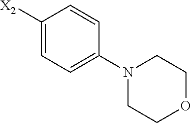 | method K | 1.97 | 55.2 | see de- scription |
| 133 | 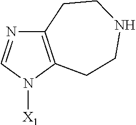 | 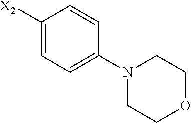 | | | 52.3 | Analo- gously to Example 4 |
| 134 | 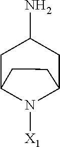 | 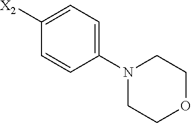 | method M | 1.37 | 60.3 | Analo- gously to Example 62 |
| 135 | 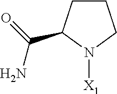 | 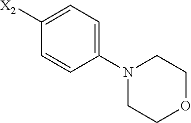 | method M | 1.37 | 60.7 | Analo- gously to Example 62 |
| 136 | 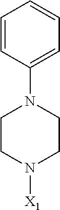 | 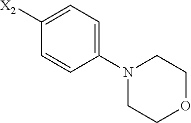 | method M | 1.71 | 53.5 | Analo- gously to Example 62 |
| 137 | 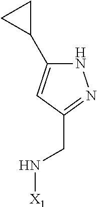 | 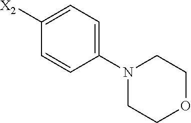 | method M | 1.48 | 80.1 | Analo- gously to Example 62 |
| 138 | 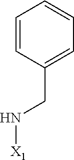 | 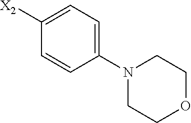 | method M | 1.72 | 66.9 | Analo- gously to Example 62 |
| 139 | 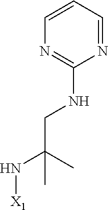 | 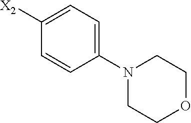 | method M | 1.51 | 59.1 | Analo- gously to Example 62 |
| 140 | 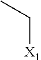 | 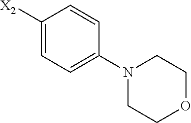 | method D | 1.26 | 43.3 | see de- scription |
| 141 | 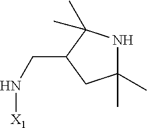 | 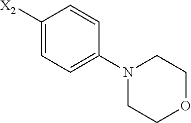 | method D | 1.19 | 72.9 | Analo- gously to Example 62 |
| 142 | 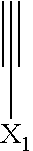 | 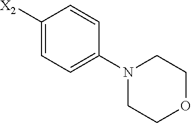 | method D | 1.39 | 70.3 | see de- scription |
| 143 | 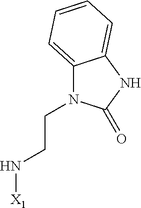 | 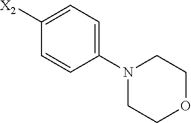 | method M | 1.55 | 55.1 | Analo- gously to Example 62 |
| 144 | 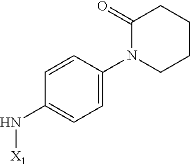 | 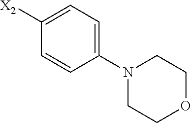 | method M | 1.59 | 80.2 | Analo- gously to Example 62 |
| 145 | 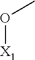 | 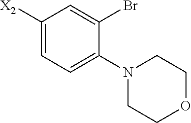 | method D | 1.53 | 63.5 | Analo- gously to Example 66 |

### 6. INDICATIONS

As has been found, the compounds of formula 1 are characterised by their range of applications in the therapeutic field. Particular mention should be made of those applications for which the compounds of formula 1 according to the invention are preferably used on the basis of their pharmaceutical activity as SYK-inhibitors. Examples include respiratory complaints, allergic diseases, osteoporosis, gastrointestinal diseases or complaints, immune or autoimmune diseases, allergic diseases, inflammatory diseases, e.g. inflammatory diseases of the joints, skin and eyes and diseases of the peripheral or central nervous system.

Particular mention should be made of the prevention and treatment of respiratory tract and pulmonary diseases which are accompanied by increased mucus production, inflammation and/or obstructive diseases of the airways. Examples of these include asthma, paediatrich asthma, ARDS (Adult Respiratory Distress Syndrome), acute, allergic or chronic bronchitis, chronic obstructive bronchitis (COPD) (including the treatment of Rhinovirus-induced exacerbations), coughs, allergic rhinitis or sinusitis, allergic rhinoconjunctivitis, chronic rhinitis or sinusitis, alveolitis, farmers' lung, hyperreactive airways, infectious bronchitis or pneumonitis, bronchiectasis, pulmonary fibrosis, bronchial oedema, pulmonary oedema, pneumonia or interstitial pneumonia triggered by various causes such as aspiration, inhalation of toxic gases or bronchitis, pneumonia or interstitial pneumonia triggered by cardiac insufficiency, radiation, chemotherapy, cystic fibrosis or mucoviscidosis, alpha1-antitrypsin deficiency.

The compounds according to the invention are preferably also suitable for the treatment of allergic diseases such as for example allergic rhinitis, allergic rhinoconjunctivitis, allergic conjunctivitis, and contact dermatitis, urticaria/angiooedema and allergic dermatitis.

Mention should also preferably be made of the treatment of inflammatory diseases of the gastrointestinal tract. Examples of these are Crohn's disease and ulcerative colitis.

The compounds according to the invention are preferably also suitable for the treatment of inflammatory diseases of the joints or inflammatory diseases of the skin and eyes. Examples of these are rheumatoid arthritis, antibody-based glomerulonephritis, psoriasis, Kawasaki syndrome, coeliac disease (sprue) and Wegener's granulomatosis.

The compounds according to the invention are preferably also suitable for the treatment of autoimmune diseases. Examples of these are hepatitis (autoimmune-based), lupus erythematodes, anti-phospholipid syndrome, Berger's disease, Evans's syndrome, immunohaemolytic anaemia, ITP (idiopathic thrombocytopenic purpura; adult, neonatal and paediatric), myasthenia gravis, Sjögren's syndrome and sclerodermy.

The compounds according to the invention are preferably also suitable for the treatment of B-cell lymphomas.

Mention may preferably also be made of the prevention and treatment of diseases of the peripheral or central nervous system. Examples of these are acute and chronic multiple sclerosis or non-familial lateral sclerosis.

Mention may preferably also be made of the prevention and treatment of osteoporotic diseases such as for example disease-associated osteopenia, osteoporosis and osteolytic diseases.

The present invention relates particularly preferably to the use of compounds of formula 1 for preparing a pharmaceutical composition for the treatment of diseases selected from among asthma, COPD, allergic rhinitis, Adult Respiratory Distress Syndrome, bronchitis, allergic dermatitis, contact dermatitis, ITP, rheumatoid arthritis and allergic rhinoconjunctivitis.

Most preferably, the compounds of formula 1 may be used for the treatment of a disease selected from among asthma, allergic rhinitis, rheumatoid arthritis, allergic dermatitis and COPD.

### 7. COMBINATIONS

The compounds of formula 1 may be used on their own or in conjunction with other active substances of formula 1 according to the invention. The compounds of formula 1 may optionally also be used in conjunction with other pharmacologically active substances. Preferably the active substances used here may be selected for example from among the # betamimetics, anticholinergics, corticosteroids, PDE4-inhibitors, LTD4-antagonists, EGFR-inhibitors, MRP4-inhibitors, dopamine agonists, H1-antihistamines, PAF-antagonists, iNos-inhibitors, PI3-kinase-inhibitors, CCR3-antagonists, CCR2-antagonists, CCR1-antagonists, IKK2-inhibitors, A2a agonists, alpha-4-integrin-inhibitors, CRTH2-antagonists, histamine 1, combined H1/H3-antagonists, p38 kinase inhibitors, methylxanthines, ENaC-inhibitors, CXCR1-antagonists, CXCR2-antagonists, ICE-inhibitors, LTB4-antagonists, 5-LO antagonists, FLAP-antagonists. LTB4-antagonists; cromoglycine, dissociated glucocorticoid mimetics, anti-TNF-antibodies, anti-GM-CSF antibodies, anti-CD46-antibodies, anti-IL-1-antibodies, anti-IL-2-antibodies, anti-IL-4-antibodies, anti-IL-5-antibodies, anti-IL-13-antibodies, anti-IL-4/IL-13-antibodies, or double or triple combinations thereof, such as for example combinations of compounds of formula 1 with one or two compounds selected from among the
betamimetics, corticosteroids, SYK-inhibitors of formula 1, EGFR-inhibitors and PDE4-antagonists,
anticholinergics, betamimetics, corticosteroids, SYK-inhibitors of formula 1, EGFR-inhibitors and PDE4-antagonists,
PDE4-inhibitors, corticosteroids, EGFR-inhibitors and SYK-inhibitors of formula
EGFR-inhibitors, PDE4-inhibitors and SYK-inhibitors of formula 1
EGFR-inhibitors and SYK-inhibitors of formula 1
SYK-inhibitors of formula 1, betamimetics and anticholinergics
anticholinergics, betamimetics, corticosteroids, PDE4-inhibitors and SYK-inhibitors of formula 1.

Combinations of three active substances each taken from one of the above-mentioned categories of compounds are also an object of the invention.

Suitable betamimetics used are preferably compounds selected from among albuterol, bambuterol, bitolterol, broxaterol, carbuterol, carmoterol, indacaterol, clenbuterol, fenoterol, formoterol, arformoterol, zinterol, hexoprenaline, ibuterol, isoetharine, isoprenaline, levosalbutamol, mabuterol, meluadrine, metaproterenol, orciprenaline, pirbuterol, procaterol, reproterol, rimiterol, ritodrine, salmeterol, salmefamol, soterenol, sulphonterol, tiaramide, terbutaline, tolubuterol, CHF-1035, HOKU-81, KUL-1248, 3-(4-{6-[2-hydroxy-2-(4-hydroxy-3-hydroxymethyl-phenyl)-ethylamino]-hexyloxy}-butyl)-benzyl-sulphonamide, 5-[2-(5,6-diethyl-indan-2-ylamino)-1-hydroxy-ethyl]-8-hydroxy-1H-quinolin-2-one, 4-hydroxy-7-[2-{[2-{[3-(2-phenylethoxy)propyl]sulphonyl}ethyl]amino}ethyl]-2(3H)-benzothiazolone, 1-(2-fluoro-4-hydroxyphenyl)-2-[4-(1-benzimidazolyl)-2-methyl-2-butylamino]ethanol, 1-[3-(4-methoxybenzyl-amino)-4-hydroxyphenyl]-2-[4-(1-benzimidazolyl)-2-methyl-2-butylamino]ethanol, 1-[2H-5-hydroxy-3-oxo-4H-1,4-benzoxazin-8-yl]-2-[3-(4-N,N-dimethylaminophenyl)-2-methyl-2-propylamino]ethanol, 1-[2H-5-hydroxy-3-oxo-4H-1,4-benzoxazin-8-yl]-2-[3-(4-methoxyphenyl)-2-methyl-2-propylamino]ethanol, 1-[2H-5-hydroxy-3-oxo-4H-1,4-benzoxazin-8-yl]-2-[3-(4-n-butyloxyphenyl)-2-methyl-2-propylamino]ethanol, 1-[2H-5-hydroxy-3-oxo-4H-1,4-benzoxazin-8-yl]-2-{4-[3-(4-methoxyphenyl)-1,2,4-triazol-3-yl]-2-methyl-2-butylamino}ethanol, 5-hydroxy-8-(1-hydroxy-2-isopropylaminobutyl)-2H-1,4-benzoxazin-3-(4H)-one, 1-(4-amino-3-chloro-5-trifluoromethylphenyl)-2-tert.-butylamino)ethanol, 6-hydroxy-8-{1-hydroxy-2-[2-(4-methoxy-phenyl)-1,1-dimethyl-ethylamino]-ethyl}-4H-benzo[1,4]oxazin-3-one, 6-hydroxy-8-{1-hydroxy-2-[2-(ethyl 4-phenoxy-acetate)-1,1-dimethyl-ethylamino]-ethyl}-4H-benzo[1,4]oxazin-3-one, 6-hydroxy-8-{1-hydroxy-2-[2-(4-phenoxy-acetic acid)-1,1-dimethyl-ethylamino]-ethyl}-4H-benzo[1,4]oxazin-3-one, 8-{2-[1,1-dimethyl-2-(2,4,6-trimethylphenyl)-ethylamino]-1-hydroxy-ethyl}-6-hydroxy-4H-benzo[1,4]oxazin-3-one, 6-hydroxy-8-{1-hydroxy-2-[2-(4-hydroxy-phenyl)-1,1-dimethyl-ethylamino]-ethyl}-4H-benzo[1,4]oxazin-3-one, 6-hydroxy-8-{1-hydroxy-2-[2-(4-isopropyl-phenyl)-1,1-dimethyl-ethylamino]-ethyl}-4H-benzo[1,4]oxazin-3-one, 8-{2-[2-(4-ethyl-phenyl)-1,1-dimethyl-ethylamino]-1-hydroxy-ethyl}-6-hydroxy-4H-benzo[1,4]oxazin-3-one, 8-{2-[2-(4-ethoxy-phenyl)-1,1-dimethyl-ethylamino]-1-hydroxy-ethyl}-6-hydroxy-4H-benzo[1,4]oxazin-3-one, 4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric acid, 8-{2-[2-(3,4-difluoro-phenyl)-1,1-dimethyl-ethylamino]-1-hydroxy-ethyl}-6-hydroxy-4H-benzo[1,4]oxazin-3-one and 1-(4-ethoxy-carbonylamino-3-cyano-5-fluorophenyl)-2-(tert.-butylamino)ethanol, optionally in the form of the racemates, enantiomers, diastereomers and optionally in the form of the pharmacologically acceptable acid addition salts, solvates or hydrates thereof.

Of these betamimetics the particularly preferred ones according to the invention are formoterol, salmeterol, 3-(4-{6-[2-hydroxy-2-(4-hydroxy-3-hydroxymethyl-phenyl)-ethylamino]-hexyloxy}-butyl)-benzenesulphonamide, 6-hydroxy-8-{1-hydroxy-2-[2-(4-methoxy-phenyl)-1,1-dimethyl-ethylamino]-ethyl}-4H-benzo[1,4]oxazin-3-one, 6-hydroxy-8-{1-hydroxy-2-[2-(ethyl 4-phenoxy-acetate)-1,1-dimethyl-ethylamino]-ethyl}-4H-benzo[1,4]oxazin-3-one, 6-hydroxy-8-{1-hydroxy-2-[2-(4-phenoxy-acetic acid)-1,1-dimethyl-ethylamino]-ethyl}-4H-benzo[1,4]oxazin-3-one, 8-{2-[1,1-dimethyl-2-(2,4,6-trimethylphenyl)-ethylamino]-1-hydroxy-ethyl}-6-hydroxy-4H-benzo[1,4]oxazin-3-one, 6-hydroxy-8-{1-hydroxy-2-[2-(4-hydroxy-phenyl)-1,1-dimethyl-ethylamino]-ethyl}-4H-benzo[1,4]oxazin-3-one, 6-hydroxy-8-{1-hydroxy-2-[2-(4-isopropyl-phenyl)-1,1 dimethyl-ethylamino]-ethyl}-4H-benzo[1,4]oxazin-3-one, 8-{2-[2-(4-ethyl-phenyl)-1,1-dimethyl-ethylamino]-1-hydroxy-ethyl}-6-hydroxy-4H-benzo[1,4]oxazin-3-one, 8-{2-[2-(4-ethoxy-phenyl)-1,1-dimethyl-ethylamino]-1-hydroxy-ethyl}-6-hydroxy-4H-benzo[1,4]oxazin-3-one, 4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric acid, 8-{2-[2-(3,4-difluoro-phenyl)-1,1-dimethyl-ethylamino]-1-hydroxy-ethyl}-6-hydroxy-4H-benzo[1,4]oxazin-3-one and 5-[2-(5,6-diethyl-indan-2-ylamino)-1-hydroxy-ethyl]-8-hydroxy-1H-quinoline-2-one, optionally in the form of the racemates, enantiomers, diastereomers and optionally in the form of the pharmacologically acceptable acid addition salts, solvates or hydrates thereof.

According to the invention the acid addition salts of the betamimetics are preferably selected from among the hydrochloride, hydrobromide, hydroiodide, hydrosulphate, hydrophosphate, hydromethanesulphonate, hydronitrate, hydromaleate, hydroacetate, hydrocitrate, hydrofumarate, hydrotartrate, hydrooxalate, hydrosuccinate, hydrobenzoate and hydro-p-toluenesulphonate, preferably the hydrochloride, hydrobromide, hydrosulphate, hydrophosphate, hydrofumarate and hydromethanesulphonate. Of the above-mentioned acid addition salts the salts of hydrochloric acid, methanesulphonic acid, benzoic acid and acetic acid are particularly preferred according to the invention.

The anticholinergics used are preferably compounds selected from among the tiotropium salts, oxitropium salts, flutropium salts, ipratropium salts, glycopyrronium salts, aclidinium bromide, trospium salts, tropenol 2,2-diphenylpropionate methobromide, scopine 2,2-diphenylpropionate methobromide, scopine 2-fluoro-2,2-diphenylacetate methobromide, tropenol 2-fluoro-2,2-diphenylacetate methobromide, tropenol 3,3′,4,4′-tetrafluorobenzilate methobromide, scopine 3,3′,4,4′-tetrafluorobenzilate methobromide, tropenol 4,4′-difluorobenzilate methobromide, scopine 4,4′-difluorobenzilate methobromide, tropenol 3,3′-difluorobenzilate methobromide, scopine 3,3′-difluorobenzilate methobromide, tropenol 9-hydroxy-fluorene-9-carboxylate-methobromide, tropenol 9-fluoro-fluorene-9-carboxylate-methobromide, scopine 9-hydroxy-fluoren-9-carboxylate methobromide, scopine 9-fluoro-fluorene-9-carboxylate methobromide, tropenol 9-methyl-fluorene-9-carboxylate methobromide, scopine 9-methyl-fluorene-9-carboxylate methobromide, cyclopropyltropine benzilate methobromide, cyclopropyltropine 2,2-diphenylpropionate methobromide, cyclopropyltropine 9-hydroxy-xanthene-9-carboxylate methobromide, cyclopropyltropine 9-methyl-fluorene-9-carboxylate methobromide, cyclopropyltropine 9-methyl-xanthene-9-carboxylate methobromide, cyclopropyltropine 9-hydroxy-fluorene-9-carboxylate methobromide, methyl cyclopropyltropine 4,4′-difluorobenzilate methobromide, tropenol 9-hydroxy-xanthene-9-carboxylate-methobromide, scopine 9-hydroxy-xanthene-9-carboxylate methobromide, tropenol 9-methyl-xanthene-9-carboxylate methobromide, scopine 9-methyl-xanthene-9-carboxylate methobromide, tropenol 9-ethyl-xanthene-9-carboxylate methobromide, tropenol 9-difluoromethyl-xanthene-9-carboxylate methobromide, scopine 9-hydroxymethyl-xanthene-9-carboxylate methobromide, optionally in the form of the solvates or hydrates thereof.

In the above-mentioned salts the cations tiotropium, oxitropium, flutropium, ipratropium, glycopyrronium, aclidinium and trospium are the pharmacologically active ingredients. As anions, the above-mentioned salts may preferably contain chloride, bromide, iodide, sulphate, phosphate, methanesulphonate, nitrate, maleate, acetate, citrate, fumarate, tartrate, oxalate, succinate, benzoate or p-toluenesulphonate, while chloride, bromide, iodide, sulphate, methanesulphonate or p-toluenesulphonate are preferred as counter-ions. Of all the salts, the chlorides, bromides, iodides and methanesulphonate are particularly preferred.

Of particular importance is tiotropium bromide. In the case of tiotropium bromide the pharmaceutical combinations according to the invention preferably contain it in the form of the crystalline tiotropium bromide monohydrate, which is known from WO 02/30928. If the tiotropium bromide is used in anhydrous form in the pharmaceutical combinations according to the invention, it is preferable to use anhydrous crystalline tiotropium bromide, which is known from WO 03/000265.

Corticosteroids used here are preferably compounds selected from among prednisolone, prednisone, butixocortpropionate, flunisolide, beclomethasone, triamcinolone, budesonide, fluticasone, mometasone, ciclesonide, rofleponide, dexamethasone, betamethasone, deflazacort, RPR-106541, NS-126, (S)-fluoromethyl 6,9-difluoro-17-[(2-furanylcarbonyl)oxy]-11-hydroxy-16-methyl-3-oxo-androsta-1,4-diene-17-carbothionate and (S)-(2-oxo-tetrahydro-furan-3S-yl) 6,9-difluoro-11-hydroxy-16-methyl-3-oxo-17-propionyloxy-androsta-1,4-diene-17-carbothionate, optionally in the form of the racemates, enantiomers or diastereomers thereof and optionally in the form of the salts and derivatives, solvates and/or hydrates thereof.

Particularly preferably the steroid is selected from among budesonide, fluticasone, mometasone, ciclesonide and (S)-fluoromethyl 6,9-difluoro-17-[(2-furanylcarbonyl)oxy]-11-hydroxy-16-methyl-3-oxo-androsta-1,4-diene-17-carbothionate, optionally in the form of the racemates, enantiomers or diastereomers thereof and optionally in the form of the salts and derivatives, solvates and/or hydrates thereof.

Any reference to steroids includes a reference to any salts or derivatives, hydrates or solvates thereof which may exist. Examples of possible salts and derivatives of the steroids may be: alkali metal salts, such as for example sodium or potassium salts, sulfobenzoates, phosphates, isonicotinates, acetates, propionates, dihydrogen phosphates, palmitates, pivalates or furoates thereof.

PDE4 inhibitors which may be used are preferably compounds selected from among enprofyllin, theophyllin, roflumilast, ariflo (cilomilast), tofimilast, pumafentrin, lirimilast, arofyllin, atizoram, D-4396 (Sch-351591), AWD-12-281 (GW-842470), NCS-613, CDP-840, D-4418, PD-168787, T-440, T-2585, V-11294A, CI-1018, CDC-801, CDC-3052, D-22888, YM-58997, Z-15370, N-(3,5-dichloro-1-oxo-pyridin-4-yl)-4-difluoromethoxy-3-cyclopropylmethoxybenzamide, (−)p-[(4aR*,10bS*)-9-ethoxy-1,2,3,4,4a,10b-hexahydro-8-methoxy-2-methylbenzo[s][1,6]naphthyridin-6-yl]-N,N-diisopropylbenzamide, (R)-(+)-1-(4-bromobenzyl)-4-[(3-cyclopentyloxy)-4-methoxyphenyl]-2-pyrrolidone, 3-(cyclopentyloxy-4-methoxyphenyl)-1-(4-N′-[N-2-cyano-5-methyl-isothioureido]benzyl)-2-pyrrolidone, cis[4-cyano-4-(3-cyclo-pentyloxy-4-methoxyphenyl)cyclohexane-1-carboxylic acid], 2-carbomethoxy-4-cyano-4-(3-cyclopropylmethoxy-4-difluoromethoxyphenyl)cyclohexane-1-one, cis[4-cyano-4-(3-cyclopropylmethoxy-4-difluoromethoxyphenyl)cyclohexan-1-ol], (R)-(+)-ethyl[4-(3-cyclopentyloxy-4-methoxyphenyl)pyrrolidin-2-ylidene]acetate, (S)-(−)-ethyl[4-(3-cyclopentyloxy-4-methoxyphenyl)pyrrolidin-2-ylidene]acetate, 9-cyclopentyl-5,6-dihydro-7-ethyl-3-(2-thienyl)-9H-pyrazolo[3,4-c]-1,2,4-triazolo[4,3-a]pyridine and 9-cyclopentyl-5,6-dihydro-7-ethyl-3-(tert-butyl)-9H-pyrazolo[3,4-c]-1,2,4-triazolo[4,3-a]pyridine, optionally in the form of the racemates, enantiomers or diastereomers and optionally in the form of the pharmacologically acceptable acid addition salts, solvates and/or hydrates thereof.

Particularly preferably the PDE4-inhibitor is selected from among roflumilast, ariflo (cilomilast), arofyllin, AWD-12-281 (GW-842470), 2-carbomethoxy-4-cyano-4-(3-cyclopropylmethoxy-4-difluoromethoxyphenyl)cyclohexan-1-one, cis[4-cyano-4-(3-cyclopropylmethoxy-4-difluoromethoxyphenyl)cyclohexan-1-ol], atizoram, Z-15370, 9-cyclopentyl-5,6-dihydro-7-ethyl-3-(2-thienyl)-9H-pyrazolo[3,4-c]-1,2,4-triazolo[4,3-a]pyridine and 9-cyclopentyl-5,6-dihydro-7-ethyl-3-(tert-butyl)-9H-pyrazolo[3,4-c]-1,2,4-triazolo[4,3-a]pyridine, optionally in the form of the racemates, enantiomers or diastereomers and optionally in the form of the pharmacologically acceptable acid addition salts, solvates and/or hydrates thereof.

By acid addition salts with pharmacologically acceptable acids which the above-mentioned PDE4-inhibitors might be in a position to form are meant, for example, salts selected from among the hydrochloride, hydrobromide, hydroiodide, hydrosulphate, hydrophosphate, hydromethanesulphonate, hydronitrate, hydromaleate, hydroacetate, hydrobenzoate, hydrocitrate, hydrofumarate, hydrotartrate, hydrooxalate, hydrosuccinate, hydrobenzoate and hydro-p-toluenesulphonate, preferably hydrochloride, hydrobromide, hydrosulphate, hydrophosphate, hydrofumarate and hydromethanesulphonate.

LTD4-antagonists which may be used are preferably compounds selected from among montelukast, pranlukast, zafirlukast, MCC-847 (ZD-3523), MN-001, MEN-91507 (LM-1507), VUF-5078, VUF-K-8707, L-733321, 1-(((R)-(3-(2-(6,7-difluoro-2-quinolinyl)ethenyl)phenyl)-3-(2-(2-hydroxy-2-propyl)phenyl)thio)-methylcyclopropane-acetic acid, 1-(((1(R)-3(3-(2-(2,3-dichlorothieno[3,2-b]pyridin-5-yl)-(E)-ethenyl)phenyl)-3-(2-(1-hydroxy-1-methylethyl)phenyl)-propyl)thio)methyl)cyclopropane-acetic acid and [2-[[2-(4-tert-butyl-2-thiazolyl)-5-benzofuranyl]oxymethyl]phenyl]acetic acid, optionally in the form of the racemates, enantiomers or diastereomers, optionally in the form of the pharmacologically acceptable acid addition salts and optionally in the form of the salts and derivatives, solvates and/or hydrates thereof.

Particularly preferably the LTD4-antagonist is selected from among montelukast, pranlukast, zafirlukast, MCC-847 (ZD-3523), MN-001 and MEN-91507 (LM-1507), optionally in the form of the racemates, enantiomers or diastereomers, optionally in the form of the pharmacologically acceptable acid addition salts and optionally in the form of the salts and derivatives, solvates and/or hydrates thereof.

By acid addition salts with pharmacologically acceptable acids which the LTD4-antagonists may be capable of forming are meant, for example, salts selected from among the hydrochloride, hydrobromide, hydroiodide, hydrosulphate, hydrophosphate, hydromethanesulphonate, hydronitrate, hydromaleate, hydroacetate, hydrobenzoate, hydrocitrate, hydrofumarate, hydrotartrate, hydrooxalate, hydrosuccinate, hydrobenzoate and hydro-p-toluenesulphonate, preferably hydrochloride, hydrobromide, hydrosulphate, hydrophosphate, hydrofumarate and hydromethanesulphonate. By salts or derivatives which the LTD4-antagonists may be capable of forming are meant, for example: alkali metal salts, such as, for example, sodium or potassium salts, alkaline earth metal salts, sulphobenzoates, phosphates, isonicotinates, acetates, propionates, dihydrogen phosphates, palmitates, pivalates or furoates.

The EGFR-inhibitors used are preferably compounds selected from among 4-[(3-chloro-4-fluorophenyl)amino]-6-{[4-(morpholin-4-yl)-1-oxo-2-buten-1-yl]amino}-7-cyclopropylmethoxy-quinazoline, 4-[(3-chloro-4-fluorophenyl)amino]-6-{[4-(N,N-diethylamino)-1-oxo-2-buten-1-yl]amino}-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopropylmethoxy-quinazoline, 4-[(R)-(1-phenyl-ethyl)amino]-6-{[4-(morpholin-4-yl)-1-oxo-2-buten-1-yl]amino}-7-cyclopentyloxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline, 4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline, 4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline, 4-[(3-chloro-4-fluoro-phenyl)amino]-6-[2-((S)-6-methyl-2-oxo-morpholin-4-yl)-ethoxy]-7-methoxy-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-cyclopentyloxy-quinazoline, 4-[(R)-(1-phenyl-ethyl)amino]-6-{[4-(N,N-bis-(2-methoxy-ethyl)-amino)-1-oxo-2-buten-1-yl]amino}-7-cyclopropylmethoxy-quinazoline, 4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline, 4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline, 4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline, 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline, 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline, 4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline, 4-[(3-chloro-4-fluorophenyl)amino]-6-{[4-(N-cyclopropyl-N-methyl-amino)-1-oxo-2-buten-1-yl]amino}-7-cyclopentyloxy-quinazoline, 4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline, 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline, 4-[(3-ethynyl-phenyl)amino]-6,7-bis-(2-methoxy-ethoxy)-quinazoline, 4-[(3-chloro-4-fluorophenyl)amino]-7-[3-(morpholin-4-yl)-propyloxy]-6-[(vinylcarbonyl)amino]-quinazoline, 4-[(R)-(1-phenyl-ethyl)amino]-6-(4-hydroxy-phenyl)-7H-pyrrolo[2,3-d]pyrimidine, 3-cyano-4-[(3-chloro-4-fluorophenyl)amino]-6-{[4-(N,N-dimethylamino)-1-oxo-2-buten-1-yl]amino}-7-ethoxy-quinoline, 4-{[3-chloro-4-(3-fluoro-benzyloxy)-phenyl]amino}-6-(5-{[(2-methanesulphonyl-ethyl)amino]methyl}-furan-2-yl)quinazoline, 4-[(R)-(1-phenyl-ethyl)amino]-6-{[4-((R)-6-methyl-2-oxo-morpholin-4-yl)-1-oxo-2-buten-1-yl]amino}-7-methoxy-quinazoline, 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline, 4-[(3-chloro-4-fluorophenyl)amino]-6-({4-[N,N-bis-(2-methoxy-ethyl)-amino]-1-oxo-2-buten-1-yl}amino)-7-[(tetrahydrofuran-2-yl)methoxy]-quinazoline, 4-[(3-ethynyl-phenyl)amino]-6-{[4-(5,5-dimethyl-2-oxo-morpholin-4-yl)-1-oxo-2-buten-1-yl]amino}-quinazoline, 4-[(3-chloro-4-fluoro-phenyl)amino]-6-[2-(2,2-dimethyl-6-oxo-morpholin-4-yl)-ethoxy]-7-methoxy-quinazoline, 4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline, 4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline, 4-[(3-chloro-4-fluoro-phenyl)amino]-6-{2-[4-(2-oxo-morpholin-4-yl)-piperidin-1-yl]ethoxy}-7-methoxy-quinazoline, 4-[(3-chloro-4-fluoro-phenyl)amino]-6-[1-(tert.-butyloxycarbonyl)-piperidin-4-yloxy]-7-methoxy-quinazoline, 4-[(3-chloro-4-fluoro-phenyl)amino]-6-(trans-4-amino-cyclohexan-1-yloxy)-7-methoxy-quinazoline, 4-[(3-chloro-4-fluoro-phenyl)amino]-6-(trans-4-methanesulphonylamino-cyclohexan-1-yloxy)-7-methoxy-quinazoline, 4-[(3-chloro-4-fluoro-phenyl)amino]-6-(tetrahydropyran-3-yloxy)-7-methoxy-quinazoline, 4-[(3-chloro-4-fluoro-phenyl)amino]-6-(1-methyl-piperidin-4-yloxy)-7-methoxy-quinazoline, 4-[(3-chloro-4-fluoro-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(methoxymethyl)carbonyl]-piperidin-4-yloxy}-7-methoxy-quinazoline, 4-[(3-chloro-4-fluoro-phenyl)amino]-6-(piperidin-3-yloxy)-7-methoxy-quinazoline, 4-[(3-chloro-4-fluoro-phenyl)amino]-6-[1-(2-acetylamino-ethyl)-piperidin-4-yloxy]-7-methoxy-quinazoline, 4-[(3-chloro-4-fluoro-phenyl)amino]-6-(tetrahydropyran-4-yloxy)-7-ethoxy-quinazoline, 4-[(3-chloro-4-fluoro-phenyl)amino]-6-((S)-tetrahydrofuran-3-yloxy)-7-hydroxy-quinazoline, 4-[(3-chloro-4-fluoro-phenyl)amino]-6-(tetrahydropyran-4-yloxy)-7-(2-methoxy-ethoxy)-quinazoline, 4-[(3-chloro-4-fluoro-phenyl)amino]-6-{trans-4-[(dimethylamino)sulphonylamino]cyclohexan-1-yloxy}-7-methoxy-quinazoline, 4-[(3-chloro-4-fluoro-phenyl)amino]-6-{trans-4-[(morpholin-4-yl)carbonylamino]-cyclohexan-1-yloxy}-7-methoxy-quinazoline, 4-[(3-chloro-4-fluoro-phenyl)amino]-6-{trans-4-[(morpholin-4-yl)sulphonylamino]-cyclohexan-1-yloxy}-7-methoxy-quinazoline, 4-[(3-chloro-4-fluoro-phenyl)amino]-6-(tetrahydropyran-4-yloxy)-7-(2-acetylamino-ethoxy)-quinazoline, 4-[(3-chloro-4-fluoro-phenyl)amino]-6-(tetrahydropyran-4-yloxy)-7-(2-methanesulphonylamino-ethoxy)-quinazoline, 4-[(3-chloro-4-fluoro-phenyl)amino]-6-{1-[(piperidin-1-yl)carbonyl]-piperidin-4-yloxy}-7-methoxy-quinazoline, 4-[(3-chloro-4-fluoro-phenyl)amino]-6-(1-aminocarbonylmethyl-piperidin-4-yloxy)-7-methoxy-quinazoline, 4-[(3-chloro-4-fluoro-phenyl)amino]-6-(cis-4-{N-[tetrahydropyran-4-yl)carbonyl]-N-methyl-amino}-cyclohexan-1-yloxy)-7-methoxy-quinazoline, 4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazolin; 4-{2-[4-(3-chloro-4-fluoro-phenylamino)-7-methoxy-quinazolin-6-yloxy]-ethyl}-6-methyl-morpholine-2-one, 4-{4-[4-(3-chloro-2-fluoro-phenylamino)-7-methoxy-quinazolin-6-yloxy]-cyclohexyl}-1-methyl-piperazin-2-one, 4-[(3-chloro-4-fluoro-phenyl)amino]-6-(cis-4-{N-[(morpholin-4-yl)sulphonyl]-N-methyl-amino}-cyclohexan-1-yloxy)-7-methoxy-quinazoline, 4-[(3-chloro-4-fluoro-phenyl)amino]-6-(trans-4-ethansulphonylamino-cyclohexan-1-yloxy)-7-methoxy-quinazoline, 4-[(3-chloro-4-fluoro-phenyl)amino]-6-(1-methanesulphonyl-piperidin-4-yloxy)-7-ethoxy-quinazoline, 4-[(3-chloro-4-fluoro-phenyl)amino]-6-(1-methanesulphonyl-piperidin-4-yloxy)-7-(2-methoxy-ethoxy)-quinazoline, 4-[(3-chloro-4-fluoro-phenyl)amino]-6-[1-(2-methoxy-acetyl)-piperidin-4-yloxy]-7-(2-methoxy-ethoxy)-quinazoline, 4-[(3-chloro-4-fluoro-phenyl)amino]-6-(cis-4-acetylamino-cyclohexan-1-yloxy)-7-methoxy-quinazoline, 4-[(3-ethynyl-phenyl)amino]-6-[1-(tert.-butyloxycarbonyl)-piperidin-4-yloxy]-7-methoxy-quinazoline, 4-[(3-ethynyl-phenyl)amino]-6-(tetrahydropyran-4-yloxy]-7-methoxy-quinazoline, 4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline, 4-[(3-chloro-4-fluoro-phenyl)amino]-6-(cis-4-{N-[(4-methyl-piperazin-1-yl)carbonyl]-N-methyl-amino}-cyclohexan-1-yloxy)-7-methoxy-quinazoline, 4-[(3-chloro-4-fluoro-phenyl)amino]-6-{cis-4-[(morpholin-4-yl)carbonylamino]-cyclohexan-1-yloxy}-7-methoxy-quinazoline, 4-[(3-chloro-4-fluoro-phenyl)amino]-6-{1-[2-(2-oxopyrrolidin-1-yl)ethyl]-piperidin-4-yloxy}-7-methoxy-quinazoline, 4-[(3-chloro-4-fluoro-phenyl)amino]-6-{1-[(morpholin-4-yl)carbonyl]-piperidin-4-yloxy}-7-(2-methoxy-ethoxy)-quinazoline, 4-[(3-ethynyl-phenyl)amino]-6-(1-acetyl-piperidin-4-yloxy)-7-methoxy-quinazoline, 4-[(3-ethynyl-phenyl)amino]-6-(1-methyl-piperidin-4-yloxy)-7-methoxy-quinazoline, 4-[(3-ethynyl-phenyl)amino]-6-(1-methanesulphonyl-piperidin-4-yloxy)-7-methoxy-quinazoline, 4-[(3-chloro-4-fluoro-phenyl)amino]-6-(1-methyl-piperidin-4-yloxy)-7(2-methoxy-ethoxy)-quinazoline, 4-[(3-chloro-4-fluoro-phenyl)amino]-6-(1-isopropyloxycarbonyl-piperidin-4-yloxy)-7-methoxy-quinazoline, 4-[(3-chloro-4-fluoro-phenyl)amino]-6-(cis-4-methylamino-cyclohexan-1-yloxy)-7-methoxy-quinazoline, 4-[(3-chloro-4-fluoro-phenyl)amino]-6-{cis-4-[N-(2-methoxy-acetyl)-N-methyl-amino]-cyclohexan-1-yloxy}-7-methoxy-quinazoline, 4-[(3-ethynyl-phenyl)amino]-6-(piperidin-4-yloxy)-7-methoxy-quinazoline, 4-[(3-ethynyl-phenyl)amino]-6-[1-(2-methoxy-acetyl)-piperidin-4-yloxy]-7-methoxy-quinazoline, 4-[(3-ethynyl-phenyl)amino]-6-{1-[(morpholin-4-yl)carbonyl]-piperidin-4-yloxy}-7-methoxy-quinazoline, 4-[(3-chloro-4-fluoro-phenyl)amino]-6-{1-[(cis-2,6-dimethyl-morpholin-4-yl)carbonyl]-piperidin-4-yloxy}-7-methoxy-quinazoline, 4-[(3-chloro-4-fluoro-phenyl)amino]-6-{1-[(2-methyl-morpholin-4-yl)carbonyl]-piperidin-4-yloxy}-7-methoxy-quinazoline, 4-[(3-chloro-4-fluoro-phenyl)amino]-6-{1-[(S,S)-(2-oxa-5-aza-bicyclo[2,2,1]hept-5-yl)carbonyl]-piperidin-4-yloxy}-7-methoxy-quinazoline, 4-[(3-chloro-4-fluoro-phenyl)amino]-6-{1-[(N-methyl-N-2-methoxyethyl-amino)carbonyl]-piperidin-4-yloxy}-7-methoxy-quinazoline, 4-[(3-chloro-4-fluoro-phenyl)amino]-6-(1-ethyl-piperidin-4-yloxy)-7-methoxy-quinazoline, 4-[(3-chloro-4-fluoro-phenyl)amino]-6-{1-[(2-methoxyethyl)carbonyl]-piperidin-4-yloxy}-7-methoxy-quinazoline, 4-[(3-chloro-4-fluoro-phenyl)amino]-6-{1-[(3-methoxypropyl-amino)-carbonyl]-piperidin-4-yloxy}-7-methoxy-quinazoline, 4-[(3-chloro-4-fluoro-phenyl)amino]-6-[cis-4-(N-methanesulphonyl-N-methyl-amino)-cyclohexan-1-yloxy]-7-methoxy-quinazoline, 4-[(3-chloro-4-fluoro-phenyl)amino]-6-[cis-4-(N-acetyl-N-methyl-amino)-cyclohexan-1-yloxy]-7-methoxy-quinazoline, 4-[(3-chloro-4-fluoro-phenyl)amino]-6-(trans-4-methylamino-cyclohexan-1-yloxy)-7-methoxy-quinazoline, 4-[(3-chloro-4-fluoro-phenyl)amino]-6-[trans-4-(N-methanesulphonyl-N-methyl-amino)-cyclohexan-1-yloxy]-7-methoxy-quinazoline, 4-[(3-chloro-4-fluoro-phenyl)amino]-6-(trans-4-dimethylamino-cyclohexan-1-yloxy)-7-methoxy-quinazoline, 4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline, 4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline, 4-[(3-chloro-4-fluoro-phenyl)amino]-6-(1-methanesulphonyl-piperidin-4-yloxy)-7-methoxy-quinazoline, 4-[(3-chloro-4-fluoro-phenyl)amino]-6-(1-cyano-piperidin-4-yloxy)-7-methoxy-quinazoline, [44(3-chloro-4-fluoro-phenyl)amino]-6-{[4-(homomorpholin-4-yl)-1-oxo-2-buten-1-yl]amino}-7-[(S)-(tetrahydrofuran-3-yl)oxy]-quinazoline, 4-[(3-chloro-4-fluoro-phenyl)amino]-7-(2-{4-[(S)-(2-oxo-tetrahydrofuran-5-yl)carbonyl]-piperazin-1-yl}-ethoxy)-6-[(vinylcarbonyl)amino]-quinazoline, 4-[(3-chloro-4-fluoro-phenyl)amino]-7-[2-((S)-6-methyl-2-oxo-morpholin-4-yl)-ethoxy]-6-[(vinylcarbonyl)amino]-quinazoline, 4-[(3-chloro-4-fluoro-phenyl)amino]-7-[4-((R)-6-methyl-2-oxo-morpholin-4-yl)-butyloxy]-6-[(vinylcarbonyl)amino]-quinazoline, 4-[(3-chloro-4-fluoro-phenyl)amino]-7-[4-((S)-6-methyl-2-oxo-morpholin-4-yl)-butyloxy]-6-[(vinylcarbonyl)amino]-quinazoline, 4-[(3-chloro-4-fluoro-phenyl)amino]-7-(2-{4-[(S)-(2-oxo-tetrahydrofuran-5-yl)carbonyl]-piperazin-1-yl}-ethoxy)-6-[(vinylcarbonyl)amino]-quinazoline, 4-[(3-chloro-4-fluoro-phenyl)amino]-7-[2-((S)-6-methyl-2-oxo-morpholin-4-yl)-ethoxy]-6-[(vinylcarbonyl)amino]-quinazoline, 4-[(3-chloro-4-fluoro-phenyl)amino]-7-[4-((R)-6-methyl-2-oxo-morpholin-4-yl)-butyloxy]-6-[(vinylcarbonyl)amino]-quinazoline, 4-[(3-chloro-4-fluoro-phenyl)amino]-7-[4-((S)-6-methyl-2-oxo-morpholin-4-yl)-butyloxy]-6-[(vinylcarbonyl)amino]quinazoline, cetuximab, trastuzumab, ABX-EGF, Mab ICR-62, gefitinib, canertinib and erlotinib, optionally in the form of the racemates, enantiomers or diastereomers thereof, optionally in the form of the pharmacologically acceptable acid addition salts thereof, the solvates and/or hydrates thereof.

By acid addition salts with pharmacologically acceptable acids which the EGFR-inhibitors may be capable of forming are meant, for example, salts selected from among the hydrochloride, hydrobromide, hydroiodide, hydrosulphate, hydrophosphate, hydromethanesulphonate, hydronitrate, hydromaleate, hydroacetate, hydrobenzoate, hydrocitrate, hydrofumarate, hydrotartrate, hydrooxalate, hydrosuccinate, hydrobenzoate and hydro-p-toluenesulphonate, preferably hydrochloride, hydrobromide, hydrosulphate, hydrophosphate, hydrofumarate and hydromethanesulphonate.

Examples of dopamine agonists which may be used preferably include compounds selected from among bromocriptine, cabergoline, alpha-dihydroergocryptine, lisuride, pergolide, pramipexol, roxindol, ropinirol, talipexol, terguride and viozan. Any reference to the above-mentioned dopamine agonists within the scope of the present invention includes a reference to any pharmacologically acceptable acid addition salts and optionally hydrates thereof which may exist. By the physiologically acceptable acid addition salts which may be formed by the above-mentioned dopamine agonists are meant, for example, pharmaceutically acceptable salts which are selected from the salts of hydrochloric acid, hydrobromic acid, sulphuric acid, phosphoric acid, methanesulphonic acid, acetic acid, fumaric acid, succinic acid, lactic acid, citric acid, tartaric acid and maleic acid.

Examples of H1-antihistamines preferably include compounds selected from among epinastine, cetirizine, azelastine, fexofenadine, levocabastine, loratadine, mizolastine, ketotifen, emedastine, dimetinden, clemastine, bamipin, cexchlorpheniramine, pheniramine, doxylamine, chlorophenoxamine, dimenhydrinate, diphenhydramine, promethazine, ebastine, desloratidine and meclozine. Any reference to the above-mentioned H1-antihistamines within the scope of the present invention includes a reference to any pharmacologically acceptable acid addition salts which may exist.

Examples of PAF-antagonists preferably include compounds selected from among 4-(2-chlorophenyl)-9-methyl-2-[3(4-morpholinyl)-3-propanon-1-yl]-6H-thieno-[3,2-f]-[1,2,4]triazolo[4,3-a][1,4]diazepines, 6-(2-chlorophenyl)-8,9-dihydro-1-methyl-8-[(4-morpholinyhcarbonyl]-4H,7H-cyclo-penta-[4,5]thieno-[3,2-f][1,2,4]triazolo[4,3-a][1,4]diazepines. Any reference to the above-mentioned above-mentioned PAF-antagonists includes within the scope of the present invention a reference to any pharmacologically acceptable acid addition salts thereof which may exist.

MRP4-inhibitors used are preferably compounds selected from among N-acetyl-dinitrophenyl-cysteine, cGMP, cholate, diclofenac, dehydroepiandrosterone 3-glucuronide, dehydroepiandrosterone 3-sulphate, dilazep, dinitrophenyl-s-glutathione, estradiol 17-beta-glucuronide, estradiol 3,17-disulphate, estradiol 3-glucuronide, estradiol 3-sulphate, estrone 3-sulphate, flurbiprofen, folate, N5-formyl-tetrahydrofolate, glycocholate, clycolithocholic acid sulphate, ibuprofen, indomethacin, indoprofen, ketoprofen, lithocholic acid sulphate, methotrexate, MK571 ((E)-3-[[[3-[2-(7-chloro-2-quinolinyl)ethenyl]phenyl]-[[3-dimethylamino)-3-oxopropyl]thio]methyl]thio]-propanoic acid), alpha-naphthyl-beta-D-glucuronide, nitrobenzyl mercaptopurine riboside, probenecid, PSC833, sildenafil, sulfinpyrazone, taurochenodeoxycholate, taurocholate, taurodeoxycholate, taurolithocholate, taurolithocholic acid sulphate, topotecan, trequinsin and zaprinast, dipyridamole, optionally in the form of the racemates, enantiomers, diastereomers and the pharmacologically acceptable acid addition salts and hydrates thereof.

The invention relates more preferably to the use of MRP4-inhibitors for preparing a pharmaceutical composition for treating respiratory complaints, containing the SYK-inhibitors and MRP4-inhibitors according to the invention, the MRP4-inhibitors preferably being selected from among dehydroepiandrosterone 3-sulphate, estradiol 3,17-disulphate, flurbiprofen, indomethacin, indoprofen, MK571, taurocholate, optionally in the form of the racemates, enantiomers, diastereomers and the pharmacologically acceptable acid addition salts and hydrates thereof. The separation of enantiomers from the racemates can be carried out using methods known from the art (e.g. chromatography on chiral phases, etc.).

By acid addition salts with pharmacologically acceptable acids are meant, for example, salts selected from among the hydrochlorides, hydrobromides, hydroiodides, hydrosulphates, hydrophosphates, hydromethanesulphonates, hydronitrates, hydromaleates, hydroacetates, hydrobenzoates, hydrocitrates, hydrofumarates, hydrotartrates, hydrooxalates, hydrosuccinates, hydrobenzoates and hydro-p-toluenesulphonates, preferably the hydrochlorides, hydrobromides, hydrosulphates, hydrophosphates, hydrofumarates and hydromethanesulphonates.

The invention further relates to pharmaceutical preparations which contain a triple combination of the SYK-inhibitors, MRP4-inhibitors and another active substance according to the invention, such as, for example, an anticholinergic, a PDE4 inhibitor, a steroid, an LTD4-antagonist or a betamimetic, and the preparation thereof and the use thereof for treating respiratory complaints.

Compounds which may be used as iNOS inhibitors are compounds selected from among: S-(2-aminoethyl)isothiourea, aminoguanidine, 2-aminomethylpyridine, AMT, L-canavanine, 2-iminopiperidine, S-isopropylisothiourea, S-methylisothiourea, S-ethylisothiourea, S-methyltiocitrullin, S-ethylthiocitrulline, L-NA (N^{ω}-nitro-L-arginine), L-NAME (N^{ω}-nitro-L-argininemethylester), L-NMMA (N^{G}-monomethyl-L-arginine), L-NIO (N^{ω}-iminoethyl-L-ornithine), L-NIL (N^{ω}-iminoethyl-lysine), (S)-6-acetim idoylamino-2-amino-hexanoic acid (1H-tetrazol-5-yl)-amide (SC-51) (J. Med. Chem. 2002, 45, 1686-1689), 1400W, (S)-4-(2-acetimidoylamino-ethylsulphanyl)-2-amino-butyric acid (GW274150) (Bioorg. Med. Chem. Lett. 2000, 10, 597-600), 2-[2-(4-methoxy-pyridin-2-yl)-ethyl]-3H-imidazo[4,5-b]pyridine (BYK191023) (Mol. Pharmacol. 2006, 69, 328-337), 2-((R)-3-amino-1-phenyl-propoxy)-4-chloro-5-fluorobenzonitrile (WO 01/62704), 2-((1R,3S)-3-amino-4-hydroxy-1-thiazol-5-yl-butylsulphanyl)-6-trifluoromethyl-nicotinonitrile (WO 2004/041794), 2-((1R,3S)-3-amino-4-hydroxy-1-thiazol-5-yl-butylsulphanyl)-4-chloro-benzonitrile (WO 2004/041794), 2-((1R,3S)-3-amino-4-hydroxy-1-thiazol-5-yl-butylsulphanyl)-5-chloro-benzonitrile (WO 2004/041794), (2S,4R)-2-amino-4-(2-chloro-5-trifluoromethyl-phenylsulphanyl)-4-thiazol-5-yl-butan-1-ol (WO 2004/041794), 2-((1R,3S)-3-amino-4-hydroxy-1-thiazol-5-yl-butylsulphanyl)-5-chloro-nicotinonitrile (WO 2004/041794), 4-((S)-3-amino-4-hydroxy-1-phenyl-butylsulphanyl)-6-methoxy-nicotinonitrile (WO 02/090332), substituted 3-phenyl-3,4-dihydro-1-isoquinolinamine such as e.g. AR-C102222 (J. Med. Chem. 2003, 46, 913-916), (1S,5S,6R)-7-chloro-5-methyl-2-aza-bicyclo[4.1.0]hept-2-en-3-ylamine (ONO-1714) (Biochem. Biophys. Res. Commun. 2000, 270, 663-667), (4R,5R)-5-ethyl-4-methyl-thiazolidin-2-ylideneamine (Bioorg. Med. Chem. 2004, 12, 4101), (4R,5R)-5-ethyl-4-methyl-selenazolidin-2-ylideneamine (Bioorg. Med. Chem. Lett. 2005, 15, 1361), 4-aminotetrahydrobiopterine (Curr. Drug Metabol. 2002, 3, 119-121), (E)-3-(4-chlorophenyl)-N-(1-{2-oxo-2-[4-(6-trifluoromethyl-pyrimidin-4-yloxy)-piperidin-1-yl]-ethylcarbamoyl}-2-pyridin-2-yl-ethyl)-acrylamide (FR260330) (Eur. J. Pharmacol. 2005, 509, 71-76), 3-(2,4-difluoro-phenyl)-6-[2-(4-imidazol-1-ylmethyl-phenoxy)-ethoxy]-2-phenyl-pyridine (PPA250) (J. Pharmacol. Exp. Ther. 2002, 303, 52-57), methyl 3-{[(benzo[1,3]dioxol-5-ylmethyl)-carbamoyl]-methyl}-4-(2-imidazol-1-yl-pyrimidin-4-yl)-piperazine-1-carboxylate (BBS-1) (Drugs Future 2004, 29, 45-52), (R)-1-(2-imidazol-1-yl-6-methyl-pyrimidin-4-yl)-pyrrolidine-2-carboxylic acid (2-benzo[1,3]dioxol-5-yl-ethyl)-amide (BBS-2) (Drugs Future 2004, 29, 45-52) and the pharmaceutical salts, prodrugs or solvates thereof.

Examples of iNOS-inhibitors within the scope of the present invention may also include antisense oligonucleotides, particularly those antisense oligonucleotides which bind iNOS-coding nucleic acids. For example, WO 01/52902 describes antisense oligonucleotides, particularly antisense oligonucleotides, which bind iNOS coding nucleic acids, for modulating the expression of iNOS. iNOS-antisense oligonucleotides as described particularly in WO 01/52902 may therefore also be combined with the PDE4-inhibitors of the present invention on account of their similar effect to the iNOS-inhibitors.

### 8. FORMULATIONS

Suitable forms for administration are for example tablets, capsules, solutions, syrups, emulsions or inhalable powders or aerosols. The content of the pharmaceutically effective compound(s) in each case should be in the range from 0.1 to 90 wt. %, preferably 0.5 to 50 wt. % of the total composition, i.e. in amounts which are sufficient to achieve the dosage range specified hereinafter.

The preparations may be administered orally in the form of a tablet, as a powder, as a powder in a capsule (e.g. a hard gelatine capsule), as a solution or suspension. When administered by inhalation the active substance combination may be given as a powder, as an aqueous or aqueous-ethanolic solution or using a propellant gas formulation.

Preferably, therefore, pharmaceutical formulations are characterised by the content of one or more compounds of formula 1 according to the preferred embodiments above.

It is particularly preferable if the compounds of formula 1 are administered orally, and it is also particularly preferable if they are administered once or twice a day. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Capsules containing one or more active substances or combinations of active substances may for example be prepared by mixing the active substances with inert carriers such as lactose or sorbitol and packing them into gelatine capsules. Suitable suppositories may be made for example by mixing with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral administration the tablets may, of course, contain, apart from the abovementioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

It is also preferred if the compounds of formula 1 are administered by inhalation, particularly preferably if they are administered once or twice a day. For this purpose, the compounds of formula 1 have to be made available in forms suitable for inhalation. Inhalable preparations include inhalable powders, propellant-containing metered-dose aerosols or propellant-free inhalable solutions, which are optionally present in admixture with conventional physiologically acceptable excipients.

Within the scope of the present invention, the term propellant-free inhalable solutions also includes concentrates or sterile ready-to-use inhalable solutions. The preparations which may be used according to the invention are described in more detail in the next part of the specification.

### Inhalable Powders

If the active substances of formula 1 are present in admixture with physiologically acceptable excipients, the following physiologically acceptable excipients may be used to prepare the inhalable powders according to the invention: monosaccharides (e.g. glucose or arabinose), disaccharides (e.g. lactose, saccharose, maltose), oligo- and polysaccharides (e.g. dextran), polyalcohols (e.g. sorbitol, mannitol, xylitol), salts (e.g. sodium chloride, calcium carbonate) or mixtures of these excipients with one another. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred. Methods of preparing the inhalable powders according to the invention by grinding and micronising and by finally mixing the components together are known from the prior art.

### Propellant-Containing Inhalable Aerosols

The propellant-containing inhalable aerosols which may be used according to the invention may contain the compounds of formula 1 dissolved in the propellant gas or in dispersed form. The propellant gases which may be used to prepare the inhalation aerosols according to the invention are known from the prior art. Suitable propellant gases are selected from among hydrocarbons such as n-propane, n-butane or isobutane and halohydrocarbons such as preferably fluorinated derivatives of methane, ethane, propane, butane, cyclopropane or cyclobutane. The propellant gases mentioned above may be used on their own or in mixtures thereof. Particularly preferred propellant gases are fluorinated alkane derivatives selected from TG134a (1,1,1,2-tetrafluoroethane), TG227 (1,1,1,2,3,3,3-heptafluoropropane) and mixtures thereof. The propellant-driven inhalation aerosols used within the scope of the use according to the invention may also contain other ingredients such as co-solvents, stabilisers, surfactants, antioxidants, lubricants and pH adjusters. All these ingredients are known in the art.

### Propellant-Free Inhalable Solutions

The compounds of formula 1 according to the invention are preferably used to prepare propellant-free inhalable solutions and inhalable suspensions. Solvents used for this purpose include aqueous or alcoholic, preferably ethanolic solutions. The solvent may be water on its own or a mixture of water and ethanol. The solutions or suspensions are adjusted to a pH of 2 to 7, preferably 2 to 5, using suitable acids. The pH may be adjusted using acids selected from inorganic or organic acids. Examples of particularly suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. Preferred inorganic acids are hydrochloric and sulphuric acids. It is also possible to use the acids which have already formed an acid addition salt with one of the active substances. Of the organic acids, ascorbic acid, fumaric acid and citric acid are preferred. If desired, mixtures of the above acids may also be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

Co-solvents and/or other excipients may be added to the propellant-free inhalable solutions used for the purpose according to the invention. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include pharmacologically acceptable salts such as sodium chloride as isotonic agents. The preferred excipients include antioxidants such as ascorbic acid, for example, provided that it has not already been used to adjust the pH, vitamin A, vitamin E, tocopherols and similar vitamins or provitamins occurring in the human body. Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art.

For the treatment forms described above, ready-to-use packs of a medicament for the treatment of respiratory complaints are provided, containing an enclosed description including for example the words respiratory disease, COPD or asthma, together with a naphthyridine according to formula 1 and one or more combination partners selected from those described above.

1. A compounds of formula 1 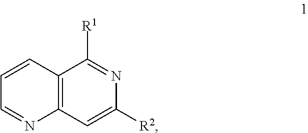
wherein:
R¹ is a group A selected from —O—R³, —NR³R⁴, —CR³R⁴R⁵, -(ethyne)-R³, —S—R³, —SO—R³, and —SO₂—R³, or
R¹ is a group B selected from
C₆₋₁₀-aryl,
a five- to ten-membered, mono- or bicyclic heteroaryl with 1-3 heteroatoms independently selected from N, O and S, wherein the heteroaryl is linked to the structure according to formula 1 via either a C atom or an N atom,
a three- to ten-membered, mono- or bicyclic, saturated or partially saturated heterocyclic group with 1-3 heteroatoms independently selected from N, O and S, wherein the heterocyclic group is linked to the structure according to formula 1 via either a C atom or an N atom, and
a 5- to 11-membered spiro group optionally containing 1, 2, or 3 heteroatoms independently selected from N, O and S, wherein the spiro group is linked to the structure according to formula 1 via either a C atom or an N atom,
wherein group B is optionally substituted by one or more groups independently selected from H, halogen, —C₁₋₃-alkyl, —NH(C₁₋₄-alkyl), —N(C₁₋₄-alkyl)₂, —NH₂, —C₁₋₃-alkyl-OH, —OH, oxo, —CO—NH₂, —C₁₋₃-alkylene-CO—NH₂, —CO—NH—(C₁₋₃-alkyl), —C₁₋₃-alkylene-CO—NH(C₁₋₃-alkyl), —CO—NH(C₃₋₅-cycloalkyl), —C₁₋₃-alkylene-CO—NH(C₃₋₅-cycloalkyl), —NH—CO—NH₂, —NH—CO—NH(C₁₋₃-alkyl), —NH—CO—N(C₁₋₃-alkyl)₂, O—C₁₋₃-alkyl, —(C₁₋₃-alkylene)-NH₂, -phenyl, and —CO—(C₁₋₅-alkyl), and
R² is 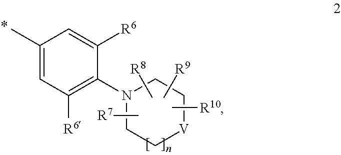
wherein:
V is CH₂, O, NH, S, SO, SO₂, N—(C₁₋₃-alkyl), N—(C₁₋₃-alkylene)-(C₃₋₇-cycloalkyl), N—(C₃₋₇-cycloalkyl), N—CO—C₁₋₆-alkyl, N—CO—(C₃₋₇-cycloalkyl), or N—(C₁₋₃-alkylene)-phenyl
n is 0-2
R⁶ and R^{6′} are independently selected from H, halogen, methyl, —O-methyl, ethyl, —O-ethyl, propyl, —O-propyl, OH, ═O, —CO—NH₂, —CO—NH—C₁₋₃-alkyl, —COOH, and —COO—C₁₋₃-alkyl
R⁷, R⁸, R⁹, and R¹⁰ are each independently H, C₁₋₃-alkyl, —O—(C₁₋₃-alkyl), F, ═O, or OH,
R³ is H or a group selected from C₁₋₆-alkyl, —C₁₋₆-fluoroalkyl, —(C₁₋₅-alkyl)-OH, —C₆₋₁₀-aryl, —C₁₋₄-alkylene-C₆₋₁₀-aryl, -ethenyl, —C₁₋₄-alkylene-(ethene), -ethynyl, —C₁₋₄-alkylene-(ethyne), —C₁₋₄-alkylene-(ethyne)-NH₂, —C₁₋₄-alkylene-(ethyne)-(C₁₋₄-alkylene)-NH₂, —CHOH—(C₁₋₄-alkylene)-NH₂, —(C₁₋₄-alkylene)-CHOH—(C₁₋₄-alkylene)-NH₂, —CHOH—NH₂, —(C₁₋₄-alkylene)-CHOH—NH₂, —NH(C₁₋₃-alkylene), —(C₁₋₄-alkylene)-NH(C₁₋₃-alkyl), mono- or bicyclic, saturated or partially saturated —C₃₋₁₀-cycloalkyl, mono- or bicyclic, saturated or partially saturated —(C₁₋₄-alkylene)-C₃₋₁₀-cycloalkyl, -(het), —(C₁₋₄-alkylene)-(het), -(hetaryl), and —(C₁₋₄-alkylene)-(hetaryl), wherein this group is optionally substituted by one or more groups independently selected from H, —OH, -oxo, —COOH, -halogen, —C₁₋₃-alkyl, —C₁₋₃-haloalkyl, —C₁₋₃-alkyl-OH, —C₃₋₇-cycloalkyl, —O—(C₁₋₄-alkyl), —NH(C₁₋₄-alkyl), —(C₁₋₄-alkylene)-NH(C₁₋₄-alkyl), —N(C₁₋₄-alkyl)₂, —(C₁₋₄-alkylene)-N(C₁₋₄-alkyl)₂, —NH—CO—NH₂, —(C₁₋₄-alkylene)-NH—CO—NH₂, —CO—NH₂, —(C₁₋₄-alkylene)-CO—NH₂, —CO—NH(C₁₋₃-alkyl), —(C₁₋₄-alkylene)-CO—NH(C₁₋₃-alkyl), —CO—N(C₁₋₃-alkyl)₂, —(C₁₋₄-alkylene)-CO—N(C₁₋₃-alkyl)₂, —NH—(CO)ₘ—NH₂, —NH—(C₁₋₄-alkylene)-(CO)ₘ—NH₂, —NH—(CO)ₘ—NH(C₁₋₃-alkyl), —NH—(C₁₋₄-alkylene)-(CO)ₘ—NH(C₁₋₃-alkyl), —NH—(CO)ₘ—N(C₁₋₃-alkyl)₂, —NH—(C₁₋₄-alkylene)-(CO)ₘ—N(C₁₋₃-alkyl)₂, —O—(C₂₋₄-alkylene)-NH₂, —O—(C₂₋₄-alkylene)-NH(C₁₋₃-alkyl), —O—(C₂₋₄-alkylene)-N(C₁₋₃-alkyl)₂, —NH—CO—(C₁₋₃-alkyl), —(C₁₋₄-alkylene)-NH—CO—(C₁₋₃-alkyl), —C₃₋₅-cycloalkyl, —SO₂—(C₁₋₄-alkyl), —SO₂—(C₃₋₅-cycloalkyl), —SO₂—NH₂, —SO₂—NH—C₁₋₃-alkyl, —SO₂—N(C₁₋₃-alkyl)₂, —SO₂-(het), —O-(het), —O—(C₁₋₄-alkylene)-(het), —NH-(het), —NH—(C₁₋₄-alkylene)-(het), —NH-(hetaryl), —NH—(C₁₋₄-alkylene)-(hetaryl), -(het), and —(C₁₋₄-alkylene)-(het),
wherein (het) is a three- to ten-membered, saturated or partially saturated, mono- or bicyclic, heterocyclic group optionally substituted by 1-3 groups selected from C₁₋₃-alkyl, halogen, CH₂—NH₂, NH₂, OH, CO—NH₂, and oxo, which contains 1-3 heteroatoms independently selected from N, O, and S, and
wherein (hetaryl) is a five- to ten-membered, mono- or bicyclic, heteroaryl optionally substituted by with 1-3 groups selected from C₁₋₃-alkyl, halogen, CH₂—NH₂, NH₂, OH, CO—NH₂, and oxo, which contains 1-3 heteroatoms independently selected from N, O, and S,
m is 0 or 1
R⁴ and R⁵ are each independently H, methyl, or ethyl,
or a pharmaceutically acceptable salts thereof.

2. The compounds of formula 1 according to claim 1, wherein n is 1, or a pharmaceutically acceptable salts thereof.

3. The compounds of formula 1 according to claim 1, wherein R⁶ and R^{6′} are each independently H, methyl, or —OCH₃, or a pharmaceutically acceptable salts thereof.

4. The compounds of formula 1 according to claim 1, wherein R⁷, R⁸, R⁹, and R¹⁰ are each independently H or —OCH₃, or a pharmaceutically acceptable salts thereof.

5. The compound of formula 1 according to claim 1, wherein V is N—CH₃, O, or N—(C₁₋₃-alkylene)-phenyl, and or a pharmaceutically acceptable salts thereof.

6. The compounds of formula 1 according to claim 1, wherein R¹ is —O—R³, —NR³R⁴, —CR³R⁴R⁵, or -(ethyne)-R³, or a pharmaceutically acceptable salts thereof.

7. The compounds of formula 1 according to claim 1, wherein:
R¹ is NR³R⁴,
R⁴ is H, and
R³ is C₁₋₆-alkyl, C₆₋₁₀ aryl, —C₁₋₄-alkylene-C₆₋₁₀-aryl, -(het), —(C₁₋₄-alkylene)-(het), -(hetaryl), or —(C₁₋₄-alkylene)-(hetaryl), wherein R³ is optionally substituted by one or more groups independently selected from H, OH, -oxo, —COOH, —C₁₋₃-alkyl, —C₁₋₃-haloalkyl, —C₁₋₃-alkyl-OH, —CO—NH₂, —(C₁₋₄-alkylene)-CO—NH₂, —NH—SO₂—CH₃, —CO—NH(C₁₋₃-alkyl), —(C₁₋₄-alkylene)-CO—NH(C₁₋₃-alkyl), —CO—N(C₁₋₃-alkyl)₂, —(C₁₋₄-alkylene)-CO—N(C₁₋₃-alkyl)₂, —NH—(CO)ₘ—NH₂, —NH—(C₁₋₄-alkylene)-(CO)ₘ—NH₂, —NH—(CO)ₘ—NH(C₁₋₃-alkyl), —NH—(C₁₋₄-alkylene)-(CO)ₘ—NH(C₁₋₃-alkyl), —NH—(CO)ₘ—N(C₁₋₃-alkyl)₂, and —NH—(C₁₋₄-alkylene)-(CO)ₘ—N(C₁₋₃-alkyl)₂,
or a pharmaceutically acceptable salts thereof.

8. The compounds of formula 1 according to claim 7, wherein:
R¹ is —NR³R⁴,
R⁴ is denotes H, and
R³ is —C₆₋₁₀-aryl, —C₁₋₄-alkylene-C₆₋₁₀-aryl, -(het), —(C₁₋₄-alkylene)-(het), -(hetaryl), or —(C₁₋₄-alkylene)-(hetaryl), wherein R³ is optionally substituted by one or more groups independently selected from H, —OH, -oxo, —COOH, —C₁₋₃-alkyl, —CO—NH₂, —(C₁₋₄-alkylene)-CO—NH₂, —CO—NH(C₁₋₃-alkyl), —(C₁₋₄-alkylene)-CO—NH(C₁₋₃-alkyl), —CO—N(C₁₋₃-alkyl)₂, and —(C₁₋₄-alkylene)-CO—N(C₁₋₃-alkyl)₂, or a pharmaceutically acceptable salt thereof.

9. The compounds of formula 1 according to claim 1, wherein:
R¹ is —OR³,
R⁴ is H, and
R³ is —C₆₋₁₀-aryl, —C₁₋₄-alkylene-C₆₋₁₀-aryl, -(het), —(C₁₋₄-alkylene)-(het), -(hetaryl), or —(C₁₋₄-alkylene)-(hetaryl), wherein R³ is optionally substituted by one or more groups independently selected from H, —OH, -oxo, —COOH, —C₁₋₃-alkyl, —C₁₋₃-haloalkyl, —C₁₋₃-alkyl-OH, —CO—NH₂, —(C₁₋₄-alkylene)-CO—NH₂, —CO—NH(C₁₋₃-alkyl), —(C₁₋₄-alkylene)-CO—NH(C₁₋₃-alkyl), —CO—N(C₁₋₃-alkyl)₂, —(C₁₋₄-alkylene)-CO—N(C₁₋₃-alkyl)₂, —NH—(CO)ₘ—NH₂, —NH—(C₁₋₄-alkylene)-(CO)ₘ—NH₂, —NH(CO)ₘ—NH(C₁₋₃-alkyl), —NH(C₁₋₄-alkylene)-(CO)ₘ—NH(C₁₋₃-alkyl), —NH—(CO)ₘ—N(C₁₋₃-alkyl)₂, and —NH—(C₁₋₄-alkylene)-(CO)ₘ—N(C₁₋₃-alkyl)₂, or a pharmaceutically acceptable salt thereof.

10. The compounds of formula 1 according to claim 1, wherein:
R¹ is —CR³R⁴R⁵,
R⁴ is H, or methyl,
R⁵ is H, or methyl, and
R³ is —C₆₋₁₀-aryl, —C₁₋₄-alkylene-C₆₋₁₀-aryl, -(het), —(C₁₋₄-alkylene)-(het), -(hetaryl), or —(C₁₋₄-alkylene)-(hetaryl), wherein R³ is optionally substituted by one or more groups independently selected from H, OH, -oxo, —COOH, C₁₋₃ alkyl, C₁₋₃ haloalkyl, C₁₋₃ alkyl OH, —CO—NH₂, —(C₁₋₄-alkylene)-CO—NH₂, —CO—NH(C₁₋₃-alkyl), —(C₁₋₄-alkylene)—CO—NH(C₁₋₃-alkyl), —CO—N(C₁₋₃-alkyl)₂, alkylene)-CO—N(C₁₋₃-alkyl)₂, —NH—(CO)ₘ—NH₂, —NH—(C₁₋₄-alkylene)-(CO)ₘ—NH₂, —NH—(CO)ₘ—NH(C₁₋₃-alkyl), —NH—(C₁₋₄-alkylene)-(CO)ₘ—NH(C₁₋₃-alkyl), —NH—(CO)ₘ—N(C₁₋₃-alkyl)₂, and —NH—(C₁₋₄-alkylene)-(CO)ₘ—N(C₁₋₃-alkyl)₂, or a pharmaceutically acceptable salts thereof.

11. The compounds of formula 1 according to claim 1, wherein R¹ is
a five- to ten-membered, mono- or bicyclic heteroaryl with 1-3 heteroatoms independently selected from N, O, and S, wherein at least of one of the 1-3 heteroatoms is an N atom, and
a three- to ten-membered, mono- or bicyclic, saturated or partially saturated heterocyclic group with 1-3 heteroatoms independently selected from N, O, and S, wherein at least one of the 1-3 heteroatoms is an N atom,
wherein the above-mentioned heteroaryls and heterocycles are each linked via this at least one N atom to the structure according to formula 1, or
wherein R¹ is
a 5- to 11-membered spiro group which contains 1, 2, or 3 heteroatoms independently selected from N, Q and S, wherein at least one of the 1-3 heteroatoms of this spiro group is an N atom and wherein the spiro group is linked via this N atom to the structure according to formula 1,
or a pharmaceutically acceptable salts thereof.

12. The compounds of formula 1 according to claim 1, wherein R¹ is selected from 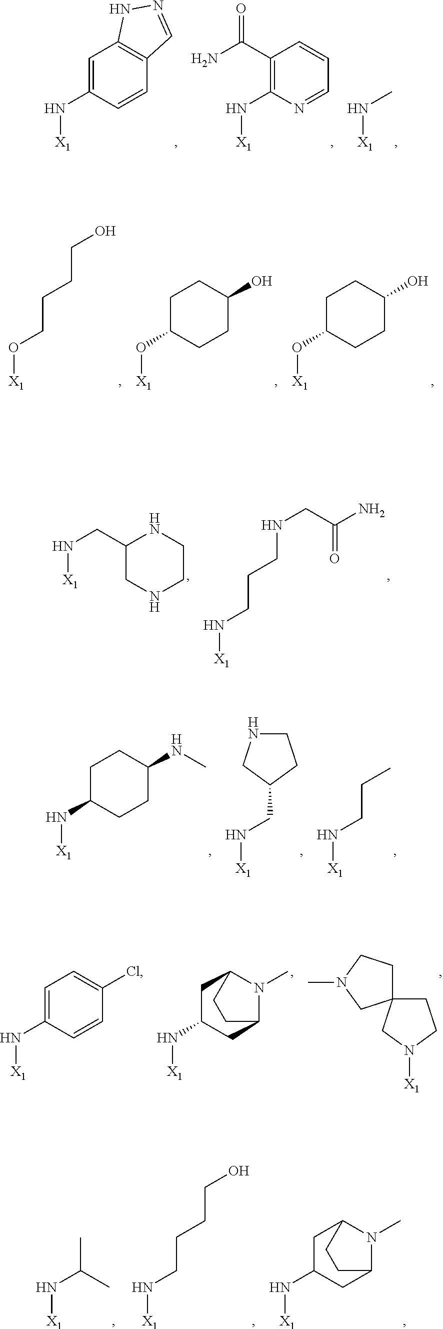 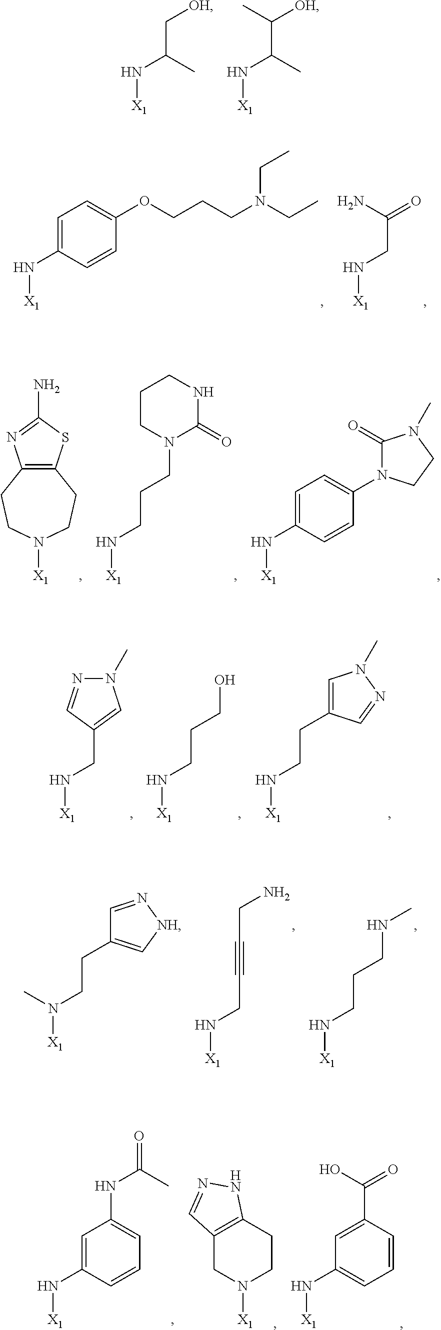 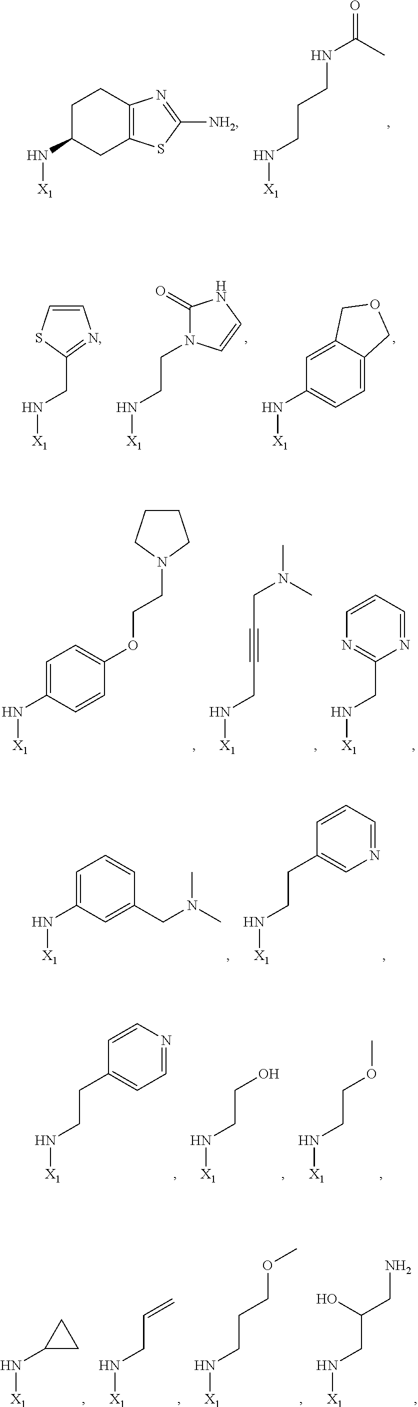 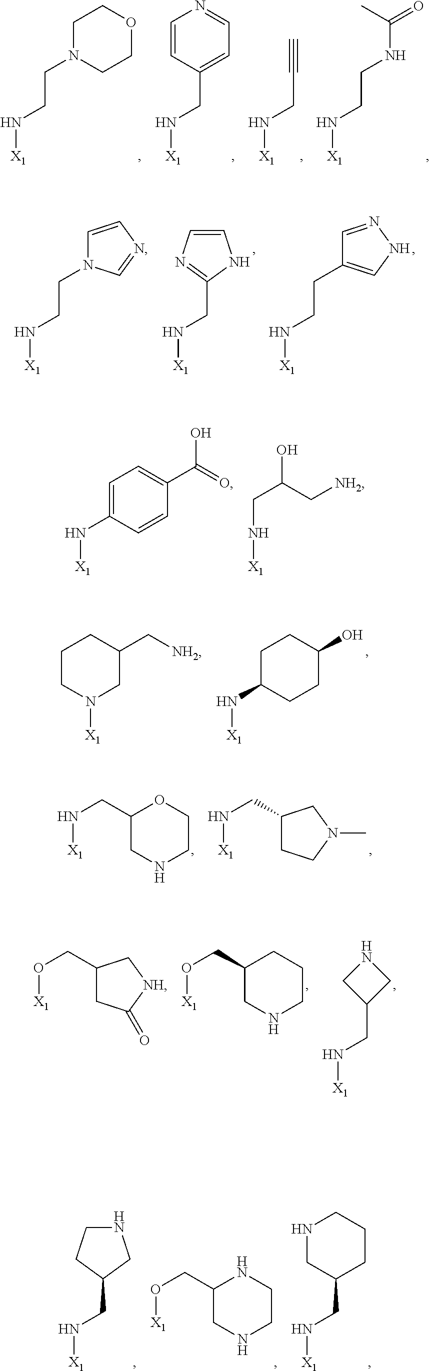 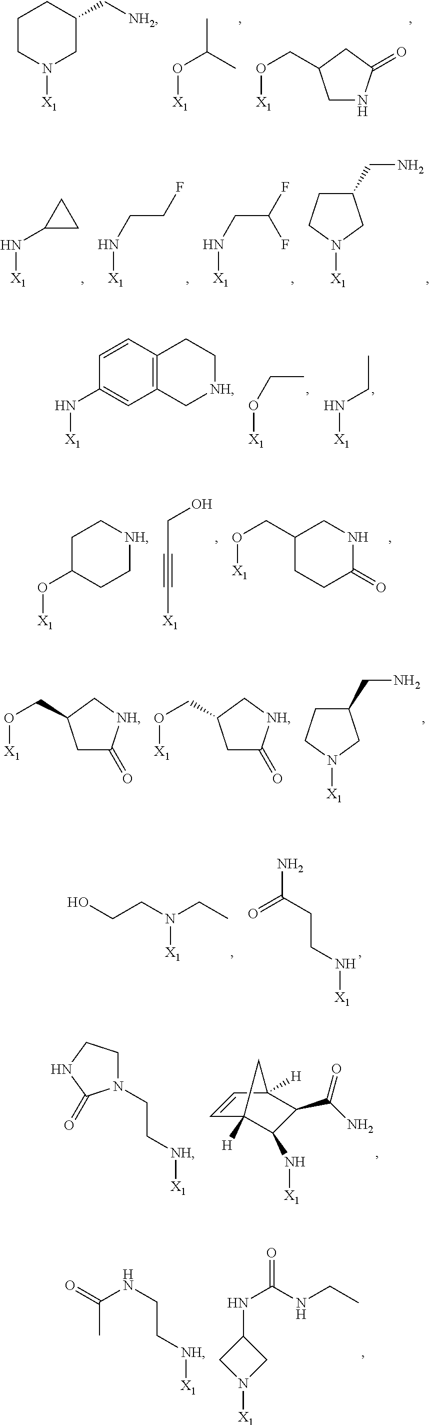 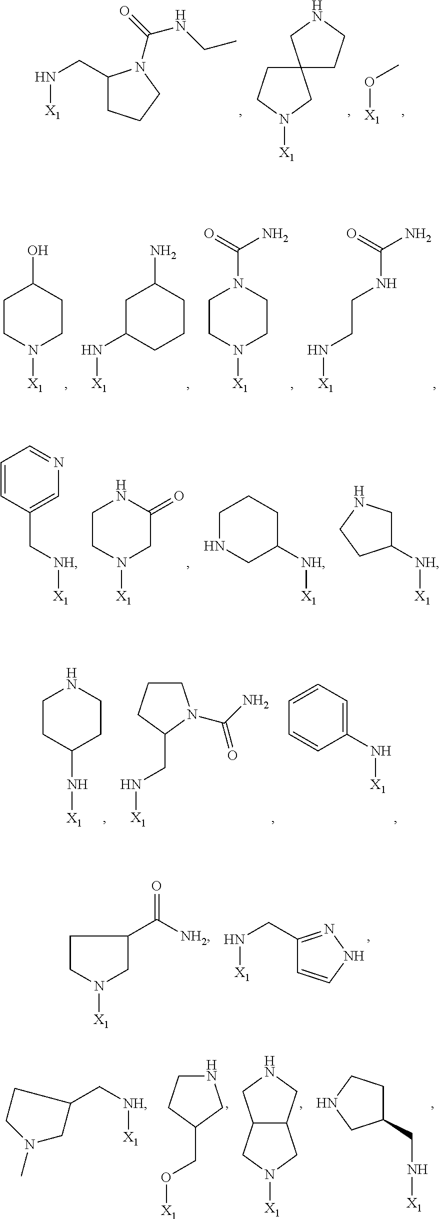 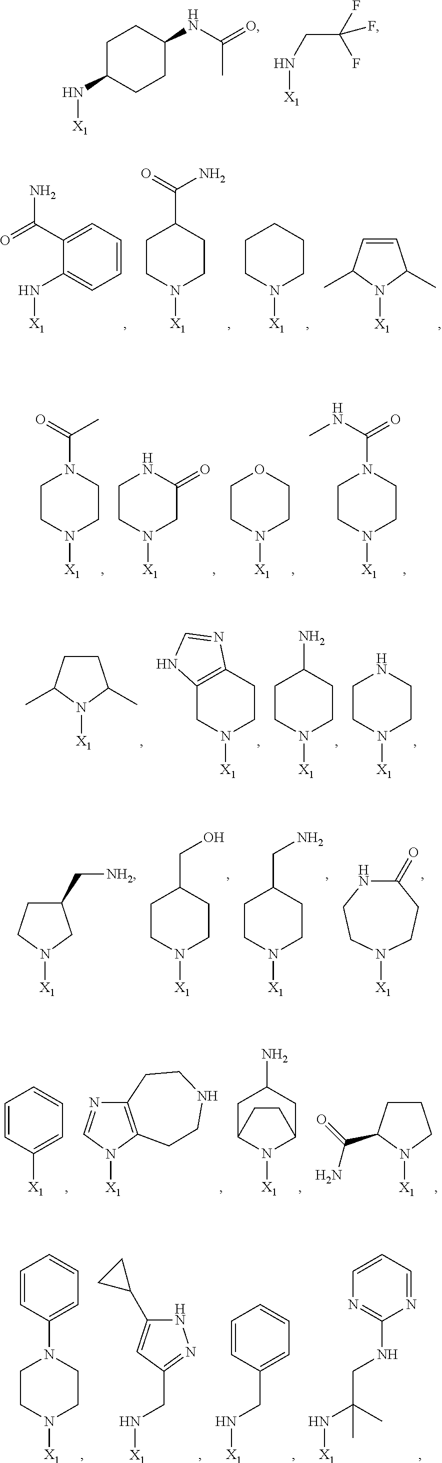 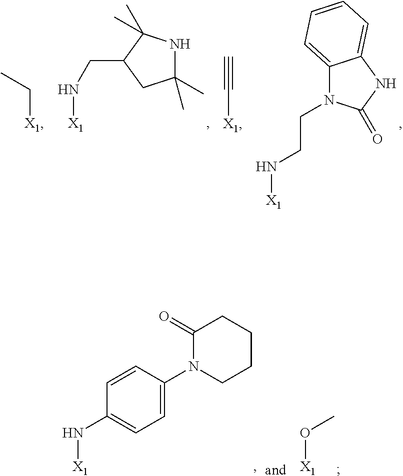
R² is selected from 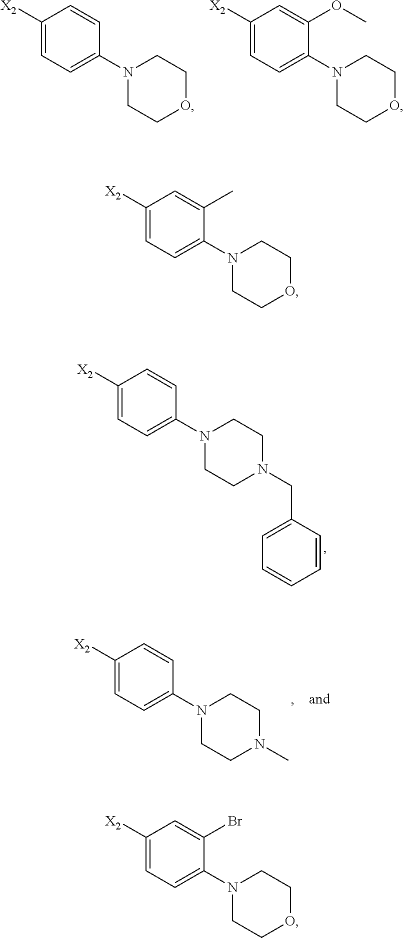
wherein X₁ denotes the point of attachment of R¹ to the structure of formula 1 and X₂ denotes the point of attachment of R² to the structure of formula 1,
or a pharmaceutically acceptable salts thereof.

13. The compounds of formula 1 according to claim 2, wherein R⁶ and R^{6′} are each independently H, methyl, or —OCH₃ or a pharmaceutically acceptable salt thereof.

14. A method for inhibiting the SYK enzyme in a patient in need thereof, comprising administering an effective amount of a compound of claim 1.

15. A method for treating a diseases selected from allergic rhinitis, asthma, COPD, adult respiratory distress syndrome, bronchitis, B-cell lymphoma, dermatitis and contact dermatitis, allergic dermatitis, allergic rhinoconjunctivitis, rheumatoid arthritis, anti-phospholipid syndrome, Berger's disease, Evans's syndrome, ulcerative colitis, allergic antibody-based glomerulonephritis, granulocytopenia, Goodpasture's syndrome, hepatitis, Henoch-Schönlein purpura, hypersensitivity vasculitis, immunohaemolytic anaemia, idiopathic thrombocytopenic purpura, Kawasaki syndrome, allergic conjunctivitis, lupus erythematodes, capsule cell lymphoma, neutropenia, non-familial lateral sclerosis, Crohn's disease, multiple sclerosis, myasthenia gravis, osteoporosis, osteolytic diseases, osteopenia, psoriasis, Sjögren's syndrome, sclerodermy, T-cell lymphoma, urticaria/angiooedema, Wegener's granulomatosis, and coeliac disease in a patient in need thereof, comprising administering an effective amount of a compound according to claim 1.

16. The method according to claim 15, wherein the disease is selected from asthma, COPD, allergic rhinitis, adult respiratory distress syndrome, bronchitis, allergic dermatitis, contact dermatitis, idiopathic thrombocytopenic purpura, rheumatoid arthritis, and allergic rhinoconjunctivitis.

17. The method according to claim 15, wherein the disease is selected from asthma, COPD, allergic rhinitis, allergic dermatitis, and rheumatoid arthritis.

18. A pharmaceutical formulations comprising a compound of formula 1 according to claim 1 and a pharmaceutically acceptable excipient.

19. The pharmaceutical formulation according to claim 1, further comprising an additional active substance selected from betamimetics, corticosteroids, PDE4-inhibitors, EGFR-inhibitors and LTD4-antagonists, CCR3-inhibitors, iNOS-inhibitors, and SYK-inhibitors.

20. A compounds selected from 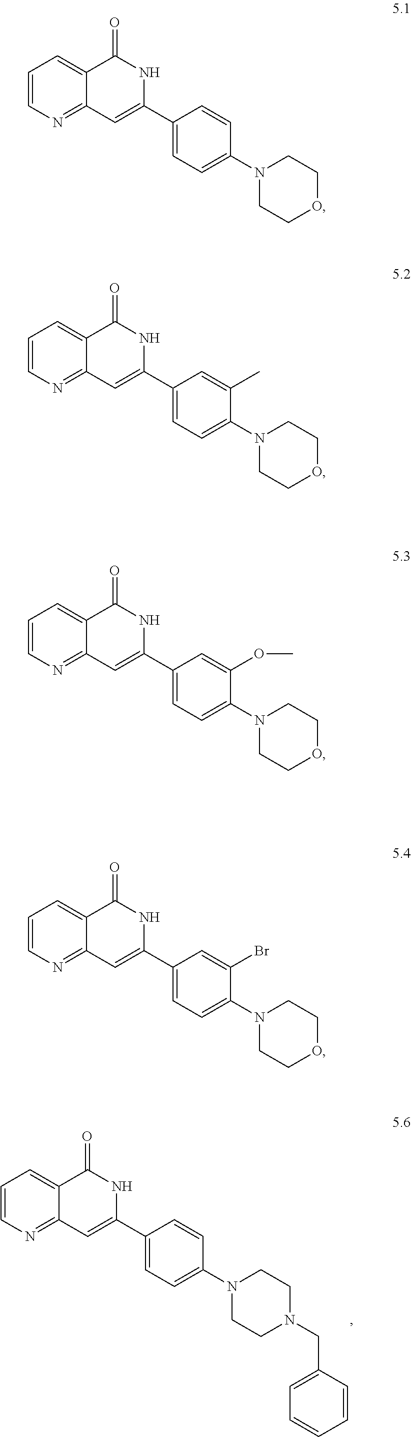 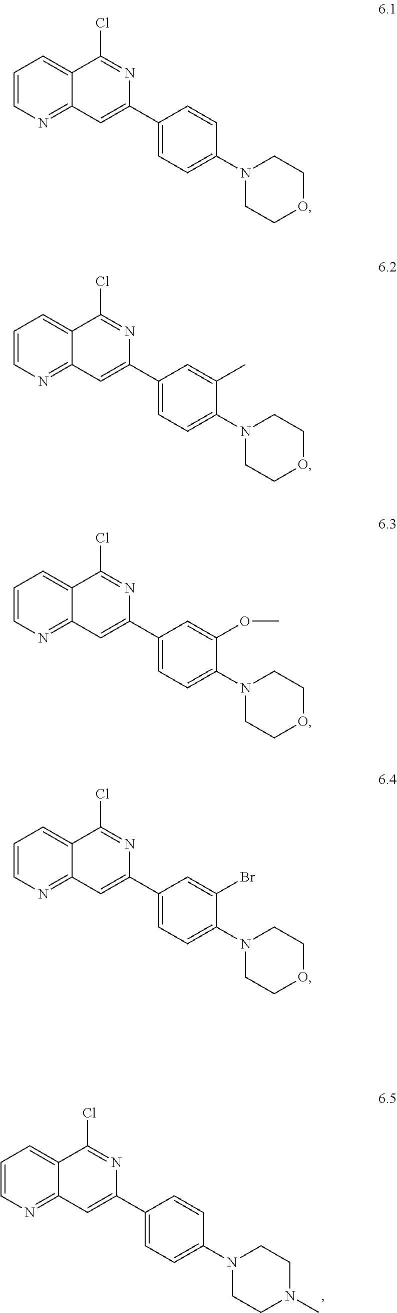 [chemical structure]
